SEQUENCE LISTING (20 sequences)

SEQ ID NO: 1 - DNA, 2433 bp, Homo sapiens
  CDS (1)...(2433): Encodes full-length human plasminogen
atggaacataaggaagtggttcttctacttcttttatttctgaaatcaggtcaaggagagcctctggatgactatgtgaatacccagggggcttcactgttcagtgtcactaagaagcagctgggagcaggaagtatagaagaatgtgcagcaaaatgtgaggaggacgaagaattcacctgcagggcattccaatatcacagtaaagagcaacaatgtgtgataatggctgaaaacaggaagtcctccataatcattaggatgagagatgtagttttatttgaaaagaaagtgtatctctcagagtgcaagactgggaatggaaagaactacagagggacgatgtccaaaacaaaaaatggcatcacctgtcaaaaatggagttccacttctccccacagacctagattctcacctgctacacacccctcagagggactggaggagaactactgcaggaatccagacaacgatccgcaggggccctggtgctatactactgatccagaaaagagatatgactactgcgacattcttgagtgtgaagaggaatgtatgcattgcagtggagaaaactatgacggcaaaatttccaagaccatgtctggactggaatgccaggcctgggactctcagagcccacacgctcatggatacattccttccaaatttccaaacaagaacctgaagaagaattactgtcgtaaccccgatagggagctgcggccttggtgtttcaccaccgaccccaacaagcgctgggaactttgtgacatcccccgctgcacaacacctccaccatcttctggtcccacctaccagtgtctgaagggaacaggtgaaaactatcgcgggaatgtggctgttaccgtgtccgggcacacctgtcagcactggagtgcacagacccctcacacacataacaggacaccagaaaacttcccctgcaaaaatttggatgaaaactactgccgcaatcctgacggaaaaagggccccatggtgccatacaaccaacagccaagtgcggtgggagtactgtaagataccgtcctgtgactcctccccagtatccacggaacaattggctcccacagcaccacctgagctaacccctgtggtccaggactgctaccatggtgatggacagagctaccgaggcacatcctccaccaccaccacaggaaagaagtgtcagtcttggtcatctatgacaccacaccggcaccagaagaccccagaaaactacccaaatgctggcctgacaatgaactactgcaggaatccagatgccgataaaggcccctggtgttttaccacagaccccagcgtcaggtgggagtactgcaacctgaaaaaatgctcaggaacagaagcgagtgttgtagcacctccgcctgttgtcctgcttccagatgtagagactccttccgaagaagactgtatgtttgggaatgggaaaggataccgaggcaagagggcgaccactgttactgggacgccatgccaggactgggctgcccaggagccccatagacacagcattttcactccagagacaaatccacgggcgggtctggaaaaaaattactgccgtaaccctgatggtgatgtaggtggtccctggtgctacacgacaaatccaagaaaactttacgactactgtgatgtccctcagtgtgcggccccttcatttgattgtgggaagcctcaagtggagccgaagaaatgtcctggaagggttgtaggggggtgtgtggcccacccacattcctggccctggcaagtcagtcttagaacaaggtttggaatgcacttctgtggaggcaccttgatatccccagagtgggtgttgactgctgcccactgcttggagaagtccccaaggccttcatcctacaaggtcatcctgggtgcacaccaagaagtgaatctcgaaccgcatgttcaggaaatagaagtgtctaggctgttcttggagcccacacgaaaagatattgccttgctaaagctaagcagtcctgccgtcatcactgacaaagtaatcccagcttgtctgccatccccaaattatgtggtcgctgaccggaccgaatgtttcgtcactggctggggagaaacccaaggtacttttggagctggccttctcaaggaagcccagctccctgtgattgagaataaagtgtgcaatcgctatgagtttctgaatggaagagtccaatccaccgaactctgtgctgggcatttggccggaggcactgacagttgccagggtgacagtggaggtcctctggtttgcttcgagaaggacaaatacattttacaaggagtcacttcttggggtcttggctgtgcacgccccaataagcctggtgtctatgttcgtgtttcaaggtttgttacttggattgagggagtgatgagaaataattaa

SEQ ID NO: 2 - protein, 810 aa, Homo sapiens [sequence omitted]

SEQ ID NO: 3 - DNA, 2376 bp, Homo sapiens
  CDS (1)...(2376): Encodes mature human plasminogen
gagcctctggatgactatgtgaatacccagggggcttcactgttcagtgtcactaagaagcagctgggagcaggaagtatagaagaatgtgcagcaaaatgtgaggaggacgaagaattcacctgcagggcattccaatatcacagtaaagagcaacaatgtgtgataatggctgaaaacaggaagtcctccataatcattaggatgagagatgtagttttatttgaaaagaaagtgtatctctcagagtgcaagactgggaatggaaagaactacagagggacgatgtccaaaacaaaaaatggcatcacctgtcaaaaatggagttccacttctccccacagacctagattctcacctgctacacacccctcagagggactggaggagaactactgcaggaatccagacaacgatccgcaggggccctggtgctatactactgatccagaaaagagatatgactactgcgacattcttgagtgtgaagaggaatgtatgcattgcagtggagaaaactatgacggcaaaatttccaagaccatgtctggactggaatgccaggcctgggactctcagagcccacacgctcatggatacattccttccaaatttccaaacaagaacctgaagaagaattactgtcgtaaccccgatagggagctgcggccttggtgtttcaccaccgaccccaacaagcgctgggaactttgtgacatcccccgctgcacaacacctccaccatcttctggtcccacctaccagtgtctgaagggaacaggtgaaaactatcgcgggaatgtggctgttaccgtgtccgggcacacctgtcagcactggagtgcacagacccctcacacacataacaggacaccagaaaacttcccctgcaaaaatttggatgaaaactactgccgcaatcctgacggaaaaagggccccatggtgccatacaaccaacagccaagtgcggtgggagtactgtaagataccgtcctgtgactcctccccagtatccacggaacaattggctcccacagcaccacctgagctaacccctgtggtccaggactgctaccatggtgatggacagagctaccgaggcacatcctccaccaccaccacaggaaagaagtgtcagtcttggtcatctatgacaccacaccggcaccagaagaccccagaaaactacccaaatgctggcctgacaatgaactactgcaggaatccagatgccgataaaggcccctggtgttttaccacagaccccagcgtcaggtgggagtactgcaacctgaaaaaatgctcaggaacagaagcgagtgttgtagcacctccgcctgttgtcctgcttccagatgtagagactccttccgaagaagactgtatgtttgggaatgggaaaggataccgaggcaagagggcgaccactgttactgggacgccatgccaggactgggctgcccaggagccccatagacacagcattttcactccagagacaaatccacgggcgggtctggaaaaaaattactgccgtaaccctgatggtgatgtaggtggtccctggtgctacacgacaaatccaagaaaactttacgactactgtgatgtccctcagtgtgcggccccttcatttgattgtgggaagcctcaagtggagccgaagaaatgtcctggaagggttgtaggggggtgtgtggcccacccacattcctggccctggcaagtcagtcttagaacaaggtttggaatgcacttctgtggaggcaccttgatatccccagagtgggtgttgactgctgcccactgcttggagaagtccccaaggccttcatcctacaaggtcatcctgggtgcacaccaagaagtgaatctcgaaccgcatgttcaggaaatagaagtgtctaggctgttcttggagcccacacgaaaagatattgccttgctaaagctaagcagtcctgccgtcatcactgacaaagtaatcccagcttgtctgccatccccaaattatgtggtcgctgaccggaccgaatgtttcgtcactggctggggagaaacccaaggtacttttggagctggccttctcaaggaagcccagctccctgtgattgagaataaagtgtgcaatcgctatgagtttctgaatggaagagtccaatccaccgaactctgtgctgggcatttggccggaggcactgacagttgccagggtgacagtggaggtcctctggtttgcttcgagaaggacaaatacattttacaaggagtcacttcttggggtcttggctgtgcacgccccaataagcctggtgtctatgttcgtgtttcaaggtttgttacttggattgagggagtgatgagaaataattaa

SEQ ID NO: 4 - protein, 791 aa, Homo sapiens
  Sequence of mature human plasminogen
EPLDDYVNTQGASLFSVTKKQLGAGSIEECAAKCEEDEEFTCRAFQYHSKEQQCVIMAENRKSSIIIRMRDVVLFEKKVYLSECKTGNGKNYRGTMSKTKNGITCQKWSSTSPHRPRFSPATHPSEGLEENYCRNPDNDPQGPWCYTTDPEKRYDYCDILECEEECMHCSGENYDGKISKTMSGLECQAWDSQSPHAHGYIPSKFPNKNLKKNYCRNPDRELRPWCFTTDPNKRWELCDIPRCTTPPPSSGPTYQCLKGTGENYRGNVAVTVSGHTCQHWSAQTPHTHNRTPENFPCKNLDENYCRNPDGKRAPWCHTTNSQVRWEYCKIPSCDSSPVSTEQLAPTAPPELTPVVQDCYHGDGQSYRGTSSTTTTGKKCQSWSSMTPHRHQKTPENYPNAGLTMNYCRNPDADKGPWCFTTDPSVRWEYCNLKKCSGTEASVVAPPPVVLLPDVETPSEEDCMFGNGKGYRGKRATTVTGTPCQDWAAQEPHRHSIFTPETNPRAGLEKNYCRNPDGDVGGPWCYTTNPRKLYDYCDVPQCAAPSFDCGKPQVEPKKCPGRVVGGCVAHPHSWPWQVSLRTRFGMHFCGGTLISPEWVLTAAHCLEKSPRPSSYKVILGAHQEVNLEPHVQEIEVSRLFLEPTRKDIALLKLSSPAVITDKVIPACLPSPNYVVADRTECFVTGWGETQGTFGAGLLKEAQLPVIENKVCNRYEFLNGRVQSTELCAGHLAGGTDSCQGDSGGPLVCFEKDKYILQGVTSWGLGCARPNKPGVYVRVSRFVTWIEGVMRNN

SEQ ID NO: 5 - DNA, 2376 bp, Artificial Sequence
  Duckweed codon-optimized sequence encoding N289D variant of mature human plasminogen
gagcccctggacgactacgtgaacacgcagggcgcctccctgttcagcgtcaccaagaagcagctcggcgccggcagcatcgaggagtgcgcggccaagtgcgaggaggacgaggagttcacgtgccgcgccttccagtaccacagcaaggagcagcagtgcgtgatcatggccgagaaccgcaagtcctccatcatcatccgcatgcgcgacgtggtcctcttcgagaagaaggtgtatctgtccgagtgcaagacgggcaacgggaagaactaccgcgggacgatgtccaagaccaagaacggcatcacctgccaaaagtggtcttccacgagcccgcaccgcccgcgcttctcccctgccacgcacccctccgagggactcgaagagaactactgccgcaaccccgacaacgacccgcagggcccgtggtgctacacgaccgacccggagaaacggtacgactactgcgacattctcgagtgcgaggaggagtgcatgcactgcagcggcgagaattacgacggcaagatctccaagactatgtccggcctggagtgccaggcctgggactcccagagcccgcacgcccatgggtacatcccgtccaagttccccaacaagaacctgaagaagaactactgcaggaaccccgaccgcgagcttcgcccgtggtgcttcacgaccgacccgaacaagcgctgggagttgtgcgacatcccgcgttgcaccacgcctccgccctccagcgggccgacctatcagtgcctgaaaggcacgggcgagaactaccgcggaaacgtggcggtcaccgtgtccgggcacacctgccaacactggagcgcgcagaccccgcacacccacgaccggacgccggagaacttcccctgcaagaacctggacgagaactactgccgcaacccggacgggaagcgtgccccgtggtgccacaccaccaacagccaggttcgctgggagtactgcaagatcccgtcctgcgattcctctccggtctccactgagcagctggcgcccaccgcgccgcccgaactcacgccggtggtccaggactgctatcacggcgacgggcagagctaccgcggcacctcttccaccactaccacgggcaagaagtgccagtcctggtccagcatgacccctcaccggcaccagaagaccccggagaactacccgaacgccggcctgaccatgaactactgcaggaacccggacgccgacaaaggcccctggtgcttcaccaccgatcccagcgtccgctgggagtactgcaacctgaagaaatgctccggcaccgaggcgagcgtcgtcgcgccgccacccgtggtcctgcttccggacgtcgagaccccgtccgaggaggactgcatgttcgggaacgggaagggctaccgcggcaagcgcgcgaccactgtcaccgggaccccgtgccaggactgggccgctcaggagccccacaggcacagcatcttcaccccggagaccaacccgcgcgcgggcctggagaagaactactgccgcaacccggacggcgacgttggaggcccctggtgctacactaccaacccgcgtaagctctacgactactgcgacgtcccgcagtgcgcggctccgtccttcgactgcgggaagccacaggtggaaccgaagaagtgccctggccgcgtggtcggagggtgcgtggcccacccgcactcctggccctggcaagtcagcctgcgcacccgcttcggcatgcacttctgcggcggcaccctcatctccccggagtgggttctgaccgccgctcactgcctcgagaagtccccgaggccctcctcctacaaggtcatcctgggcgcccaccaggaggtgaacctcgagccgcacgttcaggagatcgaggtgtcccgcttgttcctggagcccacgcgcaaggatatcgccctgctcaagctctctagcccggccgtcatcaccgacaaggttatcccggcctgccttccctccccgaactacgtggtcgctgaccgcaccgagtgcttcgttaccggctggggcgagacccagggaacgttcggcgcgggcctcctcaaggaggcccagctcccggtgattgagaacaaggtgtgcaaccgttacgagttcctgaacgggcgcgtccagtccaccgaactctgcgccgggcacttggccggcggcaccgacagctgccagggcgacagcggcgggccgctggtgtgcttcgagaaggacaagtacatcctccaaggcgtcacgtcctggggcctcggctgcgcacgccctaacaagccgggcgtctatgtgcgcgtgtcccgcttcgtgacctggatcgagggcgtgatgcgcaacaactaa

SEQ ID NO: 6 - protein, 791 aa, Artificial Sequence
  Sequence of N289D variant of mature human plasminogen
EPLDDYVNTQGASLFSVTKKQLGAGSIEECAAKCEEDEEFTCRAFQYHSKEQQCVIMAENRKSSIIIRMRDVVLFEKKVYLSECKTGNGKNYRGTMSKTKNGITCQKWSSTSPHRPRFSPATHPSEGLEENYCRNPDNDPQGPWCYTTDPEKRYDYCDILECEEECMHCSGENYDGKISKTMSGLECQAWDSQSPHAHGYIPSKFPNKNLKKNYCRNPDRELRPWCFTTDPNKRWELCDIPRCTTPPPSSGPTYQCLKGTGENYRGNVAVTVSGHTCQHWSAQTPHTHDRTPENFPCKNLDENYCRNPDGKRAPWCHTTNSQVRWEYCKIPSCDSSPVSTEQLAPTAPPELTPVVQDCYHGDGQSYRGTSSTTTTGKKCQSWSSMTPHRHQKTPENYPNAGLTMNYCRNPDADKGPWCFTTDPSVRWEYCNLKKCSGTEASVVAPPPVVLLPDVETPSEEDCMFGNGKGYRGKRATTVTGTPCQDWAAQEPHRHSIFTPETNPRAGLEKNYCRNPDGDVGGPWCYTTNPRKLYDYCDVPQCAAPSFDCGKPQVEPKKCPGRVVGGCVAHPHSWPWQVSLRTRFGMHFCGGTLISPEWVLTAAHCLEKSPRPSSYKVILGAHQEVNLEPHVQEIEVSRLFLEPTRKDIALLKLSSPAVITDKVIPACLPSPNYVVADRTECFVTGWGETQGTFGAGLLKEAQLPVIENKVCNRYEFLNGRVQSTELCAGHLAGGTDSCQGDSGGPLVCFEKDKYILQGVTSWGLGCARPNKPGVYVRVSRFVTWIEGVMRNN

SEQ ID NO: 7 - DNA, 93 bp, Artificial Sequence
  Duckweed codon-optimized sequence encoding rice alpha-amylase signal peptide
atgcaggtcctgaacacgatggtcaacaagcacttcctctccctgtccgtcctcatcgtcctcctcgggctgagcagcaacctcaccgccggc

SEQ ID NO: 8 - protein, 31 aa, Oryza sativa
  Sequence of rice alpha-amylase signal peptide
MQVLNTMVNKHFLSLSVLIVLLGLSSNLTAG

SEQ ID NO: 9 - DNA, 554 bp, Zea mays
  Maize ADH1 intron
gatcaagtgcaaaggtccgccttgtttctcctctgtctcttgatctgactaatcttggtttatgattcgttgagtaattttggggaaagcttcgtccacagtttttttttcgatgaacagtgccgcagtggcgctgatcttgtatgctatcctgcaatcgtggtgaacttatgtcttttatatccttcactaccatgaaaagactagtaatctttctcgatgtaacatcgtccagcactgctattaccgtgtggtccatccgacagtctggctgaacacatcatacgatattgagcaaagatctatcttccctgttctttaatgaaagacgtcattttcatcagtatgatctaagaatgttgcaacttgcaaggaggcgtttctttctttgaatttaactaactcgttgagtggccctgtttctcggacgtaaggcctttgctgctccacacatgtccattcgaattttaccgtgtttagcaagggcgaaaagtttgcatcttgatgatttagcttgactatgcgattgctttcctggacccgtgcagctgcgg

SEQ ID NO: 10 - DNA, 63 bp, Lemna gibba
  5′translation leader from the ribulose-bis- phosphate carboxylase small subunit 5B gene
gaaactcccgaggtgagcaaggatccggagtcgagcgcgaagaagagaaagagggaaagcgcg

SEQ ID NO: 11 - DNA, 2469 bp, Artificial Sequence
  Duckweed codon-optimized sequence encoding rice alpha-amylase signal peptide/N289D variant of mature human plasminogen
atgcaggtcctgaacacgatggtcaacaagcacttcctctccctgtccgtcctcatcgtcctcctcgggctgagcagcaacctcaccgccggcgagcccctggacgactacgtgaacacgcagggcgcctccctgttcagcgtcaccaagaagcagctcggcgccggcagcatcgaggagtgcgcggccaagtgcgaggaggacgaggagttcacgtgccgcgccttccagtaccacagcaaggagcagcagtgcgtgatcatggccgagaaccgcaagtcctccatcatcatccgcatgcgcgacgtggtcctcttcgagaagaaggtgtatctgtccgagtgcaagacgggcaacgggaagaactaccgcgggacgatgtccaagaccaagaacggcatcacctgccaaaagtggtcttccacgagcccgcaccgcccgcgcttctcccctgccacgcacccctccgagggactcgaagagaactactgccgcaaccccgacaacgacccgcagggcccgtggtgctacacgaccgacccggagaaacggtacgactactgcgacattctcgagtgcgaggaggagtgcatgcactgcagcggcgagaattacgacggcaagatctccaagactatgtccggcctggagtgccaggcctgggactcccagagcccgcacgcccatgggtacatcccgtccaagttccccaacaagaacctgaagaagaactactgcaggaaccccgaccgcgagcttcgcccgtggtgcttcacgaccgacccgaacaagcgctgggagttgtgcgacatcccgcgttgcaccacgcctccgccctccagcgggccgacctatcagtgcctgaaaggcacgggcgagaactaccgcggaaacgtggcggtcaccgtgtccgggcacacctgccaacactggagcgcgcagaccccgcacacccacgaccggacgccggagaacttcccctgcaagaacctggacgagaactactgccgcaacccggacgggaagcgtgccccgtggtgccacaccaccaacagccaggttcgctgggagtactgcaagatcccgtcctgcgattcctctccggtctccactgagcagctggcgcccaccgcgccgcccgaactcacgccggtggtccaggactgctatcacggcgacgggcagagctaccgcggcacctcttccaccactaccacgggcaagaagtgccagtcctggtccagcatgacccctcaccggcaccagaagaccccggagaactacccgaacgccggcctgaccatgaactactgcaggaacccggacgccgacaaaggcccctggtgcttcaccaccgatcccagcgtccgctgggagtactgcaacctgaagaaatgctccggcaccgaggcgagcgtcgtcgcgccgccacccgtggtcctgcttccggacgtcgagaccccgtccgaggaggactgcatgttcgggaacgggaagggctaccgcggcaagcgcgcgaccactgtcaccgggaccccgtgccaggactgggccgctcaggagccccacaggcacagcatcttcaccccggagaccaacccgcgcgcgggcctggagaagaactactgccgcaacccggacggcgacgttggaggcccctggtgctacactaccaacccgcgtaagctctacgactactgcgacgtcccgcagtgcgcggctccgtccttcgactgcgggaagccacaggtggaaccgaagaagtgccctggccgcgtggtcggagggtgcgtggcccacccgcactcctggccctggcaagtcagcctgcgcacccgcttcggcatgcacttctgcggcggcaccctcatctccccggagtgggttctgaccgccgctcactgcctcgagaagtccccgaggccctcctcctacaaggtcatcctgggcgcccaccaggaggtgaacctcgagccgcacgttcaggagatcgaggtgtcccgcttgttcctggagcccacgcgcaaggatatcgccctgctcaagctctctagcccggccgtcatcaccgacaaggttatcccggcctgccttccctccccgaactacgtggtcgctgaccgcaccgagtgcttcgttaccggctggggcgagacccagggaacgttcggcgcgggcctcctcaaggaggcccagctcccggtgattgagaacaaggtgtgcaaccgttacgagttcctgaacgggcgcgtccagtccaccgaactctgcgccgggcacttggccggcggcaccgacagctgccagggcgacagcggcgggccgctggtgtgcttcgagaaggacaagtacatcctccaaggcgtcacgtcctggggcctcggctgcgcacgccctaacaagccgggcgtctatgtgcgcgtgtcccgcttcgtgacctggatcgagggcgtgatgcgcaacaactaa

SEQ ID NO: 12 - protein, 822 aa, Artificial Sequence
  Sequence of rice alpha-amylase signal peptide/N289D variant of mature human plasminogen
MQVLNTMVNKHFLSLSVLIVLLGLSSNLTAGEPLDDYVNTQGASLFSVTKKQLGAGSIEECAAKCEEDEEFTCRAFQYHSKEQQCVIMAENRKSSIIIRMRDVVLFEKKVYLSECKTGNGKNYRGTMSKTKNGITCQKWSSTSPHRPRFSPATHPSEGLEENYCRNPDNDPQGPWCYTTDPEKRYDYCDILECEEECMHCSGENYDGKISKTMSGLECQAWDSQSPHAHGYIPSKFPNKNLKKNYCRNPDRELRPWCFTTDPNKRWELCDIPRCTTPPPSSGPTYQCLKGTGENYRGNVAVTVSGHTCQHWSAQTPHTHDRTPENFPCKNLDENYCRNPDGKRAPWCHTTNSQVRWEYCKIPSCDSSPVSTEQLAPTAPPELTPVVQDCYHGDGQSYRGTSSTTTTGKKCQSWSSMTPHRHQKTPENYPNAGLTMNYCRNPDADKGPWCFTTDPSVRWEYCNLKKCSGTEASVVAPPPVVLLPDVETPSEEDCMFGNGKGYRGKRATTVTGTPCQDWAAQEPHRHSIFTPETNPRAGLEKNYCRNPDGDVGGPWCYTTNPRKLYDYCDVPQCAAPSFDCGKPQVEPKKCPGRVVGGCVAHPHSWPWQVSLRTRFGMHFCGGTLISPEWVLTAAHCLEKSPRPSSYKVILGAHQEVNLEPHVQEIEVSRLFLEPTRKDIALLKLSSPAVITDKVIPACLPSPNYVVADRTECFVTGWGETQGTFGAGLLKEAQLPVIENKVCNRYEFLNGRVQSTELCAGHLAGGTDSCQGDSGGPLVCFEKDKYILQGVTSWGLGCARPNKPGVYVRVSRFVTWIEGVMRNN

SEQ ID NO: 13 - DNA, 2433 bp, Homo sapiens
  CDS (1)...(2433): Encodes V701I variant of full-length human plasminogen of SEQ ID NO2
atggaacataaggaagtggttcttctacttcttttatttctgaaatcaggtcaaggagagcctctggatgactatgtgaatacccagggggcttcactgttcagtgtcactaagaagcagctgggagcaggaagtatagaagaatgtgcagcaaaatgtgaggaggacgaagaattcacctgcagggcattccaatatcacagtaaagagcaacaatgtgtgataatggctgaaaacaggaagtcctccataatcattaggatgagagatgtagttttatttgaaaagaaagtgtatctctcagagtgcaagactgggaatggaaagaactacagagggacgatgtccaaaacaaaaaatggcatcacctgtcaaaaatggagttccacttctccccacagacctagattctcacctgctacacacccctcagagggactggaggagaactactgcaggaatccagacaacgatccgcaggggccctggtgctatactactgatccagaaaagagatatgactactgcgacattcttgagtgtgaagaggaatgtatgcattgcagtggagaaaactatgacggcaaaatttccaagaccatgtctggactggaatgccaggcctgggactctcagagcccacacgctcatggatacattccttccaaatttccaaacaagaacctgaagaagaattactgtcgtaaccccgatagggagctgcggccttggtgtttcaccaccgaccccaacaagcgctgggaactttgtgacatcccccgctgcacaacacctccaccatcttctggtcccacctaccagtgtctgaagggaacaggtgaaaactatcgcgggaatgtggctgttaccgtgtccgggcacacctgtcagcactggagtgcacagacccctcacacacataacaggacaccagaaaacttcccctgcaaaaatttggatgaaaactactgccgcaatcctgacggaaaaagggccccatggtgccatacaaccaacagccaagtgcggtgggagtactgtaagataccgtcctgtgactcctccccagtatccacggaacaattggctcccacagcaccacctgagctaacccctgtggtccaggactgctaccatggtgatggacagagctaccgaggcacatcctccaccaccaccacaggaaagaagtgtcagtcttggtcatctatgacaccacaccggcaccagaagaccccagaaaactacccaaatgctggcctgacaatgaactactgcaggaatccagatgccgataaaggcccctggtgttttaccacagaccccagcgtcaggtgggagtactgcaacctgaaaaaatgctcaggaacagaagcgagtgttgtagcacctccgcctgttgtcctgcttccagatgtagagactccttccgaagaagactgtatgtttgggaatgggaaaggataccgaggcaagagggcgaccactgttactgggacgccatgccaggactgggctgcccaggagccccatagacacagcattttcactccagagacaaatccacgggcgggtctggaaaaaaattactgccgtaaccctgatggtgatgtaggtggtccctggtgctacacgacaaatccaagaaaactttacgactactgtgatgtccctcagtgtgcggccccttcatttgattgtgggaagcctcaagtggagccgaagaaatgtcctggaagggttgtaggggggtgtgtggcccacccacattcctggccctggcaagtcagtcttagaacaaggtttggaatgcacttctgtggaggcaccttgatatccccagagtgggtgttgactgctgcccactgcttggagaagtccccaaggccttcatcctacaaggtcatcctgggtgcacaccaagaagtgaatctcgaaccgcatgttcaggaaatagaagtgtctaggctgttcttggagcccacacgaaaagatattgccttgctaaagctaagcagtcctgccgtcatcactgacaaagtaatcccagcttgtctgccatccccaaattatgtggtcgctgaccggaccgaatgtttcatcactggctggggagaaacccaaggtacttttggagctggccttctcaaggaagcccagctccctgtgattgagaataaagtgtgcaatcgctatgagtttctgaatggaagagtccaatccaccgaactctgtgctgggcatttggccggaggcactgacagttgccagggtgacagtggaggtcctctggtttgcttcgagaaggacaaatacattttacaaggagtcacttcttggggtcttggctgtgcacgccccaataagcctggtgtctatgttcgtgtttcaaggtttgttacttggattgagggagtgatgagaaataattaa

SEQ ID NO: 14 - protein, 810 aa, Homo sapiens
  VARIANT (1)...(810): Sequence of V701I variant of full-length human plasminogen of SEQ ID NO2
MEHKEVVLLLLLFLKSGQGEPLDDYVNTQGASLFSVTKKQLGAGSIEECAAKCEEDEEFTCRAFQYHSKEQQCVIMAENRKSSIIIRMRDVVLFEKKVYLSECKTGNGKNYRGTMSKTKNGITCQKWSSTSPHRPRFSPATHPSEGLEENYCRNPDNDPQGPWCYTTDPEKRYDYCDILECEEECMHCSGENYDGKISKTMSGLECQAWDSQSPHAHGYIPSKFPNKNLKKNYCRNPDRELRPWCFTTDPNKRWELCDIPRCTTPPPSSGPTYQCLKGTGENYRGNVAVTVSGHTCQHWSAQTPHTHNRTPENFPCKNLDENYCRNPDGKRAPWCHTTNSQVRWEYCKIPSCDSSPVSTEQLAPTAPPELTPVVQDCYHGDGQSYRGTSSTTTTGKKCQSWSSMTPHRHQKTPENYPNAGLTMNYCRNPDADKGPWCFTTDPSVRWEYCNLKKCSGTEASVVAPPPVVLLPDVETPSEEDCMFGNGKGYRGKRATTVTGTPCQDWAAQEPHRHSIFTPETNPRAGLEKNYCRNPDGDVGGPWCYTTNPRKLYDYCDVPQCAAPSFDCGKPQVEPKKCPGRVVGGCVAHPHSWPWQVSLRTRFGMHFCGGTLISPEWVLTAAHCLEKSPRPSSYKVILGAHQEVNLEPHVQEIEVSRLFLEPTRKDIALLKLSSPAVITDKVIPACLPSPNYVVADRTECFITGWGETQGTFGAGLLKEAQLPVIENKVCNRYEFLNGRVQSTELCAGHLAGGTDSCQGDSGGPLVCFEKDKYILQGVTSWGLGCARPNKPGVYVRVSRFVTWIEGVMRNN

SEQ ID NO: 15 - DNA, 2376 bp, Homo sapiens
  CDS (1)...(2376): Encodes V682I variant of mature human plasminogen of SEQ ID NO4
gagcctctggatgactatgtgaatacccagggggcttcactgttcagtgtcactaagaagcagctgggagcaggaagtatagaagaatgtgcagcaaaatgtgaggaggacgaagaattcacctgcagggcattccaatatcacagtaaagagcaacaatgtgtgataatggctgaaaacaggaagtcctccataatcattaggatgagagatgtagttttatttgaaaagaaagtgtatctctcagagtgcaagactgggaatggaaagaactacagagggacgatgtccaaaacaaaaaatggcatcacctgtcaaaaatggagttccacttctccccacagacctagattctcacctgctacacacccctcagagggactggaggagaactactgcaggaatccagacaacgatccgcaggggccctggtgctatactactgatccagaaaagagatatgactactgcgacattcttgagtgtgaagaggaatgtatgcattgcagtggagaaaactatgacggcaaaatttccaagaccatgtctggactggaatgccaggcctgggactctcagagcccacacgctcatggatacattccttccaaatttccaaacaagaacctgaagaagaattactgtcgtaaccccgatagggagctgcggccttggtgtttcaccaccgaccccaacaagcgctgggaactttgtgacatcccccgctgcacaacacctccaccatcttctggtcccacctaccagtgtctgaagggaacaggtgaaaactatcgcgggaatgtggctgttaccgtgtccgggcacacctgtcagcactggagtgcacagacccctcacacacataacaggacaccagaaaacttcccctgcaaaaatttggatgaaaactactgccgcaatcctgacggaaaaagggccccatggtgccatacaaccaacagccaagtgcggtgggagtactgtaagataccgtcctgtgactcctccccagtatccacggaacaattggctcccacagcaccacctgagctaacccctgtggtccaggactgctaccatggtgatggacagagctaccgaggcacatcctccaccaccaccacaggaaagaagtgtcagtcttggtcatctatgacaccacaccggcaccagaagaccccagaaaactacccaaatgctggcctgacaatgaactactgcaggaatccagatgccgataaaggcccctggtgttttaccacagaccccagcgtcaggtgggagtactgcaacctgaaaaaatgctcaggaacagaagcgagtgttgtagcacctccgcctgttgtcctgcttccagatgtagagactccttccgaagaagactgtatgtttgggaatgggaaaggataccgaggcaagagggcgaccactgttactgggacgccatgccaggactgggctgcccaggagccccatagacacagcattttcactccagagacaaatccacgggcgggtctggaaaaaaattactgccgtaaccctgatggtgatgtaggtggtccctggtgctacacgacaaatccaagaaaactttacgactactgtgatgtccctcagtgtgcggccccttcatttgattgtgggaagcctcaagtggagccgaagaaatgtcctggaagggttgtaggggggtgtgtggcccacccacattcctggccctggcaagtcagtcttagaacaaggtttggaatgcacttctgtggaggcaccttgatatccccagagtgggtgttgactgctgcccactgcttggagaagtccccaaggccttcatcctacaaggtcatcctgggtgcacaccaagaagtgaatctcgaaccgcatgttcaggaaatagaagtgtctaggctgttcttggagcccacacgaaaagatattgccttgctaaagctaagcagtcctgccgtcatcactgacaaagtaatcccagcttgtctgccatccccaaattatgtggtcgctgaccggaccgaatgtttcatcactggctggggagaaacccaaggtacttttggagctggccttctcaaggaagcccagctccctgtgattgagaataaagtgtgcaatcgctatgagtttctgaatggaagagtccaatccaccgaactctgtgctgggcatttggccggaggcactgacagttgccagggtgacagtggaggtcctctggtttgcttcgagaaggacaaatacattttacaaggagtcacttcttggggtcttggctgtgcacgccccaataagcctggtgtctatgttcgtgtttcaaggtttgttacttggattgagggagtgatgagaaataattaa

SEQ ID NO: 16 - protein, 791 aa, Homo sapiens
  VARIANT (1)...(791): Sequence of V682I variant of mature human plasminogen of SEQ ID NO4
EPLDDYVNTQGASLFSVTKKQLGAGSIEECAAKCEEDEEFTCRAFQYHSKEQQCVIMAENRKSSIIIRMRDVVLFEKKVYLSECKTGNGKNYRGTMSKTKNGITCQKWSSTSPHRPRFSPATHPSEGLEENYCRNPDNDPQGPWCYTTDPEKRYDYCDILECEEECMHCSGENYDGKISKTMSGLECQAWDSQSPHAHGYIPSKFPNKNLKKNYCRNPDRELRPWCFTTDPNKRWELCDIPRCTTPPPSSGPTYQCLKGTGENYRGNVAVTVSGHTCQHWSAQTPHTHNRTPENFPCKNLDENYCRNPDGKRAPWCHTTNSQVRWEYCKIPSCDSSPVSTEQLAPTAPPELTPVVQDCYHGDGQSYRGTSSTTTTGKKCQSWSSMTPHRHQKTPENYPNAGLTMNYCRNPDADKGPWCFTTDPSVRWEYCNLKKCSGTEASVVAPPPVVLLPDVETPSEEDCMFGNGKGYRGKRATTVTGTPCQDWAAQEPHRHSIFTPETNPRAGLEKNYCRNPDGDVGGPWCYTTNPRKLYDYCDVPQCAAPSFDCGKPQVEPKKCPGRVVGGCVAHPHSWPWQVSLRTRFGMHFCGGTLISPEWVLTAAHCLEKSPRPSSYKVILGAHQEVNLEPHVQEIEVSRLFLEPTRKDIALLKLSSPAVITDKVIPACLPSPNYVVADRTECFITGWGETQGTFGAGLLKEAQLPVIENKVCNRYEFLNGRVQSTELCAGHLAGGTDSCQGDSGGPLVCFEKDKYILQGVTSWGLGCARPNKPGVYVRVSRFVTWIEGVMRNN

SEQ ID NO: 17 - DNA, 2433 bp, Homo sapiens
  CDS (1)...(2433): Encodes D472N V701I variant of full-length human plasminogen of SEQ ID NO2
atggaacataaggaagtggttcttctacttcttttatttctgaaatcaggtcaaggagagcctctggatgactatgtgaatacccagggggcttcactgttcagtgtcactaagaagcagctgggagcaggaagtatagaagaatgtgcagcaaaatgtgaggaggacgaagaattcacctgcagggcattccaatatcacagtaaagagcaacaatgtgtgataatggctgaaaacaggaagtcctccataatcattaggatgagagatgtagttttatttgaaaagaaagtgtatctctcagagtgcaagactgggaatggaaagaattacagagggacgatgtccaaaacaaaaaatggcatcacctgtcaaaaatggagttccacttctccccacagacctagattctcacctgctacacacccctcagagggactggaggagaactactgcaggaatccagacaacgatccgcaggggccctggtgctatactactgatccagaaaagagatatgactactgcgacattcttgagtgtgaagaggaatgtatgcattgcagtggagaaaactatgacggcaaaatttccaagaccatgtctggactggaatgccaggcctgggactctcagagcccacacgctcatggatacattccttccaaatttccaaacaagaacctgaagaagaattactgtcgtaaccccgatagggagctgcggccttggtgtttcaccaccgaccccaacaagcgctgggaactttgcgacatcccccgctgcacaacacctccaccatcttctggtcccacctaccagtgtctgaagggaacaggtgaaaactatcgcgggaatgtggctgttaccgtgtccgggcacacctgtcagcactggagtgcacagacccctcacacacataacaggacaccagaaaactttccctgcaaaaatttggatgaaaactactgccgcaatcctgacggaaaaagggccccatggtgccatacaaccaacagccaagtgcggtgggagtactgtaagataccgtcctgtgactcctccccagtatccacggaacaattggctcccacagcaccacctgagctaacccctgtggtccaggactgctaccatggtgatggacagagctaccgaggcacatcctccaccaccaccacaggaaagaagtgtcagtcttggtcatctatgacaccacaccggcaccagaagaccccagaaaactacccaaatgctggcctgacaatgaactactgcaggaatccagatgccgataaaggcccctggtgttttaccacagaccccagcgtcaggtgggagtactgcaacctgaaaaaatgctcaggaacagaagcgagtgttgtagcacctccgcctgttgtcctgcttccaaatgtagagactccttccgaagaagactgtatgtttgggaatgggaaaggataccgaggcaagagggcgaccactgttactgggacgccatgccaggactgggctgcccaggagccccatagacacagcattttcactccagagacaaatccacgggcgggtctggaaaaaaattactgccgtaaccctgatggtgatgtaggtggtccctggtgctacacgacaaatccaagaaaactttacgactactgtgatgtccctcagtgtgcggccccttcatttgattgtgggaagcctcaagtggagccgaagaaatgtcctggaagggttgtaggggggtgtgtggcccacccacattcctggccctggcaagtcagtcttagaacaaggtttggaatgcacttctgtggaggcaccttgatatccccagagtgggtgttgactgctgcccactgcttggagaagtccccaaggccttcatcctacaaggtcatcctgggtgcacaccaagaagtgaatctcgaaccgcatgttcaggaaatagaagtgtctaggctgttcttggagcccacacgaaaagatattgccttgctaaagctaagcagtcctgccgtcatcactgacaaagtaatcccagcttgtctgccatccccaaattatgtggtcgctgaccggaccgaatgtttcatcactggctggggagaaacccaaggtacttttggagctggccttctcaaggaagcccagctccctgtgattgagaataaagtgtgcaatcgctatgagtttctgaatggaagagtccaatccaccgaactctgtgctgggcatttggccggaggcactgacagttgccagggtgacagtggagggcctctggtttgcttcgagaaggacaaatacattttacaaggagtcacttcttggggtcttggctgtgcacgccccaataagcctggtgtctatgttcgtgtttcaaggtttgttacttggattgagggagtgatgagaaataattaa

SEQ ID NO: 18 - protein, 810 aa, Homo sapiens [sequence omitted]

SEQ ID NO: 19 - DNA, 2376 bp, Homo sapiens
  CDS (1)...(2376): Encodes D453N V682I variant of mature human plasminogen of SEQ ID NO4
gagcctctggatgactatgtgaatacccagggggcttcactgttcagtgtcactaagaagcagctgggagcaggaagtatagaagaatgtgcagcaaaatgtgaggaggacgaagaattcacctgcagggcattccaatatcacagtaaagagcaacaatgtgtgataatggctgaaaacaggaagtcctccataatcattaggatgagagatgtagttttatttgaaaagaaagtgtatctctcagagtgcaagactgggaatggaaagaattacagagggacgatgtccaaaacaaaaaatggcatcacctgtcaaaaatggagttccacttctccccacagacctagattctcacctgctacacacccctcagagggactggaggagaactactgcaggaatccagacaacgatccgcaggggccctggtgctatactactgatccagaaaagagatatgactactgcgacattcttgagtgtgaagaggaatgtatgcattgcagtggagaaaactatgacggcaaaatttccaagaccatgtctggactggaatgccaggcctgggactctcagagcccacacgctcatggatacattccttccaaatttccaaacaagaacctgaagaagaattactgtcgtaaccccgatagggagctgcggccttggtgtttcaccaccgaccccaacaagcgctgggaactttgcgacatcccccgctgcacaacacctccaccatcttctggtcccacctaccagtgtctgaagggaacaggtgaaaactatcgcgggaatgtggctgttaccgtgtccgggcacacctgtcagcactggagtgcacagacccctcacacacataacaggacaccagaaaactttccctgcaaaaatttggatgaaaactactgccgcaatcctgacggaaaaagggccccatggtgccatacaaccaacagccaagtgcggtgggagtactgtaagataccgtcctgtgactcctccccagtatccacggaacaattggctcccacagcaccacctgagctaacccctgtggtccaggactgctaccatggtgatggacagagctaccgaggcacatcctccaccaccaccacaggaaagaagtgtcagtcttggtcatctatgacaccacaccggcaccagaagaccccagaaaactacccaaatgctggcctgacaatgaactactgcaggaatccagatgccgataaaggcccctggtgttttaccacagaccccagcgtcaggtgggagtactgcaacctgaaaaaatgctcaggaacagaagcgagtgttgtagcacctccgcctgttgtcctgcttccaaatgtagagactccttccgaagaagactgtatgtttgggaatgggaaaggataccgaggcaagagggcgaccactgttactgggacgccatgccaggactgggctgcccaggagccccatagacacagcattttcactccagagacaaatccacgggcgggtctggaaaaaaattactgccgtaaccctgatggtgatgtaggtggtccctggtgctacacgacaaatccaagaaaactttacgactactgtgatgtccctcagtgtgcggccccttcatttgattgtgggaagcctcaagtggagccgaagaaatgtcctggaagggttgtaggggggtgtgtggcccacccacattcctggccctggcaagtcagtcttagaacaaggtttggaatgcacttctgtggaggcaccttgatatccccagagtgggtgttgactgctgcccactgcttggagaagtccccaaggccttcatcctacaaggtcatcctgggtgcacaccaagaagtgaatctcgaaccgcatgttcaggaaatagaagtgtctaggctgttcttggagcccacacgaaaagatattgccttgctaaagctaagcagtcctgccgtcatcactgacaaagtaatcccagcttgtctgccatccccaaattatgtggtcgctgaccggaccgaatgtttcatcactggctggggagaaacccaaggtacttttggagctggccttctcaaggaagcccagctccctgtgattgagaataaagtgtgcaatcgctatgagtttctgaatggaagagtccaatccaccgaactctgtgctgggcatttggccggaggcactgacagttgccagggtgacagtggagggcctctggtttgcttcgagaaggacaaatacattttacaaggagtcacttcttggggtcttggctgtgcacgccccaataagcctggtgtctatgttcgtgtttcaaggtttgttacttggattgagggagtgatgagaaataattaa

SEQ ID NO: 20 - protein, 791 aa, Homo sapiens
  VARIANT (1)...(791): Sequence of D453N V682I variant of mature human plasminogen of SEQ ID NO4
EPLDDYVNTQGASLFSVTKKQLGAGSIEECAAKCEEDEEFTCRAFQYHSKEQQCVIMAENRKSSIIIRMRDVVLFEKKVYLSECKTGNGKNYRGTMSKTKNGITCQKWSSTSPHRPRFSPATHPSEGLEENYCRNPDNDPQGPWCYTTDPEKRYDYCDILECEEECMHCSGENYDGKISKTMSGLECQAWDSQSPHAHGYIPSKFPNKNLKKNYCRNPDRELRPWCFTTDPNKRWELCDIPRCTTPPPSSGPTYQCLKGTGENYRGNVAVTVSGHTCQHWSAQTPHTHNRTPENFPCKNLDENYCRNPDGKRAPWCHTTNSQVRWEYCKIPSCDSSPVSTEQLAPTAPPELTPVVQDCYHGDGQSYRGTSSTTTTGKKCQSWSSMTPHRHQKTPENYPNAGLTMNYCRNPDADKGPWCFTTDPSVRWEYCNLKKCSGTEASVVAPPPVVLLPNVETPSEEDCMFGNGKGYRGKRATTVTGTPCQDWAAQEPHRHSIFTPETNPRAGLEKNYCRNPDGDVGGPWCYTTNPRKLYDYCDVPQCAAPSFDCGKPQVEPKKCPGRVVGGCVAHPHSWPWQVSLRTRFGMHFCGGTLISPEWVLTAAHCLEKSPRPSSYKVILGAHQEVNLEPHVQEIEVSRLFLEPTRKDIALLKLSSPAVITDKVIPACLPSPNYVVADRTECFITGWGETQGTFGAGLLKEAQLPVIENKVCNRYEFLNGRVQSTELCAGHLAGGTDSCQGDSGGPLVCFEKDKYILQGVTSWGLGCARPNKPGVYVRVSRFVTWIEGVMRNN

(19) United States
(12) Patent Application Publication
(10) Pub. No.: US 2012/0220015 A1
(43) Pub. Date: Aug. 30, 2012

(54) COMPOSITIONS AND METHODS FOR PRODUCTION OF AGLYCOSYLATED PLASMINOGEN

(75) Inventors: Lynn F. Dickey, Cary, NC (US); John R. Gasdaska, Carrboro, NC (US); Kevin M. Cox, Raleigh, NC (US)

(73) Assignee: BIOLEX THERAPEUTICS, Pittsboro, NC (US)

(21) Appl. No.: 13/321,719

(22) PCT Filed: May 26, 2010
(86) PCT No.: PCT/US10/36253

§ 371 Date: Dec. 6, 2011

(30) Foreign Application Priority Data
May 26, 2009 (US) 61181170

(51) Int. Cl. C12N 9/68; C12N 5/10; A01H 5/00; C12N 15/57; C12N 15/82

(52) U.S. Cl. 435/217; 536/23.2; 435/320.1; 800/298; 435/419

(57) ABSTRACT

Compositions and methods for producing aglycosylated plasminogen (PLG) polypeptides and fragments and variants thereof are provided. Compositions of the invention include isolated nucleic acid molecules encoding aglycosylated PLG polypeptides in which the asparagine (Asn) residue corresponding to residue Asn-289 of the mature human PLG polypeptide has been substituted with an amino acid residue that does not support N-linked glycosylation at that position of the PLG polypeptide, as well as the aglycosylated PLG polypeptides encoded thereby. Expression constructs comprising these PLG-encoding nucleic acid molecules and transgenic plants comprising these expression constructs are also provided. Methods of the invention comprise introducing a PLG-encoding nucleic acid molecule of the invention into a plant of interest and culturing the plant under conditions to produce the aglycosylated PLG polypeptide. The aglycosylated PLG polypeptide allows for significant increases in production and yield of PLG from a plant-based expression system without comprising the ability of the PLG product to be activated to a polypeptide capable of binding fibrin and having serine protease activity, including biologically active plasmin that is also glycosylated. The activated aglycosylated plasmin is useful to treat diseases or conditions associated with a thrombus.

### FIELD OF THE INVENTION

The invention relates generally to compositions and methods for recombinant production of plasminogen (PLG), and more particularly to compositions and methods for producing aglycosylated PLG in plants.

### BACKGROUND

In humans, plasminogen (PLG) is a single-chain, inactive glycoprotein of 791 amino acid residues and is encoded by the PLG gene. Several forms of PLG in plasma are known and can be separated by affinity chromatography. The native form of human PLG in plasma has glutamic acid at the N-terminus (Forsgren et al. (1987) FEBS Lett. 213:254-260; Malinowski et al. (1984) Biochem. 23: 4243-4250; McLean et al. (1987) Nature 330:132-137; Sottrup-Jensen et al. (1978) Prog. Chem. Fibrinolysis Thrombolysis 3:191-209; Wiman (1973) Eur. J. Biochem. 39:1-9; and Wiman (1977) Eur. J. Biochem. 76:129-137).

Human PLG exists as two major glycosylation variants, type 1 (about 33%) and type 2 (about 67%) in circulation (Pirie-Shepherd (1999) J. Lab. Clin. Med. 134:553-560). The two types differ only in their carbohydrate content (Hayes and Castellino (1979) J. Biol. Chem. 254:8768-8771; Davidson and Castellino (1991) Biochemistry 30:625-633). Type 1 glycoform of human PLG contains one N-linked glycosylation at residue Asn-289 and one O-linked glycosylation at residue Thr-346 (Davidson and Castellino (1991) Biochemistry 30:625-633; Pirie-Shepherd et al. (1997) J. Biol. Chem. 272:7408-7411), where residues are numbered according to the 791-residue mature protein sequence. Type 2 glycoform of human PLG contains only O-linked glycosylation at Ser-249 and Thr-346 of the mature sequence (Davidson and Castellino (1991) Biochemistry 30:625-633; Pirie-Shepherd et al. (1997) J. Biol. Chem. 272:7408-7411). The structure of these N-linked and O-linked oligosaccharide units has been previously characterized (see, for example, Hayes and Castellino (1979) J. Biol. Chem. 254:8772-8776, and 8777-8780; and Pirie-Shepherd et al. (1997) J. Biol. Chem. 272:7408-7411).

Plasminogen, the precursor of plasmin, is a circulating zymogen lacking protease activity. It is activated to plasmin by activators such as streptokinase, tissue plasminogen activator (tPA), or urokinase. Upon processing and activation, two significant molecular events occur. First, a proactive peptide is removed from the N-terminus; second, an internal cleavage of the peptide bond occurring between residue Arg-561 and Val-562 of PLG results in the formation of plasmin.

Plasmin is a serine protease involved in the fibrinolytic system. It also may be involved in cell migration, tissue remodeling, and bacterial invasion. Plasmin is a heterodimer, the two chains of which are held together by two interchain disulphide bridges. The light chain (25 kDa), which consists of the C-terminal 230 amino acids of plasminogen, carries a catalytic center that comprises a catalytic triad and shares sequence similarity with trypsin and other serine proteases. The heavy chain (60 kDa) consists of five highly similar triple-loop structures called kringles. Some of the kringles contain Lys binding sites that mediate the PLG/plasmin interaction with fibrin(ogen). Plasmin belongs to peptidase family S1 and preferentially cleaves Lys-|-Xaa and Arg-|-Xaa bonds with higher selectivity than trypsin. Plasmin is inactivated by inactivators such as α₂-antiplasmin (α₂-AP) or α₂-macroglobulin (α₂-M). Deficiency in plasmin may lead to thrombosis, as fibrin clots are not degraded adequately.

Microplasminogen (mPLG) consists of the proenzyme domain of PLG with a stretch of connecting peptide and a few residues of kringle 5 attached at its N-terminal end. It is produced by the action of plasmin on PLG. Like PLG, mPLG is stable (i.e., inactive). See, e.g., Shi and Wu (1988) J. Biol. Chem. 263:17071-17075. mPLG can be activated by activators such as tPA and urokinase to form a proteolytically active molecule, microplasmin. Human microplasmin has a molecular weight of approximately 29 kDa and has a lower affinity for fibrin(ogen) when compared with plasmin.

Given their involvement in the fibrinolytic system, plasmin and microplasmin are proposed for use in thrombolytic therapy in a number of applications including, but not limited to, treatment of myocardial infarction, occlusive stroke, hemodialysis graft thrombosis, deep venous thrombosis, and peripheral arterial diseases, including acute peripheral arterial occlusion. See, for example, U.S. Pat. Nos. 5,407,673 and 6,355,243; U.S. Patent Application Publication No. 2003/0175264; Lapchak et al. (2002) Stroke 33:2279-2284; and Nagai et al. (2003) J. Thromb. Haemost. 1:307-313, each of which is incorporated herein by reference as if set forth in its entirety. One goal of using plasmin and microplasmin in such therapy is to avoid side effects of activators such as streptokinase, tPA, or urokinase, as these activators can cause gastrointestinal and intercranial hemorrhage. However, the use of plasmin and microplasmin as therapeutic agents has been limited in part by a difficulty of producing large quantities of stable (i.e., inactive) PLG precursor.

Although expression systems can be a convenient way to obtain large quantities of PLG for use in thrombolytic therapy, there have been great difficulties in expressing stable (i.e., inactive) human PLG because of a nearly ubiquitous presence of intracellular PLG activators in mammalian host cells. These activators result in the degradation of PLG. See, e.g., Busby et al. (1988) Fibrinolysis 2 (Supp 1):64; and Busby et al. (1991) J. Biol. Chem. 266:15286-15292. Recombinant PLG has been produced in other eukaryotic cells, such as insect and plant cells. See, for example, Whitefleet-Smith et al. (1989) Arch. Biochem. Biophys. 271:390-399; Nilsen and Castellino (1999) Protein Expr. Pur 16:136-143; and U.S. Patent Application Publication No. 2005/0262592. See also Browne et al. (1991) Fibrinolysis 5:257-260, describing the expression of recombinant human plasminogen and aglyco (Asn289Gln, Thr346Ala) plasminogen in HeLa cells.

Of particular interest herein are plant-based gene expression systems for production of PLG. Plant-based gene expression systems provide a pivotal technology for a number of research and commercial applications. A differentiated plant-based expression system that can be manipulated with the laboratory convenience of yeast provides a very fast system in which to analyze the developmental and physiological roles of isolated genes. Moreover, and for commercial production of valuable polypeptides such as PLG, a plant-based expression system has a number of advantages over existing microbial or other cell culture systems.

Plants demonstrate post-translational processing that is similar to mammalian cells, overcoming one major problem associated with the microbial cell production of biologically active, mammalian polypeptides, and it has been shown by others (Hiatt (1990) Nature 344:469-470) that plant expression systems have an ability to assemble multi-subunit proteins, which is often lacking in microbial expression systems. Scale-up of plant biomass to levels necessary for commercial production of recombinant proteins is faster and more cost efficient than similar scale-up of mammalian expression systems.

For the foregoing reasons, there remains a need for optimized compositions for expressing PLG in plant-based expression systems.

### BRIEF SUMMARY

The present invention provides compositions and methods relating to plasminogen (PLG), more particularly aglycosylated PLG polypeptides produced by plant-based gene expression systems. Compositions of the invention include isolated nucleic acid molecules encoding aglycosylated PLG polypeptides, for example, an aglycosylated form of human PLG or aglycosylated variant thereof, in which the asparagine (Asn) residue corresponding to residue Asn-289 of the mature human PLG polypeptide has been substituted with an amino acid residue that does not support N-linked glycosylation at that position of the PLG polypeptide. In this manner, the isolated nucleic acid molecules of the invention encode aglycosylated PLG polypeptides, wherein the codon for the amino acid residue corresponding to Asn-289 of mature human PLG has been modified to encode an amino acid that cannot be glycosylated by attachment of an N-linked glycan. By modifying this codon to encode for a residue that prevents N-linked glycosylation of the expressed PLG polypeptide, it is possible to significantly increase production and yield of recombinantly produced PLG from a plant-based expression system without compromising the ability of the recombinant PLG product to be activated to biologically active plasmin, which also retains the aglycosylated feature of the PLG polypeptide from which it is derived.

Expression constructs (for example, expression cassettes and expression vectors) comprising the PLG-encoding nucleic acid molecules of the invention are also provided for use in recombinant production of the encoded aglycosylated PLG polypeptides in a plant host of interest. The PLG-encoding nucleic acid molecules of the invention can be codon-optimized for expression in the plant host of interest.

The present invention thus provides methods and compositions for the production of aglycosylated PLG polypeptides in a plant-based expression system, plants that are transformed with expression constructs comprising the PLG-encoding nucleic acid molecules of the invention, and compositions comprising the isolated aglycosylated PLG polypeptides of the invention. Thus, in one embodiment, the present invention provides a method for producing aglycosylated PLG polypeptides in a plant-based expression system, wherein the method comprises introducing into the plant host of interest a nucleic acid molecule encoding an aglycosylated PLG polypeptide described herein, and culturing the transformed plant under conditions suitable for production of the aglycosylated PLG polypeptide. The recombinantly produced aglycosylated PLG polypeptide can then be collected from the transformed plant or plant part thereof, or, where expressed with a signal peptide, collected from one or both of the transformed plant or plant part thereof and the plant culture medium. The aglycosylated PLG polypeptides produced in the plant host of interest are stable (i.e., inactive), but can be activated to a polypeptide having serine protease activity (for example, plasmin). In this manner, the aglycosylated PLG polypeptides of the invention can be activated to plasmin. Thus, the present invention also provides compositions, including pharmaceutical compositions, comprising plasmin derived from the aglycosylated PLG polypeptides of the present invention. The derived plasmin retains the aglycosylated feature of the aglycosylated PLG polypeptides of the present invention.

The following embodiments are encompassed by the invention:

1. An isolated nucleic acid molecule comprising a nucleotide sequence encoding a plasminogen (PLG) polypeptide having at least 95% amino acid sequence identity to the sequence set forth in SEQ ID NO:4, wherein said PLG polypeptide comprises an amino acid residue other than asparagine (Asn) at the residue position corresponding to Asn-289 of SEQ ID NO:4, and wherein said PLG polypeptide retains an O-linked glycosylation site at the residue position corresponding to threonine-346 (Thr-346) of SEQ ID NO:4, wherein said PLG polypeptide is capable of being activated to a polypeptide having serine protease activity.

2. The isolated nucleic acid molecule of embodiment 1, wherein said PLG polypeptide comprises a Thr residue at the residue position corresponding to Thr-346 of SEQ ID NO:4.

3. The isolated nucleic acid molecule of embodiment 1 or 2, wherein the amino acid residue at the residue position corresponding to Asn-289 of SEQ ID NO:4 is selected from the group consisting of glutamine (Gln), histidine (His), lysine (Lys), Arginine (Arg), and aspartic acid (Asp).

4. The isolated nucleic acid molecule of embodiment 3, wherein said PLG polypeptide comprises the amino acid sequence set forth in SEQ ID NO:4 with a Gln, His, Lys, Arg, or Asp residue substituted for Asn-289 of SEQ ID NO:4.

5. The isolated nucleic acid molecule of embodiment 4, wherein said PLG polypeptide comprises the amino acid sequence set forth in SEQ ID NO:6.

6. The isolated nucleic acid molecule of any one of embodiments 1-5, wherein said nucleotide sequence encoding said PLG polypeptide is codon-optimized for expression in a plant of interest.

7. The isolated nucleic acid molecule of embodiment 6, wherein said nucleotide sequence encoding said PLG polypeptide comprises at least 70% plant-preferred codons.

8. The isolated nucleic acid molecule of embodiment 7, wherein said nucleotide sequence encoding said PLG polypeptide comprises the sequence set forth in SEQ ID NO:5.

9. An isolated nucleic acid molecule comprising a nucleotide sequence encoding a plasminogen (PLG) polypeptide having at least 95% amino acid sequence identity to the sequence set forth in SEQ ID NO:4, wherein said PLG polypeptide comprises an amino acid residue other than asparagine (Asn) at the residue position corresponding to Asn-289 of SEQ ID NO:4, and wherein said PLG polypeptide is capable of being activated to a polypeptide having serine protease activity, wherein said nucleotide sequence encoding said PLG polypeptide is codon-optimized for expression in a plant of interest.

10. The isolated nucleic acid molecule of embodiment 9, wherein the amino acid residue at the residue position corresponding to Asn-289 of SEQ ID NO:4 is selected from the group consisting of glutamine (Gln), histidine (His), lysine (Lys), Arginine (Arg), and aspartic acid (Asp).

11. The isolated nucleic acid molecule of embodiment 10, wherein said PLG polypeptide comprises the amino acid sequence set forth in SEQ ID NO:4 with a Gln, His, Lys, Arg, or Asp residue substituted for Asn-289 of SEQ ID NO:4.

12. The isolated nucleic acid molecule of embodiment 11, wherein said PLG polypeptide comprises the amino acid sequence set forth in SEQ ID NO:6.

13. The isolated nucleic acid molecule of any one of embodiments 9-12, wherein said nucleotide sequence encoding said PLG polypeptide comprises at least 70% plant-preferred codons.

14. The isolated nucleic acid molecule of embodiment 13, wherein said nucleotide sequence encoding said PLG polypeptide comprises the sequence set forth in SEQ ID NO:5.

15. An expression construct comprising the nucleic acid molecule of any one of embodiments 1-14.

16. The expression construct of embodiment 15, wherein said nucleotide sequence encoding said PLG polypeptide is operably linked to a nucleotide sequence encoding a signal peptide that directs secretion of said PLG polypeptide.

17. The expression construct of embodiment 16, wherein said nucleotide sequence encoding said signal peptide is selected from the group consisting of:
a) the sequence set forth in SEQ ID NO:7;
b) a sequence having at least 90% sequence identity to SEQ ID NO:7, wherein said sequence encodes a signal peptide that directs secretion of said PLG polypeptide; and
c) a sequence comprising a functional fragment of SEQ ID NO:7, wherein said sequence directs secretion of said PLG polypeptide.

18. The expression construct of any one of embodiments 15-17, wherein said nucleotide sequence encoding said PLG polypeptide is operably linked to a nucleotide sequence comprising a plant intron that is inserted upstream of said nucleotide sequence encoding said PLG polypeptide.

19. The expression construct of embodiment 18, wherein said plant intron is an intron from the maize alcohol dehydrogenase 1 gene.

20. The expression construct of embodiment 19 wherein said plant intron comprises the sequence set forth in SEQ ID NO:9.

21. The expression construct of any one of embodiments 15-20, wherein said nucleotide sequence encoding said PLG polypeptide is operably linked to a nucleotide sequence comprising a translation leader sequence.

22. The expression construct of embodiment 21, wherein said translation leader sequence is from a ribulose-bis-phosphate carboxylase small subunit 5B gene of Lemna gibba.

23. The expression construct of embodiment 22, wherein said translation leader sequence comprises SEQ ID NO:10.

24. A transformed plant or plant cell or plant nodule comprising the expression construct of any one of embodiments 15-23.

25. The transformed plant or plant cell or plant nodule of embodiment 24, wherein said plant is a monocot, or wherein said plant cell or plant nodule is from a monocot.

26. The transformed plant or plant cell or plant nodule of embodiment 25, wherein the monocot is from a genus selected from the group consisting of the genus Spirodela, genus Wolffia, genus Wolfiella, genus Landoltia and genus Lemna.

27. The transformed plant or plant cell or plant nodule of embodiment 26, wherein said monocot is a member of a species selected from the group consisting of Lemna minor, Lemna miniscula, Lemna aequinoctialis and Lemna gibba.

28. A method for producing an aglycosylated plasminogen (PLG) polypeptide in a plant, the method comprising the steps of:
a) culturing in a culture medium under conditions suitable for expression of said PLG polypeptide a plant, plant cell, or plant nodule comprising an expression construct of any one of embodiments 15-23; and
b) collecting said PLG polypeptide from at least one of said culture medium, plant, plant cell, or plant nodule.

29. The method of embodiment 28, wherein said plant is a monocot or wherein said plant cell or plant nodule is from a monocot.

30. The method of embodiment 29, wherein said monocot is from a genus selected from the group consisting of the genus Spirodela, genus Wolffia, genus Wolfiella, genus Landoltia and genus Lemna.

31. The method of embodiment 30, wherein said monocot is a member of a species selected from the group consisting of Lemna minor, Lemna miniscula, Lemna aequinoctialis and Lemna gibba.

32. An isolated aglycosylated plasminogen (PLG) polypeptide comprising an amino acid sequence having at least 95% sequence identity to the sequence set forth in SEQ ID NO:4, wherein the asparagine (Asn) residue at the position corresponding to position 289 of SEQ ID NO:4 has been substituted with an amino acid residue other than Asn, and wherein said PLG polypeptide retains an O-linked glycosylation site at the residue position corresponding to threonine-346 (Thr-346) of SEQ ID NO:4, wherein said PLG polypeptide is capable of being activated to a polypeptide having serine protease activity.

33. The isolated aglycosylated PLG polypeptide of embodiment 32, wherein the amino acid residue at the position corresponding to position 346 of SEQ ID NO:4 is threonine (Thr).

34. The isolated aglycosylated PLG polypeptide of embodiment 32 or 33, wherein the amino acid residue at the position corresponding to position 289 of SEQ ID NO:4 is selected from the group consisting of glutamine (Gln), histidine (His), lysine (Lys), Arginine (Arg), and aspartic acid (Asp).

35. The isolated aglycosylated PLG polypeptide of embodiment 34, wherein said PLG comprises the amino acid sequence set forth in SEQ ID NO:4, wherein the amino acid at position 289 of SEQ ID NO:4 is substituted with an amino acid residue selected from the group consisting of Gln, His, Lys, Arg, and Asp.

36. The isolated aglycosylated PLG polypeptide of embodiment 35, wherein said PLG comprises the amino acid sequence set forth in SEQ ID NO:6.

37. Aglycosylated plasmin derived from the aglycosylated PLG polypeptide of any one of embodiments 32-36.

38. A composition comprising the aglycosylated plasmin of embodiment 37.

39. The composition of embodiment 38, wherein said composition is a pharmaceutical composition.

40. A method for the treatment of diseases or conditions associated with a thrombus, said method comprising the administration of a therapeutically effective amount of the composition of claim 39.

41. Use of the composition of embodiment 39 for the treatment of diseases or conditions associated with a thrombus.

### BRIEF DESCRIPTION OF THE DRAWINGS

FIG. 1A shows a Southern Blot to characterize the t-DNA insertion pattern for the transgenic line BAP12-B2-150, which expresses mature human PLG with an N289D substitution (i.e., an aspartic acid (D) residue has been substituted for the asparagine (N) residue at position 289 of mature human PLG; see SEQ ID NO:6). WT corresponds to wildtype genomic DNA from Lemna minor 8627. (+) control corresponds to WT genomic DNA spiked with BAP12-B2-150 plasmid DNA (FIG. 1B).

FIGS. 2A-2H show nucleic acid sequence alignments between the five cDNA clones from BAP12 (encoding mature human PLG with the N289D substitution). The expected Lemna-derived PLG coding sequence (SEQ ID NO:5) is denoted by “BAP12 VNTI” (Vector NTI). The consensus of all of the cDNA sequences is given on the last line of the alignment. All cDNA sequences generated from the high-expressing transgenic line BAP12-B2-150 are 100% identical to the PLG reference sequence, and thus all correspond to the sequence set forth in SEQ ID NO:5.

FIGS. 3A and 3B show growth of transgenic line BAP12-B2-150 and PLG specific activity, respectively. FIG. 3A shows fresh weight of tissue (in grams) after 16 days and 18 days of growth of transgenic line BAP12-B2-150 in vented research vessels (referred to as IVs). Total PLG accumulation in the tissue for line BAP12-B2-150 (as measured by plasmin (PLM) activity assay) was between 400 and 680 μg per gram fresh weight of tissue.

FIG. 4 shows a Western blot of PLG crude extract from various BAP12-expressing plant lines.

FIG. 5 shows an SDS-PAGE of non-reduced lysine sepharose-purified PLG.

FIG. 6 shows a non-reducing SDS-PAGE of Lemna-derived plasmin from transgenic line BAP12-B2-150 (LPlm-Line 150) analyzed by SDS-PAGE along side plasmin from transgenic line 230 (LPlm-Line 230) and human reference plasmin (HPlm). The source of the proteins as well as the amount loaded per well are indicated on the top of the gels. Non-reduced and reduced analyses are shown on the left and right panels, respectively. Line 230 refers to BAP-01-B2-230 (transgenic line expressing mature human plasminogen of SEQ ID NO:4, which has the N-linked glycosylation site Asn-289); Line 150 refers to BAP12-B2-150 (transgenic line expressing mature human plasminogen of SEQ ID NO:6 having the N289D substitution); and HPlm refers to commercial, human-derived plasmin.

FIG. 7 shows representative size-exclusion chromatography (SEC) chromatograms from Lemna-derived plasmin from line BAP12-B2-150 and human reference plasmin. 25 μg of sample was analyzed with each run.

FIG. 8 shows SDS-PAGE of plasmin:α₂-AP mixtures that reveal inactivation of plasmin by a complex formation with α₂-antiplasmin (α₂-AP).

FIGS. 9A and 9B show inhibition profiles of human and Lemna-derived plasmin by α₂-AP. In FIG. 9A, plasmin and α₂-AP were pre-incubated for 30 minutes before estimating the concentration of active plasmin. The assay consisted of mixing 50 μl of plasmin at 10 μg/ml with 50 μl of increasing concentrations of α₂-AP in a 96 well plate. The molar ratios are indicated on the x-axis. Before reading at absorbance 405 nm, 50 μl of chromogenic substrate S-2403 was added. In FIG. 9B, plasmin and α₂-AP were not pre-incubated before monitoring the plasmin activity. For this design, α₂-AP was mixed with the substrate S-2403, and plasmin was added last, right before starting the analysis of the reaction.

FIG. 10 shows lysis profiles of fibrin clots comparing human reference plasmin and plasmin from transgenic lines BAP01-B2-230 and BAP12-B2-150.

FIG. 11 shows a diode array chromatogram comparison of human plasmin, BAP01-B2-230 plasmin, and BAP12-B2-150 plasmin.

FIG. 12 shows a zoom of the chromatogram shown in the previous figure, highlighting the peaks uncommon to the plasmin derived from the two transgenic plant lines BAP12-B2-150 and BAP01-B2-230. The three peaks were identified as peptide H16, in which the Asn residue in BAP01-B2-230 was changed to Asp in BAP12-B2-150. In line BAP01-B2-230, the Asn residue was partly glycosylated. The substituted Asp residue in line BAP12-B2-150 was not glycosylated.

FIG. 13 shows plasmin activity analysis of individual peaks from the C18 RP separation of transgenic line BAP12-B2-150. The majority of the activity was observed in peaks #4 and #5 from the RP chromatogram.

FIG. 14 shows a reduced SDS-PAGE analysis of individual peaks from the C18 RP separation of transgenic line BAP12-B2-150. The majority of the full-length plasmin was observed in peaks #4 and #5 as the 31 kD and 66 kD proteins from the RP chromatogram.

FIG. 15 shows overlays of SEC chromatograms for human reference plasmin and plasmin derived from transgenic line BAP12-B2-150 after three cycles of freeze-thaw.

FIG. 16 shows a summary of plasmin activity for plasmin derived from transgenic line BAP12-B2-150 upon storage at ambient temperature and at 4° C.

FIG. 17 shows overlays of SEC chromatograms obtained after incubation at ambient temperature and at 4° C. for plasmin derived from transgenic line BAP12-B2-150.

FIG. 18 shows a comparison of yield of mature plasminogen (PLG) (mgs PLG per kilo (kg) fresh weight of tissue) from transgenic duckweed lines expressing the glycosylated form of human PLG (BAP01-B2-230) and the aglycosylated form of human PLG (BAP12-B2-150). Key: A=Line BAP01-B2-230, cultured 21 days in passive vented SV at 24.5° C. B=Line BAP01-B2-230, cultured 28 days in passive vented SV at 24.5° C. C=Line BAP12-B2-150, cultured for 28 days in passive vented SV at 24.5° C. D=Line BAP12-B2-150, cultured for 21 days in a vented FASV at 21° C. E=Line BAP12-B2-150, cultured for 33 days in a vented FASV at 21° C.

FIG. 19 shows a comparison of yield of mature plasminogen (PLG) (mgs PLG per growth vessel) from transgenic duckweed lines expressing the glycosylated form of human PLG (BAP01-B2-230) and the aglycosylated form of human PLG (BAP12-B2-150). Key: A=Line BAP01-B2-230, cultured 21 days in passive vented SV at 24.5° C. (fresh weight, 130 g). B=Line BAP01-B2-230, cultured 28 days in passive vented SV at 24.5° C. (fresh weight, 138 g). C=Line BAP12-B2-150, cultured for 28 days in passive vented SV at 24.5° C. (fresh weight, 86 g). D=Line BAP12-B2-150, cultured for 21 days in a vented FASV at 21° C. (fresh weight, 71 g). E=Line BAP12-B2-150, cultured for 33 days in a vented FASV at 21° C. (fresh weight, 106 g).

FIG. 20A shows the common oligomannosidic core structure of complex N-glycans of glycoproteins produced in plants and animals. In mammals, the core structure can include a fucose residue in which 1-position of the fucose is bound to 6-position of the N-acetylgucosamine in the reducing end through an a bond (i.e., α(1,6)-linked fucose). FIG. 20B shows the plant-specific modifications to these N-glycans. The mammalian R groups can be one of the following: (a) R=GlcNAcβ(1,2); (b) R=Galβ(1,4)-GlcNAcβ(1,2); (c) R=NeuAcα(2,3)-Galβ(1,4)-GlcNAcβ(1,2); (d) R=NeuGcα(2,3)-Galβ(1,4)-GlcNAcβ(1,2); and (e) R=Galα(1,3)-Galβ(1,4)-GlcNAcβ(1,2). The plant R groups can be one of the following: (a) R=null; (b) R=GlcNAcβ(1,2);

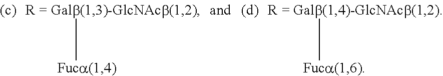

Abbreviations: Man, mannose; GlcNAc, N-acetylglucosamine; Xyl, xylose; Fuc, fucose; Gal, galactose; NeuAc (neuraminic acid (sialic acid); *, reducing end of sugar chain that binds to asparagine.

FIGS. 21A and 21B show the nucleotide sequence (SEQ ID NO:11) and amino acid translation (SEQ ID NO:12) of the PLG polypeptide in BAP12-B2-150, which includes the alpha-amylase signal sequence (underlined; see SEQ ID NO:7 for the alpha-amylase signal coding sequence and SEQ ID NO:8 for the alpha-amylase signal amino acid sequence) fused to the mature human PLG polypeptide having the N289D substitution (see SEQ ID NO:5 for coding sequence and SEQ ID NO:6 for amino acid sequence). The GAC codon that results in the aspartic acid substitution at the position corresponding to Asn-289 of mature human PLG is shown in bold.

### DETAILED DESCRIPTION OF THE INVENTION

The present invention relates to the inventors' observation that recovery of recombinantly produced stable (i.e., inactive) plasminogen (PLG) from a plant host such as duckweed can be increased by eliminating N-linked glycosylation of the asparagine (Asn) residue occurring at the position corresponding to residue 289 of mature human PLG. In this manner, the present invention is drawn to methods and compositions for producing aglycosylated PLG polypeptides in plant-based expression systems. The methods comprise expressing an aglycosylated form of PLG in the plant host of interest, more particularly, a PLG polypeptide wherein the Asn residue that normally occurs at the position corresponding to residue 289 of mature human PLG is replaced with a residue that fails to support N-linked glycosylation at that site. Compositions of the invention include nucleic acid molecules encoding the aglycosylated PLG polypeptides of the invention, or fragment or variant thereof as defined elsewhere herein, expression constructs comprising these nucleic acid molecules, transformed plants comprising these expression constructs, and compositions comprising the isolated aglycosylated PLG polypeptides of the invention. The present invention also provides plasmin derived from the aglycosylated PLG polypeptides of the invention, for example, by activation of the PLG polypeptides with a suitable plasminogen activator, and compositions, including pharmaceutical compositions, comprising this plasmin.

The PLG to be expressed in the plant host of interest can be from any mammalian source, including, but not limited to, human, bovine, equine, porcine, ovine, canine, murine, and feline. Of particular interest to the present invention is human PLG, and variants of human PLG that retain the capability of being cleaved to a polypeptide having serine protease activity, for example, plasmin.

Human PLG is well known in the art, and its coding and amino acid sequences are available, as well as naturally occurring variants thereof. A representative human PLG-encoding nucleic acid sequence can be found at GenBank Accession No. M74220; the full-length human PLG-encoding sequence shown therein is set forth in SEQ ID NO:1. The full-length human PLG polypeptide (including its signal sequence at residues 1-19) encoded by SEQ ID NO:1 is set forth in SEQ ID NO:2 (see also GenBank Accession No. AAA36451). The coding sequence for the mature human PLG polypeptide (including the N-terminal glutamine residue) is set forth in SEQ ID NO:3; and the mature human PLG amino acid sequence encoded thereby is set forth in SEQ ID NO:4. See also the full-length human PLG polypeptide (including its signal sequence at residues 1-19) of GenBank Accession No. NP_{—}000292, set forth herein as SEQ ID NO:14, which has the sequence set forth in SEQ ID NO:2 with an isoleucine residue substituted for the valine residue at the position corresponding to position 701 of SEQ ID NO:2. This variant polypeptide is referred to herein as the V701I variant of full-length human PLG. A representative coding sequence for the V701I variant of full-length human PLG is set forth in SEQ ID NO:13 (see also GenBank Accession No. NM 000301). The corresponding mature polypeptide is set forth in SEQ ID NO:16, which has the sequence set forth in SEQ ID NO:4 with an isoleucine residue substituted for the valine residue at the position corresponding to position 682 of SEQ ID NO:4. This variant polypeptide is referred to herein as the V682I variant of mature human PLG. A representative coding sequence for the V682I variant of mature human PLG is set forth in SEQ ID:15. Also see the full-length human PLG polypeptide (including its signal sequence at residues 1-19) of GenBank Accession No. AAA60113, set forth herein as SEQ ID NO:18, which has the sequence set forth in SEQ ID NO:2 with an asparagine residue substituted for the aspartic acid residue at the position corresponding to position 472 of SEQ ID NO:2 and an isoleucine residue substituted for the valine residue at the position corresponding to position 701 of SEQ ID NO:2. This variant polypeptide is referred to herein as the D472N V701I variant of full-length human PLG. A representative coding sequence for the D472N V701I variant of full-length human PLG is set forth in SEQ ID NO:17 (see also GenBank Accession No. AH002941). The corresponding mature polypeptide is set forth in SEQ ID NO:20, which has the sequence set forth in SEQ ID NO:4 with an asparagine residue substituted for the aspartic acid residue at the position corresponding to position 453 of SEQ ID NO:4 and an isoleucine residue substituted for the valine residue at the position corresponding to position 682 of SEQ ID NO:4. This variant polypeptide is referred to herein as the D453N V682I variant of mature human PLG. A representative coding sequence for the D453N V682I variant of mature human PLG is set forth in SEQ ID:19.

Human PLG undergoes post-translational modification, more particularly glycosylation. When proteins such as human PLG move through the endoplasmic reticulum (ER) and Golgi subcellular compartments, sugar residue chains, or glycans, are attached, ultimately leading to the formation of a glycoprotein structure. The linkage between the sugar chains and the peptide occurs by formation of a chemical bond to only one of four protein amino acids: asparagine, serine, threonine, and hydroxylysine. Based on this linkage pattern, two basic types of sugar residue chains in glycoproteins have been recognized: the N-glycoside-linked sugar chain (also referred to as N-linked glycan or N-glycan), which binds to asparagine residues on the peptide; and the O-glycoside-linked sugar chain, which binds to serine, threonine, and hydroxylysine residues on the peptide.

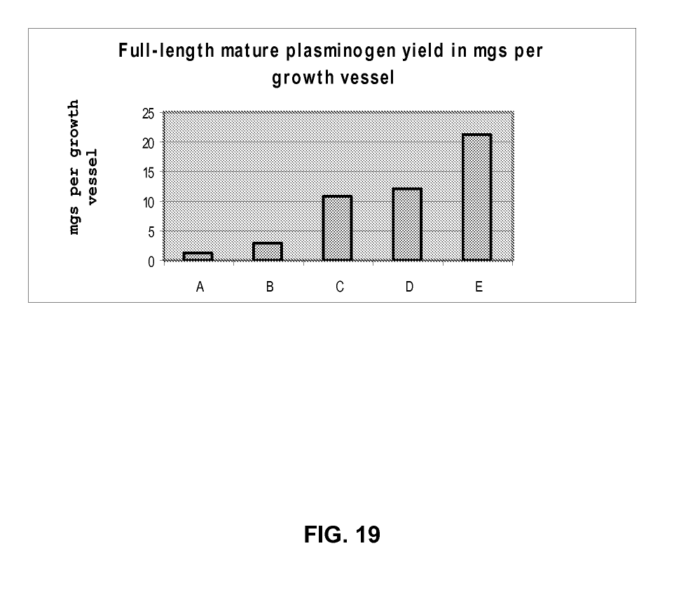

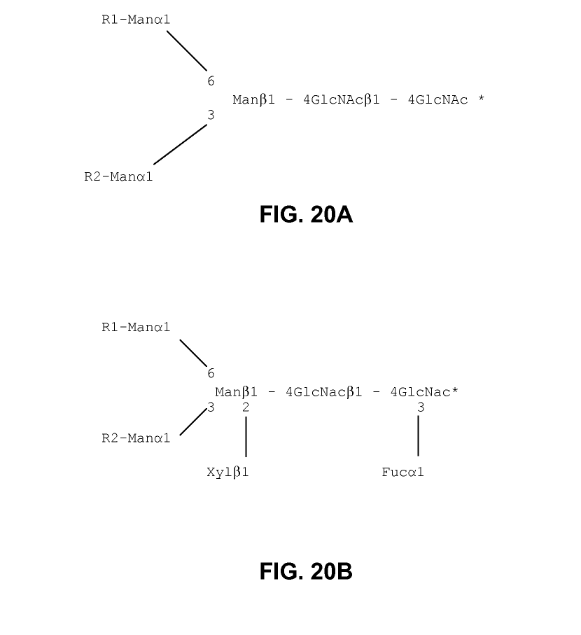

The N-glycoside-linked sugar chains, or N-glycans, have various structures (see, for example, Takahashi, ed. (1989) Biochemical Experimentation Method 23—Method for Studying Glycoprotein Sugar Chain (Gakujutsu Shuppan Center), but share a common oligomannosidic core (see FIG. 19A). The initial steps in the glycosylation pathway leading to the formation of N-glycans are conserved in plants and animals. However, the final steps involved in complex N-glycan formation differ (Lerouge et al. (1998) Plant Mol. Biol. 38:31-48; Steinkellner and Strasser (2003) Ann. Plant Rev.9:181-192). Plants produce glycoproteins with complex N-glycans having an oligomannosidic core bearing two N-acetylglucosamine (GlcNAc) residues that is similar to that observed in mammals. However, in plant glycoproteins this core is substituted by a β1,2-linked xylose residue (core xylose), which residue does not occur in humans, Lewis' epitopes, and an α1,3-linked fucose (core α[1,3]-fucose) instead of an α1,6-linked core fucose as in mammals (see, for example, Lerouge et al. (1998) Plant Mol. Biol. 38:31-48 for a review) (see also FIG. 20B).

The human PLG polypeptide comprises a single N-linked glycosylation site that resides at Asn-289 of the mature polypeptide (see, for example, the Asn residue at position 289 of SEQ ID NO:4, 16, or 20). By “N-linked glycosylation site” is intended an asparagine residue within the protein that permits the attachment of an N-linked glycan at that residue. As noted above, the human PLG polypeptide comprises two possible O-linked glycosylation sites, which occur at residues Ser-249 and Thr-346 of the mature PLG polypeptide (see, for example, the Ser and Thr residues at positions 249 and 346, respectively, of SEQ ID NO:4, 16, or 20). By “O-linked glycosylation site” is intended a serine, threonine, or hydroxylysine residue within the protein that permits the attachment of an O-linked glycan at that residue.

For purposes of the present invention, the terms “N-glycan,” “N-linked glycan,” and “glycan” are used interchangeably and refer to an N-linked oligosaccharide, e.g., one that is attached by an N-acetylglucosamine (GlcNAc) residue linked to the amide nitrogen of an asparagine residue in a protein. The predominant sugars found on glycoproteins are glucose, galactose, mannose, fucose, N-acetylgalactosamine (GalNAc), N-acetylglucosamine (GlcNAc), and sialic acid (e.g., N-acetyl-neuraminic acid (NeuAc)). The processing of the sugar groups occurs cotranslationally in the lumen of the ER and continues in the Golgi apparatus for N-linked glycoproteins.

By “oligomannosidic core structure” or “trimannose core structure” of a complex N-glycan is intended the core structure shown in FIG. 20A, wherein the core comprises three mannose (Man) and two N-acetylglucosamine (GlcNAc) monosaccharide residues that are attached to the asparagine residue of the glycoprotein. The asparagine residue is generally within the conserved peptide sequence Asn-Xxx-Thr or Asn-Xxx-Ser, where Xxx is any residue except proline, aspartate, or glutamate. Subsequent glycosylation steps yield the final complex N-glycan structure. The N-glycans attached to glycoproteins differ with respect to the number of branches (antennae) comprising peripheral sugars (e.g., GlcNAc, galactose, fucose, and sialic acid) that are added to the trimannose core structure. N-glycans are commonly classified according to their branched constituents (e.g., complex, high mannose, or hybrid).

Recombinantly produced human PLG when expressed in a plant-based expression system contains an N-linked glycan attached to Asn-289 of the mature human PLG sequence that has the typical plant N-glycan structure shown in FIG. 20B. Unlike PLG produced in mammalian host expression systems, recombinantly produced human PLG obtained from a plant-based expression system such as duckweed typically does not comprise O-linked glycans at the O-linked glycosylation sites within this polypeptide.

In contrast to the nucleic and amino acid sequences known in the art, the PLG sequences described herein are engineered so that the amino acid residue corresponding to the asparagine (Asn) residue at amino acid position 289 of mature human PLG, for example, the mature human PLG sequence set forth in SEQ ID NO:4, 6, or 20, cannot be glycosylated by attachment of an N-linked glycan. As such, the PLG polypeptides of the invention have been modified such that the amino acid at the position corresponding to Asn-289 of mature human PLG is an amino acid other than asparagine (Asn), as noted further herein below. Although the position of the amino acid substitution (i.e., residue 289) is described herein with reference to the mature human PLG sequence (see, for example, the sequence set forth in SEQ ID NO:4, 16, or 20), it is recognized that this position corresponds to residue 308 of the full-length human PLG polypeptide, which includes a 19-amino acid signal peptide at the N-terminal end of the mature PLG protein (see, for example, the full-length human PLG sequence set forth in SEQ ID NO:2, 14, or 18, wherein residues 1-19 constitute the signal peptide). Thus, while the present invention will refer to a modification of the codon encoding the residue corresponding to Asn-289 of mature human PLG, it is recognized that this modification occurs at the codon encoding the residue corresponding to Asn-308 of the full-length human PLG polypeptide.

By manipulating a PLG polypeptide such that post-translational attachment of N-linked glycans is prevented, it is possible to significantly increase yield of PLG from a plant-based expression system. The present invention thus provides methods and compositions for expressing high levels of stable (i.e., inactive) PLG. Thus, in one embodiment, the present invention provides a method for producing an aglycosylated PLG polypeptide, or biologically active fragment or variant thereof, in a plant of interest, such as duckweed, wherein the method comprises the steps of culturing a plant or plant cell, where the plant or plant cell is stably transformed with a nucleic acid molecule comprising a nucleotide sequence encoding an aglycosylated PLG described herein, or a fragment or variant thereof; and collecting the aglycosylated PLG polypeptide, or fragment or variant thereof, from the plant or plant cell. The nucleotide sequence encoding the aglycosylated PLG polypeptide, or fragment or variant thereof, may be operably linked to a nucleotide sequence encoding a signal peptide, as described herein below. Furthermore, the PLG-encoding nucleic acid molecule of the invention may be modified for enhanced expression in a plant, for example, by including plant-preferred codons in the coding sequence for the aglycosylated PLG polypeptide, or fragment or variant thereof, by the use of an operably linked sequence comprising a plant intron that is inserted upstream of the coding sequence, and/or the use of a leader sequence that increases the translation of the coding sequence for the aglycosylated PLG polypeptide. In some embodiments, two or more of these modifications are used in combination. Where the nucleotide sequence encoding the aglycosylated PLG polypeptide, or fragment or variant thereof, is operably linked to a nucleotide sequence encoding a signal peptide, the nucleotide sequence encoding the signal peptide may also comprise plant-preferred codons.

The methods of the invention may be used to express high levels of stable (i.e., inactive) PLG, or stable fragment or variant thereof, in a plant-based expression system. By expressing the PLG polypeptide or fragment or variant thereof in its aglycosylated form, it is possible to significantly increase the yield of stable PLG polypeptide or stable fragment or variant thereof relative to that obtained from the same plant-based expression system under comparable culture conditions when the PLG polypeptide or fragment or variant thereof is expressed in its glycosylated form. In this manner, in some embodiments of the invention, the yield of stable, aglycosylated PLG polypeptide or fragment or variant thereof, in mg per kilogram fresh weight of tissue, can be increased by at least about 1.5-fold, 2-fold, 2.5-fold, 3-fold, 3.5-fold, or at least about 4-fold over that obtainable from the same plant-based expression system that has been engineered to express the glycosylated form of the PLG polypeptide or fragment or variant thereof under comparable culture conditions. In other embodiments of the invention, the methods described herein provide for an increase in yield of stable, aglycosylated PLG polypeptide or fragment or variant thereof, in mg per kilogram fresh weight of tissue, that is at least about 4-fold, 4.5-fold, 5-fold, 5.5-fold, 6-fold, 6.5-fold, 7-fold, 7.5-fold, 8-fold, 8.5-fold, 9-fold, 9.5-fold, or at least about 10-fold greater than the yield obtainable from the same plant-based expression system that has been engineered to express the glycosylated form of the PLG polypeptide or fragment or variant thereof under comparable culture conditions.

Thus, in some embodiments, the plant host expressing the aglycosylated PLG polypeptide or fragment or variant thereof is a member of the duckweed family, for example, a species of Lemna, and the yield of stable, aglycosylated PLG polypeptide or fragment or variant thereof, in mg per kilogram fresh weight of tissue, is at least about 1.5-fold, 2-fold, 3-fold, 3.5-fold, 4-fold, 4.5-fold, 5-fold, 5.5-fold, 6-fold, 6.5-fold, 7-fold, 7.5-fold, 8-fold, 8.5-fold, 9-fold, 9.5-fold, or at least about 10-fold greater than the yield obtainable from the same duckweed family member that has been engineered to express the glycosylated form of the PLG polypeptide or fragment or variant thereof under comparable culture conditions.

The invention also encompasses plants, and plant parts thereof, including plant cells, transformed with expression cassettes capable of expressing an aglycosylated PLG polypeptide, or fragment or variant thereof, in a plant, such as duckweed. Also provided are nucleic acid molecules comprising a nucleotide sequence encoding the aglycosylated PLG polypeptides of the invention, or fragments or variants thereof, including PLG-encoding sequences comprising plant-preferred codons.

Unless defined otherwise, all technical and scientific terms used herein have the same meaning as commonly understood by one of ordinary skill in the art to which the invention pertains. Although any methods and materials similar to or equivalent to those described herein can be used in the practice or testing of the present invention, exemplary methods and materials are described herein.

Recombinant Nucleic Acid Molecules and Aglycosylated PLG Polypeptides

The present invention is directed to novel nucleic acid molecules that allow for improved production of plasminogen (PLG) in plant-based expression systems, for example, in a duckweed expression system. The nucleic acid molecules of the invention comprise a nucleotide sequence encoding a plasminogen (PLG) polypeptide, wherein the codon for the asparagine (Asn) residue at the position corresponding to Asn-289 of mature human PLG, or fragment or variant thereof as defined herein below, has been modified such that the encoded residue is not Asn. Such a modification results in the substitution of a residue at that position, where the substituted residue is one that will not support N-linked glycosylation at that site within the PLG polypeptide. In this manner, expression of the nucleic acid molecules of the invention in a plant-based expression system yields an aglycosylated PLG polypeptide. Preferably the substitution at the position corresponding to Asn-289 of mature human PLG is conservative in nature so that the aglycosylated PLG polypeptide retains the ability to be activated to a polypeptide having serine protease activity, such as plasmin. Expression constructs comprising these PLG-encoding nucleic acid molecules, transgenic plants comprising these expression constructs, the encoded aglycosylated PLG polypeptides and plasmin derived therefrom, compositions comprising these PLG polypeptides, and compositions comprising the plasmin derived therefrom are also provided.

As used herein, a “nucleic acid sequence” or “nucleic acid sequences” means a DNA or RNA sequence. The term encompasses sequences that include any of the known base analogues of DNA and RNA including, but not limited to, adenine, cytosine, thymine, guanine, uracil, 4-acetylcytosine, 8-hydroxy-N-6-methyladenosine, aziridinylcytosine, pseudoisocytosine, 5-(carboxyhydroxylmethyl)uracil, 5-fluorouracil, 5-bromouracil, 5-carboxymethylaminomethyl-2-thiouracil, 5-carboxymethylaminomethyluracil, dihydrouracil, inosine, N6-isopentenyladenine, 1-methyladenine, 1-methylpseudouracil, 1-methylguanine, 1-methylinosine, 2,2-dimethylguanine, 2-methyladenine, 2-methylguanine, 3-methylcytosine, 5-methylcytosine, N6-methyladenine, 7-methylguanine, 5-methylaminomethyluracil, 5-methoxyaminomethyl-2-thiouracil, beta-D-mannosylqueosine, 5′-methoxycarbonylmethyluracil, 5-methoxyuracil, 2-methylthio-N-6-isopentenyladenine, uracil-5-oxyacetic acid methylester, uracil-5-oxyacetic acid, oxybutoxosine, pseudouracil, queosine, 2-thiocytosine, 5-methyl-2-thiouracil, 2-thiouracil, 4-thiouracil, 5-methyluracil, -uracil-5-oxyacetic acid methylester, uracil-5-oxyacetic acid, pseudouracil, queosine, 2-thiocytosine and 2,6-diaminopurine.

As used herein, the term “nucleic acid molecule” is intended to include DNA molecules (e.g., cDNA or genomic DNA) and RNA molecules (e.g., mRNA) and analogs of the DNA or RNA generated using nucleotide analogs. The nucleic acid molecule can be single-stranded or double-stranded, but preferably is double-stranded DNA.

As used herein, a “coding sequence” means a nucleic acid sequence that encodes a particular amino acid molecule (i.e., polypeptide), and is a nucleic acid sequence that is transcribed (in the case of DNA) and translated (in the case of mRNA) into a polypeptide in vitro or in vivo when placed under the control of appropriate regulatory sequences. The boundaries of the coding sequence are determined by a start codon at a 5′ (amino) terminus and a translation stop codon at a 3′ (carboxy)terminus. A coding sequence can include, but is not limited to, viral nucleic acid sequences, cDNA from prokaryotic or eukaryotic mRNA, genomic DNA sequences from prokaryotic or eukaryotic DNA and even synthetic DNA sequences.

For purposes of the present invention, a “PLG-encoding sequence” is intended to mean a coding sequence for an aglycosylated PLG polypeptide of the invention, and can encode a fragment or variant of an aglycosylated PLG polypeptide of the invention.

The invention encompasses isolated or substantially purified nucleic acid molecules and polypeptide compositions. An “isolated” or “purified” nucleic acid molecule or polypeptide, or biologically active fragment or variant thereof, is substantially or essentially free from components that normally accompany or interact with the nucleic acid molecule or its encoded protein as found in its naturally occurring environment. Thus, an isolated or purified nucleic acid molecule or polypeptide, or biologically active fragment or variant thereof, is substantially free of other cellular material or culture medium when produced by recombinant techniques, or substantially free of chemical precursors or other chemicals when chemically synthesized. Optimally, an “isolated” nucleic acid molecule is free of sequences (optimally protein encoding sequences) that naturally flank the polynucleotide (i.e., sequences located at the 5′ and 3′ ends of the nucleic acid molecule) in the genomic DNA of an organism from which the nucleic acid molecule is obtained. For example, the isolated nucleic acid molecule can contain less than about 5 kb, 4 kb, 3 kb, 2 kb, 1 kb, 0.5 kb or 0.1 kb of the nucleic acid sequences that naturally flank the nucleic acid molecule in genomic DNA of the cell from which it is obtained. An “isolated” aglycosylated PLG polypeptide, or biologically active fragment or variant thereof, that is substantially free of cellular material includes preparations of PLG protein having less than about 30%, 20%, 10%, 5%, or 1% (by dry weight) of contaminating protein. When the aglycosylated PLG polypeptide of the invention or biologically active fragment or variant thereof is recombinantly produced, optimally culture medium represents less than about 30%, 20%, 10%, 5%, or 1% (by dry weight) of chemical precursors or non-protein-of-interest chemicals.

In some embodiments of the present invention, the aglycosylated PLG polypeptides of the invention, or biologically active fragments or variants thereof, are isolated from the plant host material. In other embodiments, the aglycosylated PLG polypeptides of the invention, or biologically active fragments or variants thereof, are secreted into the plant culture medium, and then isolated from the plant culture medium. In yet other embodiments, the aglycosylated PLG polypeptides of the invention, or biologically active fragments or variants thereof, are isolated from the plant host material and the plant culture medium. The aglycosylated PLG polypeptides of the invention, or biologically active fragments or variants thereof, can be harvested from the plant host material and/or the plant culture medium and purified using any conventional means known in the art, including, but not limited to, chromatography, electrophoresis, dialysis, solvent-solvent extraction, and the like.

In some embodiments, these purified aglycosylated PLG polypeptides, or biologically active fragments or variants thereof, obtained from the plant host can include at least about 0.001%, 0.005%, 0.1%, 0.5%, 1%, 1.5%, 2%, 2.5%, 3%, 3.5%, 4%, 4.5%, 5%, 5.5%, 6%, 6.5%, 7%, 7.5%, 8%, 8.5%, 9%, 9.5%, 10%, 15%, 20%, 25%, or up to about 30% (by dry weight) of contaminating plant protein. In other embodiments, these purified aglycosylated PLG polypeptides, or biologically active fragments or variants thereof, obtained from the plant host can include at least 0.001% up to about 25%, at least 0.001% up to about 20%, at least 0.001% up to about 15%, at least 0.001% up to about 10%, at least 0.001% up to about 9.5%, at least 0.001% up to about 9.0%, at least 0.001% up to about 8.5%, at least 0.001% up to about 8%, at least 0.001% up to about 7.5%, at least 0.001% up to about 6.5%, at least 0.001% up to about 6%, at least 0.001% up to about 5.5%, at least 0.001% up to about 5%, at least 0.001% up to about 4.5%, at least 0.001% up to about 4%, at least 0.001% up to about 3.5%, at least 0.001% up to about 3%, at least 0.001% up to about 2.5%, at least 0.001% up to about 2%, at least 0.001% up to about 1.5%, at least 0.001% up to about 1%, at least 0.001% up to about 0.5%, at least 0.001% up to about 0.1%, or at least 0.001% up to about 0.005% (by dry weight) of contaminating plant protein.

In some embodiments, where the aglycosylated PLG polypeptide, or biologically active fragment or variant thereof, is collected from the plant culture medium, the plant culture medium in the purified aglycosylated PLG polypeptide preparation, or in the preparation of purified biologically active fragment or variant thereof, can represent at least about 0.001%, 0.005%, 0.1%, 0.5%, 1%, 1.5%, 2%, 2.5%, 3%, 3.5%, 4%, 4.5%, 5%, 5.5%, 6%, 6.5%, 7%, 7.5%, 8%, 8.5%, 9%, 9.5%, 10%, 15%, 20%, 25%, or up to about 30% (by dry weight) of chemical precursors or non-protein-of-interest chemicals within the purified aglycosylated PLG polypeptide preparation or within the preparation of purified biologically active variant of fragment thereof. In other embodiments, where the aglycosylated PLG polypeptide, or biologically active fragment or variant thereof, is collected from the plant culture medium, the plant culture medium in the purified aglycosylated PLG polypeptide preparation, or in the preparation of purified biologically active fragment or variant thereof, can represent at least 0.001% up to about 25%, at least 0.001% up to about 20%, at least 0.001% up to about 15%, at least 0.001% up to about 10%, at least 0.001% up to about 9.5%, at least 0.001% up to about 9.0%, at least 0.001% up to about 8.5%, at least 0.001% up to about 8%, at least 0.001% up to about 7.5%, at least 0.001% up to about 6.5%, at least 0.001% up to about 6%, at least 0.001% up to about 5.5%, at least 0.001% up to about 5%, at least 0.001% up to about 4.5%, at least 0.001% up to about 4%, at least 0.001% up to about 3.5%, at least 0.001% up to about 3%, at least 0.001% up to about 2.5%, at least 0.001% up to about 2%, at least 0.001% up to about 1.5%, at least 0.001% up to about 1%, at least 0.001% up to about 0.5%, at least 0.001% up to about 0.1%, or at least 0.001% up to about 0.005% (by dry weight) of chemical precursors or non-protein-of-interest chemicals within the purified aglycosylated PLG polypeptide preparation or within the preparation of purified biologically active fragment or variant thereof.

In some embodiments, isolation and purification from the plant host, and where secreted, from the culture medium, results in recovery of purified aglycosylated PLG polypeptide, or purified biologically active fragment or variant thereof, that is free of contaminating plant protein, free of plant culture medium components, and/or free of both contaminating plant protein and plant culture medium components.

Although the coding sequence for any mammalian source of plasminogen can serve as the reference sequence that is to be modified to obtain an aglycosylated PLG polypeptide of the invention, preferably the PLG coding sequence is a sequence encoding the full-length human PLG sequence set forth in SEQ ID NO:2, a sequence encoding the mature human PLG sequence set forth in SEQ ID NO:4, or a sequence encoding a fragment or variant of the full-length human PLG sequence or a fragment or variant of the mature human PLG sequence, as defined elsewhere herein. In other embodiments, the PLG coding sequence is a sequence encoding the full-length human PLG sequence set forth in SEQ ID NO:14 or 18, a sequence encoding the mature human PLG sequence set forth in SEQ ID NO:16 or 20, or a sequence encoding a fragment or variant of the full-length human PLG sequence or a fragment or variant of the mature human PLG sequence, as defined elsewhere herein. A representative nucleic acid sequence encoding the full-length human PLG of SEQ ID NO:2 is set forth in SEQ ID NO:1, and a representative nucleic acid sequence encoding the mature human PLG of SEQ ID NO:4 is set forth in SEQ ID NO:3. A representative nucleic acid sequence encoding the full-length human PLG of SEQ ID NO:14 is set forth in SEQ ID NO:13, and a representative nucleic acid sequence encoding the mature human PLG of SEQ ID NO:16 is set forth in SEQ ID NO:15. A representative nucleic acid sequence encoding the full-length human PLG of SEQ ID NO:18 is set forth in SEQ ID NO:17, and a representative nucleic acid sequence encoding the mature human PLG of SEQ ID NO:20 is set forth in SEQ ID NO:19. One of ordinary skill in the art can use any method to modify a sequence encoding the full-length human PLG set forth in SEQ ID NO:2, 14, or 18 (for example, the coding sequence set forth in SEQ ID NO:1, SEQ ID NO:13, or SEQ ID NO:17, respectively), or a sequence encoding the mature human PLG set forth in SEQ ID NO:4, 16, or 20 (for example, the coding sequence set forth in SEQ ID NO:3, SEQ ID NO:15, or SEQ ID NO:19, respectively), SEQ ID NO:16 (for example, the coding sequence set forth in SEQ ID NO:15) or to modify a sequence encoding a fragment or variant of the full-length human PLG of SEQ ID NO:2, 14, or 18, or of the mature human PLG of SEQ ID NO:2, 16, or 20, respectively, so that it encodes an aglycosylated PLG polypeptide, or aglycosylated fragment or variant thereof, that is capable of being activated to a polypeptide having serine protease activity, for example, activated to plasmin.

Any acceptable method known in the art can be used to modify the reference PLG-encoding sequence, for example, a sequence encoding the full-length PLG polypeptide set forth in SEQ ID NO:2, 14, or 18, or the mature PLG polypeptide set forth in SEQ ID NO:4, 16, or 20, to obtain a sequence encoding an aglycosylated PLG polypeptide of the invention. Methods for mutagenesis and nucleotide sequence alterations are well known in the art. See, for example, Kunkel (1985) Proc. Natl. Acad. Sci. USA 82:488-492; Kunkel et al. (1987) Methods in Enzymol. 154:367-382; U.S. Pat. No. 4,873,192 (herein incorporated by reference); Walker and Gaastra, eds. (1983) Techniques in Molecular Biology (MacMillan Publishing Company, NY) and the references cited therein. Furthermore, the PLG-encoding sequences of the invention may also be chemically synthesized and assembled by any of a number of techniques prior to expression in the plant host of interest. See, for example, U.S. Pat. No. 4,500,707; Balland et al. (1985) Biochimie 67:725-736; and Edge et al. (1982) Nature 292:756-762.

As used herein, “aglycosylated PLG polypeptide” means a PLG polypeptide lacking at least N-linked glycosylation at the amino acid residue corresponding to the asparagine (Asn) residue at position 289 of the mature human PLG sequence (i.e., Asn-289 of SEQ ID NO:4, 16, or 20). In this manner, the PLG polypeptide, for example, human PLG or fragment or variant thereof as defined herein below, when expressed in a plant-based expression system, does not contain an N-linked glycan attached at the amino acid residue corresponding to Asn-289 of the mature human PLG sequence.

By “amino acid residue corresponding to Asn-289 of the mature human PLG sequence” is intended the amino acid residue within the aglycosylated PLG polypeptide of the invention that appears opposite Asn-289 of the mature human PLG sequence when the PLG polypeptide of the invention is aligned with the mature human PLG sequence set forth in SEQ ID NO:4, 16, or 20 for maximum homology using the alignment program identified herein below. Thus, while the encoded aglycosylated PLG polypeptide of the invention can be human PLG, with a substitution of Asn-289 of the mature human PLG sequence of SEQ ID NO:4, 16, or 20 such that the residue at this position is not Asn, the encoded aglycosylated PLG polypeptide of the invention also can be a variant of human PLG, wherein the amino acid residue that corresponds to Asn-289 of mature human PLG has been substituted such that it is not an Asn.

As noted above, recombinantly produced PLG, for example, human PLG, obtained from a plant-based expression system such as duckweed typically does not comprise O-linked glycans at the O-linked glycosylation sites within this polypeptide, which occur at positions corresponding to Ser-249 and Thr-346 of the mature human PLG sequence set forth in SEQ ID NO:4, 16, or 20. Thus, unlike aglycosylated PLG produced in a mammalian host cell, the aglycosylated PLG polypeptides of the present invention not only lack an N-linked glycan at the residue position corresponding to Asn-289 of the mature human PLG sequence, but also may retain the O-linked glycosylation sites occurring at the amino acid residues corresponding to Ser-249 and Thr-346, respectively, of the mature human PLG sequence. By “amino acid residue corresponding to Ser-249 of the mature human PLG sequence” is intended the amino acid residue within the aglycosylated PLG polypeptide of the invention that appears opposite Ser-249 of the mature human PLG sequence when the PLG polypeptide of the invention is aligned with the mature human PLG sequence set forth in SEQ ID NO:4, 16, or 20 for maximum homology using the alignment program identified herein below. By “amino acid residue corresponding to Thr-346 of the mature human PLG sequence” is intended the amino acid residue within the aglycosylated PLG polypeptide of the invention that appears opposite Thr-346 of the mature human PLG sequence when the PLG polypeptide of the invention is aligned with the mature human PLG sequence set forth in SEQ ID NO:4, 16, or 20 for maximum homology using the alignment program identified herein below. In some embodiments, the aglycosylated PLG polypeptides of the present invention can be obtained by modification of the codon for the Asn residue corresponding to Asn-289 of the mature human PLG sequence, while still retaining the codon for the native Ser and/or Thr residues corresponding to Ser-249 and Thr-346, respectively, of mature human PLG. For example, when the aglycosylated PLG polypeptides of the invention are produced in duckweed as the host expression system, these aglycosylated PLG polypeptides lack an N-linked glycan at the residue position corresponding to Asn-289 of the mature human PLG sequence and also lack the O-linked glycans at the native O-linked glycosylation sites occurring at the amino acid residues corresponding to Ser-249 and Thr-346, respectively, of the mature human PLG sequence.

It is recognized that in those occasional instances where O-linked glycosylation is detected within the plant host of interest, the coding sequence for the PLG polypeptide of interest (for example, full-length human PLG of SEQ ID NO:2, 14, or 18, or mature human PLG of SEQ ID NO:4, 16, or 20) can be modified at the codons for the Ser and/or Thr residues corresponding to Ser-249 and Thr-346, respectively, of mature human PLG of SEQ ID NO:4, 16, or 20 such that the residues at the respective positions are not Ser and/or Thr (or hydroxylysine), to prevent O-linked glycosylation at these sites.

Methods for determining the presence or absence of N-linked glycans at N-linked glycosylation sites and O-linked glycans at O-linked glycosylation sites within a polypeptide, in this case, a PLG polypeptide, are well known in the art. Thus, for example, a PLG polypeptide can be analyzed for its glycosylation profile. By “glycosylation profile” is intended the characteristic “fingerprint” of the representative N-glycan or O-glycan species that have been released from the PLG polypeptide, either enzymatically or chemically, and then analyzed for their carbohydrate structure, for example, using LC-HPLC, or MALDI-TOF MS, and the like. See, for example, the review in Current Analytical Chemistry, Vol. 1, No. 1 (2005), pp. 28-57; see also, Royle et al. (2002) Analytical Biochem. 304:70-90; Merry et al. (2002) Analytical Biochem. 304:91-99; Pouria et al. (2004) Analytical Biochem. 330:257-263; the contents of each of which are herein incorporated by reference in their entirety.

For example, any nucleotide sequence encoding the full-length human PLG polypeptide set forth in SEQ ID NO:2, 14, or 18 (for example, the coding sequence set forth in SEQ ID NO:1, 13, or 17, respectively), or the mature human PLG polypeptide set forth in SEQ ID NO:4, 16, or 20 (for example, the coding sequence set forth in SEQ ID NO:3, 15, or 19, respectively) can be modified to encode a mature PLG polypeptide comprising the sequence set forth in SEQ ID NO:4, 16, or 20 in which the amino acid residue at position 289 is an amino acid other than Asn. Although one or both of the O-linked glycosylation sites at the residue positions corresponding to Ser-249 and Thr-346 of the mature human PLG sequence of SEQ ID NO:4, 16, or 20 also can be modified, the aglycosylated PLG polypeptide of the present invention typically retains at least the O-linked glycosylation site at the residue position corresponding to Thr-346 of the mature human PLG sequence of SEQ ID NO:4, 16, or 20 and may retain both of the O-linked glycosylation sites at the residue positions corresponding to Ser-249 and Thr-346 of the mature human PLG sequence of SEQ ID NO:4, 16, or 20.

Thus, in some embodiments, the present invention provides an isolated nucleic acid molecule comprising a nucleotide sequence encoding a mature PLG polypeptide that differs from the mature human PLG polypeptide set forth in SEQ ID NO:4, 16, or 20 only by having an amino acid substitution for the asparagine (Asn) residue at position 289 of SEQ ID NO:4, 16, or 20 such that the residue at this position is any other residue except Asn. In such embodiments, the nucleotide sequence encoding this mature PLG polypeptide comprises a codon for the substituted residue at position 289 of SEQ ID NO:4, 16, or 20 rather than a codon for Asn. Although any residue other than Asn can be substituted, of particular interest are conservative substitutions so that the aglycosylated PLG polypeptide, when expressed, is capable of being activated to a polypeptide having serine protease activity, for example, activated to plasmin.

One of ordinary skill in the art understands that amino acids within the same conservative group can typically substitute for one another without substantially affecting a function of a protein. Such conservative groups are set forth in Table 1 and are based on shared properties.

TABLE 1
Amino Acid Conservative Substitutions.

| | Side | | | Common |
|---|---|---|---|---|
| | Chain | | Hydropathy | Conservative |
| Residue | Polarity | Side Chain pH | Index | Substitution |
| Ala (A) | Non-polar | Neutral | 1.8 | Val, Leu, Ile |
| Arg (R) | Polar | Basic | −4.5 | Lys, Gln, Asn |
| | | (strongly) | | |
| Asn (N) | Polar | Neutral | −3.5 | Gln, His, Lys, |
| | | | | Arg, Asp |
| Asp (D) | Polar | Acidic | −3.5 | Glu |
| Cys (C) | Non-polar | Neutral | 2.5 | Ser |
| Gln (Q) | Polar | Neutral | −3.5 | Asn |
| Glu (E) | Polar | Acidic | −3.5 | Asp |
| Gly (G) | Non-polar | Neutral | −0.4 | Pro |
| His (H) | Polar | Basic (weakly) | −3.2 | Asn, Gln, |
| | | | | Lys, Arg |
| Ile (I) | Non-polar | Neutral | 4.5 | Leu, Val, Met, |
| | | | | Ala, Phe |
| Leu (L) | Non-polar | Neutral | 3.8 | Ile, Val, Met, |
| | | | | Ala, Phe |
| Lys (K) | Polar | Basic | −3.9 | Arg, Gln, Asn |
| Met (M) | Non-polar | Neutral | 1.9 | Leu, Phe, Ile |
| Phe (F) | Non-polar | Neutral | 2.8 | Leu, Val, Ile, Ala |
| Pro (P) | Non-polar | Neutral | −1.6 | Gly |
| Ser (S) | Polar | Neutral | −0.8 | Thr |
| Thr (T) | Polar | Neutral | −0.7 | Ser |
| Trp (W) | Non-polar | Neutral | −0.9 | Tyr, Phe |
| Tyr (Y) | Polar | Neutral | −1.3 | Trp, Phe, Thr, Ser |
| Val (V) | Non-polar | Neutral | 4.2 | Ile, Leu, Met, |
| | | | | Phe, Ala |

As such, one of ordinary skill in the art readily can determine those residues that represent conservative substitutions for Asn, and which can thus be used when designing the aglycosylated PLG polypeptides of the invention.

Thus, in some embodiments, the invention provides isolated nucleic acid molecules comprising a nucleotide sequence encoding a mature PLG polypeptide that differs from the mature human PLG polypeptide set forth in SEQ ID NO:4, 16, or 20 only by having a glutamine (Gln or Q), histidine (His or H), lysine (Lys or K), arginine (Arg or R), or aspartic acid (Asp or D) residue substituted for the Asn residue at position 289 of SEQ ID NO:4, 16, or 20. Such substitutions are referred to herein with the following nomenclature: N289Q, N289H, N289K, N289R, and N289D, for the Gln, His, Lys, Arg, and Asp substitutions, respectively. In one such embodiment, the PLG-encoding nucleic acid molecule of the invention comprises a coding sequence for the mature PLG polypeptide set forth in SEQ ID NO:6, wherein the encoded mature PLG polypeptide differs from the mature human PLG of SEQ ID NO:4 in having the N289D substitution.

As noted above, where O-linked glycosylation is detected in the expressed PLG polypeptide, the nucleotide sequence encoding the mature PLG polypeptide that comprises the sequence set forth in SEQ ID NO:4, 16, or 20 with the N289Q, N289H, N289K, N289R, or N289D substitution can be further modified such that the codon for the residue corresponding to Ser-249 and/or Thr-346 can be altered to encode a residue that does not support O-linked glycosylation at that site (i.e., a residue that is not Ser, Thr, or hydroxylysine).

The nucleic acid molecules of the invention can comprise any nucleotide sequence that encodes the desired aglycosylated PLG polypeptide of the invention. In this manner, where the encoded aglycosylated PLG polypeptide of the invention is, for example, a a mature PLG polypeptide having the amino acid sequence of the mature human PLG of SEQ ID NO:4 with a substitution selected from the group consisting of N289Q, N289H, N289K, N289R, and N289D, the nucleotide sequence encoding that mature PLG polypeptide can be, for example, the sequence set forth in SEQ ID NO:1 or SEQ ID NO:3, but with an appropriate codon substitution appearing at nucleotides 922-924 of SEQ ID NO:1 or at nucleotides 865-867 of SEQ ID NO:3 such that the encoded residue at position 289 of the mature PLG polypeptide is Gln, His, Lys, Arg, or Asp instead of Asn. In like manner, where the encoded aglycosylated PLG polypeptide of the invention is, for example, a mature PLG polypeptide having the amino acid sequence of the mature human PLG of SEQ ID NO:16 with a substitution selected from the group consisting of N289Q, N289H, N289K, N289R, and N289D, the nucleotide sequence encoding that mature PLG polypeptide can be, for example, the sequence set forth in SEQ ID NO:13 or SEQ ID NO:15, but with an appropriate codon substitution appearing at nucleotides 922-924 of SEQ ID NO:13 or at nucleotides 865-867 of SEQ ID NO:15 such that the encoded residue at position 289 of the mature PLG polypeptide is Gln, His, Lys, Arg, or Asp instead of Asn. Similarly, where the encoded aglycosylated PLG polypeptide of the invention is, for example, a mature PLG polypeptide having the amino acid sequence of the mature human PLG of SEQ ID NO:20 with a substitution selected from the group consisting of N289Q, N289H, N289K, N289R, and N289D, the nucleotide sequence encoding that mature PLG polypeptide can be, for example, the sequence set forth in SEQ ID NO:17 or SEQ ID NO:19, but with an appropriate codon substitution appearing at nucleotides 922-924 of SEQ ID NO:17 or at nucleotides 865-867 of SEQ ID NO:19 such that the encoded residue at position 289 of the mature PLG polypeptide is Gln, His, Lys, Arg, or Asp instead of Asn. Codons for the naturally occurring amino acids are well known in the art, including those codons that are most frequently used in particular host organisms used to express recombinant proteins.

Thus, for example, where the aglycosylated PLG polypeptide of the invention is, for example, a mature PLG polypeptide having the amino acid sequence of the mature human PLG of SEQ ID NO:4, 16, or 20 with the N289Q substitution, the nucleic acid molecule of the invention can comprise any nucleotide sequence encoding this mature PLG polypeptide, wherein the sequence encoding the N289Q substitution can be selected from the two universal triplet codons for glutamine, i.e., CAA and CAG. Similarly, where the aglycosylated PLG polypeptide of the invention is, for example, a mature PLG polypeptide having the amino acid sequence of the mature human PLG of SEQ ID NO:4, 16, or 20 with the N289H substitution, the nucleic acid molecule of the invention can comprise any nucleotide sequence encoding this mature PLG polypeptide, wherein the sequence encoding the N289H substitution can be selected from the two universal triplet codons for histidine, i.e., CAT and CAC.

In like manner, where the aglycosylated PLG polypeptide of the invention is, for example, a mature PLG polypeptide having the amino acid sequence of the mature human PLG of SEQ ID NO:4, 16, or 20 with the N289K substitution, the nucleic acid molecule of the invention can comprise any nucleotide sequence encoding this mature PLG polypeptide, wherein the sequence encoding the N289K substitution can be selected from the two universal triplet codons for lysine, i.e., AAA and AAG. Similarly, where the aglycosylated PLG polypeptide of the invention is, for example, a mature PLG polypeptide having the amino acid sequence of the mature human PLG of SEQ ID NO:4, 16, or 20 with the N289R substitution, the nucleic acid molecule of the invention can comprise any nucleotide sequence encoding this mature PLG polypeptide, wherein the sequence encoding the N289R substitution can be selected from the four universal triplet codons for arginine, i.e., CGT, CGC, CGA, and CGG.

In like manner, where the aglycosylated PLG polypeptide of the invention is, for example, a mature PLG polypeptide having the amino acid sequence of the mature human PLG of SEQ ID NO:4, 16, or 20 with the N289D substitution, the nucleic acid molecule of the invention can comprise any nucleotide sequence encoding this mature PLG polypeptide, wherein the sequence encoding the N289D substitution can be selected from the two universal triplet codons for aspartic acid, i.e., GAC and GAT.

Thus, the present invention provides isolated nucleic acid molecules comprising a nucleotide sequence encoding an aglycosylated PLG polypeptide lacking at least N-linked glycosylation at the amino acid residue corresponding to the asparagine (Asn) residue at position 289 of the mature human PLG sequence (i.e., Asn-289 of SEQ ID NO:4, 16, or 20), as well as the isolated aglycosylated PLG polypeptides encoded thereby. Preferably the encoded and isolated aglycosylated PLG polypeptides of the invention also retain at least the O-linked glycosylation site at the amino acid residue corresponding to the threonine (Thr) residue at position 346 of the mature human PLG sequence (i.e., Thr-346 of SEQ ID NO:4, 16, or 20), or both the O-linked glycosylation site at the amino acid residue corresponding to the serine (Ser) residue at position 249 of the mature human PLG sequence (i.e., Ser-249 of SEQ ID NO:4, 16, or 20) and the O-linked glycosylation site at the amino acid residue corresponding to Thr-346 of the mature human PLG sequence. In some embodiments, the aglycosylated PLG polypeptides of the invention comprise the sequence for mature human PLG as set forth in SEQ ID NO:4, 16, or 20 with an amino acid substitution for Asn-289 such that the substituted residue is one that does not allow for attachment of an N-linked glycan at that site within the PLG polypeptide. Exemplary substitutions include those that are conservative, and thus the aglycosylated PLG polypeptide of the invention comprises the sequence set forth in SEQ ID NO:4, 16, or 20 with a substitution selected from the group consisting of N289Q, N289H, N289K, N289R, and N289D. One such aglycosylated PLG polypeptide comprises the sequence set forth in SEQ ID NO:6, which differs from the mature human PLG of SEQ ID NO:4 in having the N289D substitution. When expressed in a plant-based expression system, such as duckweed, such PLG polypeptides are devoid of N-linked and O-linked glycans.

### Fragments and Variants of PLG-Encoding Polynucleotides and Aglycosylated PLG Polypeptides

Although the foregoing description relates to nucleic acid molecules comprising a nucleotide sequence encoding aglycosylated PLG polypeptides comprising the sequence of mature human PLG with a substitution of a residue other than Asn for Asn-289 of mature human PLG (for example, SEQ ID NO:4, 16, or 20 with a substitution selected from the group consisting of N289Q, N289H, N289K, N289R, and N289D), and the aglycosylated PLG polypeptides encoded thereby, it is recognized that the present invention contemplates nucleic acid molecules comprising a nucleotide sequence encoding fragments and variants of these aglycosylated PLG polypeptides, as well as the isolated aglycosylated PLG fragments and aglycosylated variant PLG polypeptides encoded thereby.

In this manner, one of ordinary skill in the art understands that modifications to nucleotide or amino acid sequences include substitutions, insertions, and deletions. These modifications can be introduced into the PLG-encoding sequences or PLG amino acid sequences described herein without abolishing structure and, ultimately, function. PLG-encoding sequences and PLG amino acid sequences containing such modifications can be used in the methods described herein.

Thus, the present invention contemplates biologically active fragments and variants of the aglycosylated PLG polypeptides disclosed herein, so long as the PLG fragment or variant PLG polypeptide retains the substitution at the amino acid residue corresponding to Asn-289 of mature human PLG, thereby preventing attachment of an N-linked glycan at that site within the PLG fragment or variant PLG polypeptide. In this manner, the PLG fragments and variant PLG polypeptides are also aglycosylated when produced in the plant host of interest. Thus, the PLG fragment and variant PLG polypeptides preferably also retain at least the O-linked glycosylation site at the amino acid residue corresponding to Thr-346 of mature human PLG, and can retain both the O-linked glycosylation site at the amino acid residue corresponding to Ser-249 and the O-linked glycosylation site at the amino acid residue corresponding to Thr-346 of mature human PLG. By “biologically active” is intended the PLG fragment or variant PLG polypeptide is capable of being activated to produce a polypeptide having the protease activity of the plasmin family of proteases (Enzyme Class 3.4.21.7). Methods for determining such protease activity are well known in the art and include the assays described in the Experimental section herein below.

A biologically active variant of an aglycosylated PLG polypeptide disclosed herein can be derived from deletion (so-called truncation) or addition of one or more amino acids to the N-terminal and/or C-terminal end of the aglycosylated PLG polypeptide; deletion or addition of one or more amino acids at one or more sites in the aglycosylated PLG polypeptide; or substitution of one or more amino acids at one or more sites in the aglycosylated PLG polypeptide. Such biologically active variants may result from, e.g., genetic polymorphism or from human manipulation (i.e., recombinantly or chemically produced PLG-encoding nucleic or amino acid sequences). Biologically active variants of a reference aglycosylated PLG polypeptide will have at least about 50%, 60%, 65%, 70%, more typically at least about 75%, 80%, 85%, preferably at least about 90%, 91%, 92%, 93%, 94%, 95%, 96%, 97%, 98%, 99% or more sequence identity to the amino acid sequence of the reference aglycosylated PLG polypeptide. Thus, a biologically active variant of a reference aglycosylated PLG polypeptide can differ from the reference amino acid sequence by as few as 1-15 amino acid residues, as few as 1-10 amino acid residues, such as 6-10 amino acid residues, as few as 5 amino acid residues, or as few as 4, 3, 2 or even 1 amino acid residue.

Thus, where the reference aglycosylated PLG polypeptide comprises the mature PLG sequence set forth in SEQ ID NO:4, 16, or 20 with a substitution of the Asn residue at position 289 of this sequence with a residue other than Asn, a biologically active variant of this reference aglycosylated PLG polypeptide will have at least about 50%, 60%, 65%, 70%, more typically at least about 75%, 80%, 85%, preferably at least about 90%, 91%, 92%, 93%, 94%, 95%, 96%, 97%, 98%, 99% or more sequence identity to the amino acid sequence set forth in SEQ ID NO:4, 16, or 20, respectively, and will retain at least the substituted residue at the position corresponding to Ser-289 of SEQ ID NO:4, 16, or 20 and in some embodiments also will retain the serine residue at the position corresponding to Ser-249 of SEQ ID NO:4, 16, or 20 or will also retain the threonine residue at the position corresponding to Thr-346 of SEQ ID NO:4, 16, or 20 or will also retain both the serine residue at the position corresponding to Ser-249 of SEQ ID NO:4, 16, or 20 and the threonine residue at the position corresponding to Thr-346 of SEQ ID NO:4, 16, or 20.

For example, where the reference aglycosylated PLG polypeptide comprises the mature PLG sequence set forth in SEQ ID NO:4 with the N289Q, N289H, N289K, N289R, or N289D substitution, a biologically active variant of this aglycosylated PLG polypeptide will have at least about 50%, 60%, 65%, 70%, more typically at least about 75%, 80%, 85%, preferably at least about 90%, 91%, 92%, 93%, 94%, 95%, 96%, 97%, 98%, 99% or more sequence identity to the amino acid sequence set forth in SEQ ID NO:4, and will retain at least the N289Q, N289H, N289K, N289R, or N289D substitution, and in some embodiments also will retain the serine residue at the position corresponding to Ser-249 of SEQ ID NO:4, or will also retain the threonine residue at the position corresponding to Thr-346 of SEQ ID NO:4, or will also retain both the serine residue at the position corresponding to Ser-249 of SEQ ID NO:4 and the threonine residue at the position corresponding to Thr-346 of SEQ ID NO:4.

In one such embodiment, the reference aglycosylated PLG polypeptide comprises the mature PLG sequence set forth in SEQ ID NO:6 (which differs from SEQ ID NO:4 in having the N289D substitution), and a biologically active variant of this aglycosylated PLG polypeptide will have at least about 50%, 60%, 65%, 70%, more typically at least about 75%, 80%, 85%, preferably at least about 90%, 91%, 92%, 93%, 94%, 95%, 96%, 97%, 98%, 99% or more sequence identity to the amino acid sequence set forth in SEQ ID NO:6, and will retain at least the N289D substitution, and in some embodiments also will retain the serine residue at the position corresponding to Ser-249 of SEQ ID NO:6, or will also retain the threonine residue at the position corresponding to Thr-346 of SEQ ID NO:6, or will also retain both the serine residue at the position corresponding to Ser-249 of SEQ ID NO:6 and the threonine residue at the position corresponding to Thr-346 of SEQ ID NO:6.

The foregoing biologically active variants are exemplified with reference to the mature human PLG polypeptide of SEQ ID NO:4, 16, or 20. Examples of biologically active variants of human PLG are known in the art and are described, e.g., in U.S. Pat. No. 5,190,756. To retain biological activity, any substitutions will preferably be conservative in nature, and truncations and substitutions will generally made in residues that are not required for protease activity. The residues and domains underlying the activity of plasmin/PLG are known in the art and have been described, e.g., in Kolev et al. (1997) J. Biol. Chem. 272: 13666-675; de los Santos et al. (1997) Ciba Found. Symp. 212:66-76, Peisach et al. (1999) Biochemistry 38:11180-11188, and Turner et al. (2002) J. Biol. Chem. 277:33-68-74); each of which is herein incorporated by reference in its entirety.

The comparison of sequences and determination of percent identity and percent similarity between two sequences can be accomplished using a mathematical algorithm. In a preferred embodiment, the percent identity between two amino acid sequences is determined using the Needleman and Wunsch (1970) J. Mol. Biol. 48:444-453 algorithm, which is incorporated into the GAP program in the GCG software package (available at www.accelrys.com), using either a BLOSSUM62 matrix or a PAM250 matrix, and a gap weight of 16, 14, 12, 10, 8, 6, or 4 and a length weight of 1, 2, 3, 4, 5, or 6.

Alternatively, percent identity between two nucleotide sequences can be determined using the GAP program in the GCG software package, using a BLOSUM62 scoring matrix (see Henikoff et al. (1989) Proc. Natl. Acad. Sci. USA 89:10915) and a gap weight of 40, 50, 60, 70, or 80 and a length weight of 1, 2, 3, 4, 5, or 6. A particularly preferred set of parameters (and the one that should be used if the practitioner is uncertain about what parameters should be applied to determine if a molecule is within a sequence identity limitation of the invention) is using a BLOSUM62 scoring matrix with a gap weight of 60 and a length weight of 3.

The percent identity between two amino acid or nucleotide sequences can also be determined using the algorithm of E. Meyers and W. Miller (1989) CABIOS 4:11-17 which has been incorporated into the ALIGN program (version 2.0), using a PAM120 weight residue table, a gap length penalty of 12 and a gap penalty of 4.

Fragments of PLG can be produced from the aglycosylated PLG polypeptides described herein. Such fragments may comprise at least 50, at least 60, at least 70, at least 80, at least 90, at least 95, at least 101, at least 150, at least 200, at least 250, at least 300, at least 350, at least 377, at least 400, at least 450, at least 500, at least 550, at least 600, at least 650, at least 700, or at least 750 contiguous amino acids of the PLG polypeptide. Examples of fragments that may be produced according to the invention include mPLG, miniplasminogen, and angiostatin. Non-limiting examples of plasminogen or microplasminogen fragments are given in O'Reilly et al. (1994) Cell 79:315-28; Sim et al. (1997) Cancer Res. 57:1329-34; U.S. Pat. No. 5,972,896, and U.S. Patent Publications 20020164717, 20020037847, and 20010016644; each of which is herein incorporated by reference in its entirety. In some embodiments, the fragments retain the ability to be activated to a polypeptide having the desired enzymatic activity (i.e., the serine protease activity of plasmin.

Alternatively, fragments of an aglycosylated PLG polypeptide of the invention can be expressed in the plant host of interest. In this manner, an expression construct encoding a fragment of an aglycosylated PLG polypeptide of the invention can be introduced into the plant host to provide for expression of the PLG fragment within the plant host. In such embodiments, the encoded PLG fragment will retain at least the substituted residue at the position corresponding to Ser-289 of SEQ ID NO:4, 16, or 20 and in some embodiments also will retain the serine residue at the position corresponding to Ser-249 of SEQ ID NO:4, 16, or 20, or will also retain the threonine residue at the position corresponding to Thr-346 of SEQ ID NO:4, 16, or 20, or will also retain both the serine residue at the position corresponding to Ser-249 of SEQ ID NO:4, 16, or 20 and the threonine residue at the position corresponding to Thr-346 of SEQ ID NO:4, 16, or 20. In this manner, the expressed PLG fragment is also aglycosylated. Preferably the encoded PLG fragment is biologically active, i.e., it retains the ability to be activated to a polypeptide having the desired enzymatic activity (i.e., the serine protease activity of plasmin.

Modification of PLG-Encoding Nucleic Acid Molecules for Enhanced Expression in Plants

Because the expression systems described below are plant-based, the nucleic acid molecules described herein can be modified to include plant-preferred codons within any portion of the molecule that comprises coding sequence, including the coding sequence for the aglycosylated PLG polypeptides of the invention. Methods are available in the art for synthesizing nucleotide sequences with plant-preferred codons. See, for example, U.S. Pat. Nos. 5,380,831 and 5,436,391; Perlak et al. (1991) Proc. Natl. Acad. Sci. USA 15:3324; Iannacome et al. (1997) Plant Mol. Biol. 34:485; and Murray et al., (1989) Nucleic Acids. Res. 17:477, herein incorporated by reference.

Plant-preferred codons can be determined from the codons of highest frequency in proteins expressed in the plant. Thus, the frequency of usage of a particular codon in the plant can be determined by analyzing codon usage in a group of coding sequences from the plant. A number of plant coding sequences are known to one of ordinary skill in the art. See, e.g., the sequences contained in the GenBank® database, which is available at the website for the National Center for Biotechnology Information. In addition, tables showing frequency of codon usage based on the sequences contained in the most recent GenBank® release can be found on the website for the Kazusa DNA Research Institute in Chiba Japan (see the world wide web at kazusa.or.jp/codon/). This database is described in Nakamura et al. (2000) Nucl. Acids Res. 28: 292.

Nucleic acid sequences have been optimized for expression in specific plants such as duckweed and other monocots. See, e.g., EP 0 359 472, EP 0 385 962, WO 91/16432; Perlak et al. (1991) Proc. Natl. Acad. Sci. USA 88:3324; Iannacome et al. (1997) Plant Mol. Biol. 34:485; and Murray et al. (1989) Nucleic Acids Res. 17:477, and the like, herein incorporated by reference in their entirety. Moreover, all or any part of the nucleic acid sequence can be optimized or synthetic. In other words, fully optimized or partially optimized sequences may also be used. For example, 40%, 45%, 50%, 55%, 60%, 65%, 70%, 75%, 80%, 85%, 87%, 90%, 91%, 92%, 93%, 94%, 95%, 96%, 97%, 98%, 99%, or 100% of the codons may be plant-preferred codons. For example, the nucleic acid molecules of the invention comprising a nucleotide sequence encoding an aglycosylated PLG polypeptide of the invention, or an aglycosylated fragment or variant thereof, can comprise between 50%-100% plant-preferred codons or between 70%-100% plant-preferred codons within the coding sequence for the aglycosylated PLG polypeptide or aglycosylated fragment or variant thereof. Alternatively, between 90%-96% of the codons are plant-preferred codons. The PLG-encoding sequence of the nucleic acid molecule of the invention can comprise codons used with a frequency of at least 17% in the plant.

For example, where the plant host is a member of the duckweed family, the PLG-encoding nucleic acid molecules of the invention can be optimized for expression in duckweed by incorporating duckweed-preferred codons within the nucleotide sequence encoding the aglycosylated PLG polypeptide of interest. Codon usage in the members of the Lemnaceae, such as Lemna gibba (Table 2) and Lemna minor (Table 3), is shown below. Table 1 or 2 can be used to select duckweed-preferred codons for use in the PLG-encoding nucleic acid molecules described herein. For example, the nucleic acid molecule encoding the aglycosylated PLG polypeptide of SEQ ID NO:6, which differs from the mature human PLG of SEQ ID NO:4 in having the N289D substitution, can be optimized for expression in duckweed by modifying the PLG-encoding sequence, for example, the coding sequence set forth in SEQ ID NO:1 or SEQ ID NO:3, to comprise codons used with a frequency of at least 17% in duckweed. One such exemplary duckweed codon-optimized PLG-encoding sequence is shown in SEQ ID NO:5, which encodes the aglycosylated PLG polypeptide of SEQ ID NO:6.

TABLE 2
Lemna gibba codon usage from
GenBank® Release 139.

| Amino Acid | Codon | Number | /1000 | Fraction |
|---|---|---|---|---|
| Gly | GGG | 57.00 | 28.89 | 0.35 |
| Gly | GGA | 8.00 | 4.05 | 0.05 |
| Gly | GGT | 3.00 | 1.52 | 0.02 |
| Gly | GGC | 93.00 | 47.14 | 0.58 |
| Glu | GAG | 123.00 | 62.34 | 0.95 |
| Glu | GAA | 6.00 | 3.04 | 0.05 |
| Asp | GAT | 6.00 | 3.04 | 0.08 |
| Asp | GAC | 72.00 | 36.49 | 0.92 |
| Val | GTC | 62.00 | 31.42 | 0.47 |
| Val | GTA | 0.00 | 0.00 | 0.00 |
| Val | GTT | 18.00 | 9.12 | 0.14 |
| Val | GTC | 51.00 | 25.85 | 0.39 |
| Ala | GCG | 44.00 | 22.30 | 0.21 |
| Ala | GCA | 14.00 | 7.10 | 0.07 |
| Ala | GCT | 14.00 | 7.10 | 0.07 |
| Ala | GCC | 139.00 | 7.45 | 0.66 |
| Arg | AGG | 16.00 | 8.11 | 0.15 |
| Arg | AGA | 11.00 | 5.58 | 0.10 |
| Ser | AGT | 1.00 | 0.51 | 0.01 |
| Ser | AGC | 44.00 | 22.30 | 0.31 |
| Lys | AAG | 116.00 | 58.79 | 1.00 |
| Lys | AAA | 0.00 | 0.00 | 0.00 |
| Asn | AAT | 2.00 | 1.01 | 0.03 |
| Asn | AAC | 70.00 | 35.48 | 0.97 |
| Met | ATG | 67.00 | 33.96 | 1.00 |
| Ile | ATA | 4.00 | 2.03 | 0.06 |
| Ile | ATT | 0.00 | 0.00 | 0.00 |
| Ile | ATC | 63.00 | 31.93 | 0.94 |
| Thr | ACG | 19.00 | 9.63 | 0.25 |
| Thr | ACA | 1.00 | 0.51 | 0.01 |
| Thr | ACT | 6.00 | 3.04 | 0.08 |
| Thr | ACC | 50.00 | 25.34 | 0.66 |
| Trp | TGG | 45.00 | 22.81 | 1.00 |
| Stop Codon | TGA | 4.00 | 2.03 | 0.36 |
| Cys | TGT | 0.00 | 0.00 | 0.00 |
| Cys | TGC | 34.00 | 17.23 | 1.00 |
| Stop Codon | TAG | 0.00 | 0.00 | 0.00 |
| Stop Codon | TAA | 7.00 | 3.55 | 0.64 |
| Tyr | TAT | 4.00 | 2.03 | 0.05 |
| Tyr | TAC | 76.00 | 38.52 | 0.95 |
| Leu | TTG | 5.00 | 2.53 | 0.04 |
| Leu | TTA | 0.00 | 0.00 | 0.00 |
| Phe | TTT | 4.00 | 20.3 | 0.04 |
| Phe | TTC | 92.00 | 46.63 | 0.96 |
| Ser | TCG | 34.00 | 17.23 | 0.24 |
| Ser | TCA | 2.00 | 1.01 | 0.01 |
| Ser | TCT | 1.00 | 0.51 | 0.01 |
| Ser | TCC | 59.00 | 29.90 | 0.42 |
| Arg | CGG | 23.00 | 11.66 | 0.22 |
| Arg | CGA | 3.00 | 1.52 | 0.03 |
| Arg | CGT | 2.00 | 1.01 | 0.02 |
| Arg | CGC | 50.00 | 25.34 | 0.48 |
| Gln | CAG | 59.00 | 29.90 | 0.86 |
| Gln | CAA | 10.00 | 5.07 | 0.14 |
| His | CAT | 5.00 | 2.53 | 0.26 |
| His | CAC | 14.00 | 7.10 | 0.74 |
| Leu | CTG | 43.00 | 21.71 | 0.35 |
| Leu | CTA | 2.00 | 1.01 | 0.02 |
| Leu | CTT | 1.00 | 0.51 | 0.01 |
| Leu | CTC | 71.00 | 35.99 | 0.58 |
| Pro | CCG | 44.00 | 22.30 | 0.31 |
| Pro | CCA | 6.00 | 3.04 | 0.04 |
| Pro | CCT | 13.00 | 6.59 | 0.09 |
| Pro | CCC | 80.00 | 40.55 | 0.56 |

TABLE 3
Lemna minor codon usage from
GenBank® Release 139.

| Amino Acid | Codon | Number | /1000 | Fraction |
|---|---|---|---|---|
| Gly | GGG | 8.00 | 17.39 | 0.22 |
| Gly | GGA | 11.00 | 23.91 | 0.31 |
| Gly | GGT | 1.00 | 2.17 | 0.03 |
| Gly | GGC | 16.00 | 34.78 | 0.44 |
| Glu | GAG | 25.00 | 54.35 | 0.78 |
| Glu | GAA | 7.00 | 15.22 | 0.232 |
| Asp | GAT | 8.00 | 17.39 | 0.33 |
| Asp | GAC | 16.00 | 34.78 | 0.67 |
| Val | GTC | 21.00 | 45.65 | 0.53 |
| Val | GTA | 3.00 | 6.52 | 0.07 |
| Val | GTT | 6.00 | 13.04 | 0.15 |
| Val | GTC | 10.00 | 21.74 | 0.25 |
| Ala | GCG | 13.00 | 28.26 | 0.32 |
| Ala | GCA | 8.00 | 17..39 | 0.20 |
| Ala | GCT | 6.00 | 13.04 | 0.15 |
| Ala | GCC | 14.00 | 30.43 | 0.34 |
| Arg | AGG | 9.00 | 19.57 | 0.24 |
| Arg | AGA | 11.00 | 23.91 | 0.30 |
| Ser | AGT | 2.00 | 4.35 | 0.05 |
| Ser | AGC | 11.00 | 23.91 | 0.26 |
| Lys | AAG | 13.00 | 28.26 | 0.68 |
| Lys | AAA | 6.00 | 13.04 | 0.32 |
| Asn | AAT | 0.00 | 0.00 | 0.00 |
| Asn | AAC | 12.00 | 26.09 | 1.00 |
| Met | ATG | 9.00 | 19.57 | 1.00 |
| Ile | ATA | 1.00 | 2.17 | 0.08 |
| Ile | ATT | 2.00 | 4.35 | 0.15 |
| Ile | ATC | 10.00 | 21.74 | 0.77 |
| Thr | ACG | 5.00 | 10.87 | 0.28 |
| Thr | ACA | 2.00 | 4.35 | 0.11 |
| Thr | ACT | 2.00 | 4.35 | 0.11 |
| Thr | ACC | 9.00 | 19.57 | 0.50 |
| Trp | TGG | 8.00 | 17.39 | 1.00 |
| Stop Codon | TGA | 1.00 | 2.17 | 1.00 |
| Cys | TGT | 1.00 | 2.17 | 0.12 |
| Cys | TGC | 7.00 | 15.22 | 0.88 |
| Stop Codon | TAG | 0.00 | 0.00 | 0.00 |
| Stop Codon | TAA | 0.00 | 0.00 | 0.00 |
| Tyr | TAT | 1.00 | 2.17 | 0.12 |
| Tyr | TAC | 7.00 | 15.22 | 0.88 |
| Leu | TTG | 3.00 | 6.52 | 0.08 |
| Leu | TTA | 1.00 | 2.17 | 0.03 |
| Phe | TTT | 6.00 | 13.04 | 0.25 |
| Phe | TTC | 18.00 | 39.13 | 0.75 |
| Ser | TCG | 11.0 | 23.91 | 0.26 |
| Ser | TCA | 4.00 | 8.70 | 0.09 |
| Ser | TCT | 6.00 | 13.04 | 0.14 |
| Ser | TCC | 9.00 | 19.57 | 0.21 |
| Arg | CGG | 4.00 | 8.70 | 0.11 |
| Arg | CGA | 4.00 | 8.70 | 0.11 |
| Arg | CGT | 0.00 | 0.00 | 0.00 |
| Arg | CGC | 9.00 | 19.57 | 0.24 |
| Gln | CAG | 11.0 | 23.91 | 0.73 |
| Gln | CAA | 4.00 | 8.70 | 0.27 |
| His | CAT | 0.00 | 0.00 | 0.00 |
| His | CAC | 6.00 | 13.04 | 1.00 |
| Leu | CTG | 9.00 | 19.57 | 0.24 |
| Leu | CTA | 4.00 | 8.70 | 0.11 |
| Leu | CTT | 4.00 | 8.70 | 0.11 |
| Leu | CTC | 17.00 | 36.96 | 0.45 |
| Pro | CCG | 8.00 | 17.39 | 0.29 |
| Pro | CCA | 7.00 | 15.22 | 0.25 |
| Pro | CCT | 5.00 | 10.87 | 0.18 |
| Pro | CCC | 8.00 | 17.39 | 0.29 |

Other modifications also can be made to the PLG-encoding nucleic acid molecules described herein to enhance their expression in plants. These modifications include, but are not limited to, elimination of sequences encoding spurious polyadenylation signals, exon-intron splice site signals, transposon-like repeats, and other such well characterized sequences that may be deleterious to gene expression. The G-C content of the sequence may be adjusted to levels average for a given plant, as calculated by reference to known genes expressed in the plant cell. When possible, the PLG-encoding nucleic acid molecules can be modified to avoid predicted hairpin secondary mRNA structures.

There are known differences between the optimal translation initiation context nucleic acid sequences for translation initiation codons in animals and plants and the composition of these translation initiation context nucleic acid sequences can influence the efficiency of translation initiation. See, e.g., Lukaszewicz et al. (2000) Plant Science 154:89-98; and Joshi et al. (1997) Plant Mol. Biol. 35:993-1001. Thus, the translation initiation context nucleotide sequence for the translation initiation codon of the PLG-encoding nucleic acid molecules described herein can be modified to enhance expression in the plant host of interest, for example duckweed. Thus, for example, the PLG-encoding nucleic acid molecules described herein can be modified such that the three nucleic acids directly upstream of the translation initiation codon of the PLG-encoding sequences described herein can be “ACC.” Alternatively, these three nucleic acids can be “ACA.”

Expression of a transgene in a plant host of interest, for example, duckweed, can also be enhanced by the use of 5′ leader sequences. Such leader sequences can act to enhance translation. One or more leader sequences may be used in combination to enhance expression of the target nucleotide sequence. Translation leaders are known in the art and include, but are not limited to, picornavirus leaders, e.g., EMCV leader (Encephalomyocarditis 5′ noncoding region; Elroy-Stein et al. (1989) Proc. Natl. Acad. Sci USA 86:6126); potyvirus leaders, e.g., TEV leader (Tobacco Etch Virus; Allison et al. (1986) Virology 154:9); human immunoglobulin heavy-chain binding protein (BiP; Macajak and Sarnow (1991) Nature 353:90); untranslated leader from the coat protein mRNA of alfalfa mosaic virus (AMV RNA 4; Jobling and Gehrke (1987) Nature 325:622); tobacco mosaic virus leader (TMV; Gallie (1989) Molecular Biology of RNA, 23:56); potato etch virus leader (Tomashevskaya et al. (1993) J. Gen. Virol. 74:2717-2724); Fed-1 5′ untranslated region (Dickey (1992) EMBO J. 11:2311-2317); RbcS 5′ untranslated region (Silverthorne et al. (1990) J. Plant. Mol. Biol. 15:49-58); and maize chlorotic mottle virus leader (MCMV; Lommel et al. (1991) Virology 81:382). See also, Della-Cioppa et al. (1987) Plant Physiology 84:965. Leader sequence comprising plant intron sequence, including intron sequence from the maize dehydrogenase 1 gene, the castor bean catalase gene, or the Arabidopsis tryptophan pathway gene PATI has also been shown to increase translational efficient in plants (Callis et al. (1987) Genes Dev. 1:1183-1200; Mascarenhas et al. (1990) Plant Mol. Biol. 15:913-920). In one embodiment of the present invention, nucleotide sequence corresponding to nucleotides 1222-1775 of the maize alcohol dehydrogenase 1 gene (GenBank Accession Number X04049), set forth in SEQ ID NO:9, is inserted upstream of the PLG-encoding nucleotide sequence of interest to enhance the efficiency of its translation. In another embodiment, the expression vector contains the leader from the Lemna gibba ribulose-bis-phosphate carboxylase small subunit 5B gene (Buzby et al. (1990) Plant Cell 2:805-814) (see SEQ ID NO:10).

It is recognized that any of the plant expression-enhancing nucleotide sequence modifications described above can be used in the present invention, including any single modification or any possible combination of modifications. The phrase “modified for enhanced expression” in a plant, such as a duckweed plant, as used herein refers to a nucleotide sequence that contains any one or any combination of these modifications.

Expression Cassettes

The present invention also includes expression cassettes in which the PLG-encoding nucleic acid molecules described herein can be operably linked to various expression control sequences. The expression cassette can be provided with a plurality of restriction sites for insertion of the nucleic acid molecules described herein.

As used herein, “expression cassette,” “expression construct,” “vector construct,” “expression vector,” or “gene expression vector” all refer to an assembly that is capable of directing expression of a nucleic acid sequence of interest, such as a nucleic acid sequence encoding an aglycosylated PLG polypeptide of the present invention.

As used herein, “operably linked” means that elements of an expression cassette are configured so as to perform their usual function. Thus, control sequences (i.e., promoters) operably linked to a coding sequence can be capable of effecting expression of the coding sequence. The control sequences need not be contiguous with the coding sequence, so long as they function to direct the expression thereof. Thus, for example, intervening untranslated, yet transcribed, sequences can be present between a promoter and a coding sequence, and the promoter sequence can still be considered “operably linked to the coding sequence.

As used herein, “control sequence,” “control sequences,” “expression control sequence” or “expression control sequences” means promoter sequences, polyadenylation signals, transcription termination sequences, upstream regulatory domains, origins of replication, internal ribosome entry sites (“IRES”), enhancers, and the like, which collectively provide for replication, transcription, and translation of a coding sequence in a recipient cell. Not all of these control sequences need always be present so long as the selected coding sequence is capable of being replicated, transcribed, and translated in an appropriate host cell. Expression control sequences therefore can be a regulatory region of DNA usually comprising a TATA box capable of directing RNA polymerase II to initiate RNA synthesis at the appropriate transcription initiation site for a particular coding sequence. An expression control sequence can additionally comprise other recognition sequences generally positioned upstream or 5′ to the TATA box, which influence (e.g., enhance) the transcription initiation rate. Furthermore, an expression control sequence may additionally comprise sequences generally positioned downstream or 3′ to the TATA box, which influence (e.g., enhance) the transcription initiation rate.

As used herein, a “promoter” means a nucleotide region comprising a nucleic acid (i.e., DNA) regulatory sequence, wherein the regulatory sequence is derived from a gene that is capable of binding RNA polymerase and initiating transcription of a downstream (3′-direction) coding sequence. Transcription promoters can include “inducible promoters” (where expression of a polynucleotide sequence operably linked to the promoter is induced by an analyte, cofactor, regulatory protein, etc.), “repressible promoters” (where expression of a polynucleotide sequence operably linked to the promoter is repressed by an analyte, cofactor, regulatory protein, etc.) and “constitutive promoters” (where expression of a polynucleotide sequence operably linked to the promoter is unregulated and therefore continuous).

The transcriptional initiation region, such as the promoter, of the expression cassette can be native or homologous or foreign or heterologous to its intended host cell, or could be the natural sequence or a synthetic sequence. By foreign, it is intended that the transcriptional initiation region is not found in the wild-type host into which the transcriptional initiation region is introduced.

Any suitable promoter known in the art can be employed according to the present invention (including bacterial, yeast, fungal, insect, mammalian, and plant promoters). For example, plant promoters, including duckweed promoters, may be used. Exemplary promoters include, but are not limited to, the Cauliflower Mosaic Virus 35S promoter, the opine synthetase promoters (e.g., nos, mas, ocs, etc.), the ubiquitin promoter, the actin promoter, the ribulose bisphosphate (RubP) carboxylase small subunit promoter, and the alcohol dehydrogenase promoter. The duckweed RubP carboxylase small subunit promoter is known in the art (Silverthorne et al. (1990) Plant Mol. Biol. 15:49). Other promoters from viruses that infect plants, preferably duckweed, are also suitable including, but not limited to, promoters isolated from Dasheen mosaic virus, Chlorella virus (e.g., the Chlorella virus adenine methyltransferase promoter; Mitra et al. (1994) Plant Mol. Biol. 26:85), tomato spotted wilt virus, tobacco rattle virus, tobacco necrosis virus, tobacco ring spot virus, tomato ring spot virus, cucumber mosaic virus, peanut stump virus, alfalfa mosaic virus, sugarcane baciliform badnavirus and the like. See also the expression control elements (promoters and introns) isolated from the Lemnaceae ubiquitin, r-histone, and chitinase genes, as described in copending and commonly owned U.S. Patent Application Publication No. 20070180583, herein incorporated by reference in its entirety.

Finally, promoters can be chosen to give a desired level of regulation. For example, in some instances, one can use a promoter that confers constitutive expression (e.g., the mannopine synthase promoter from Agrobacterium tumefaciens). Alternatively, one can use promoters that are activated in response to specific environmental stimuli (e.g., heat shock gene promoters, drought-inducible gene promoters, pathogen-inducible gene promoters, wound-inducible gene promoters and light/dark-inducible gene promoters) or plant growth regulators (e.g., promoters from genes induced by abscissic acid, auxins, cytokinins and gibberellic acid) or other compounds such as ethanol or ethylene. As a further alternative, one can use a promoter that gives tissue-specific expression (e.g., root, leaf and floral-specific promoters).

The overall strength of a given promoter can be influenced by the combination and spatial organization of cis-acting nucleotide sequences such as upstream activating sequences. For example, activating nucleotide sequences derived from the A. tumefaciens octopine synthase gene can enhance transcription from the Agrobacterium tumefaciens mannopine synthase promoter (see, U.S. Pat. No. 5,955,646, incorporated herein by reference as if set forth in its entirety).

The expression cassette also can contain activating nucleotide sequences inserted upstream of the promoter to enhance the expression of the nucleic acid sequence of interest. For example, the expression cassette can include three upstream activating sequences derived from the A. tumefaciens octopine synthase gene operably linked to a promoter derived from an A. tumefaciens mannopine synthase gene (see U.S. Pat. No. 5,955,646, herein incorporated by reference).

Expression cassettes therefore can include in the 5′-3′ direction of transcription, a transcriptional and translational initiation region, a PLG-encoding nucleic acid molecule of the invention, and a transcriptional and translational termination region functional in plants. Any suitable termination sequence known in the art may be used in accordance with the present invention. The termination region may be native with the transcriptional initiation region, may be native with the nucleotide sequence of interest, or may be derived from another source. Convenient termination regions are available from the Ti-plasmid of A. tumefaciens, such as the octopine synthetase and nopaline synthetase termination regions. See also Guerineau et al. (1991) Mol. Gen. Genet. 262:141; Proudfoot (1991) Cell 64:671; Sanfacon et al. (1991) Genes Dev. 5:141; Mogen et al. (1990) Plant Cell 2:1261; Munroe et al. (1990) Gene 91:151; Ballas et al. (1989) Nucleic Acids Res. 17:7891; and Joshi et al. (1987) Nucleic Acids Res. 15:9627. Additional exemplary termination sequences are the pea RubP carboxylase small subunit termination sequence and the Cauliflower Mosaic Virus 35S termination sequence and the ubiquitin terminator from many plant species. Other suitable termination sequences will be apparent to those skilled in the art.

The expression cassettes can contain more than one gene or nucleic acid sequence to be transferred and expressed in the transformed plant host, so long as at least one of the nucleic acid sequences encodes an aglycosylated PLG polypeptide. As such, each nucleic acid sequence can be operably linked to 5′ and 3′ regulatory sequences. Alternatively, multiple expression cassettes can be provided.

Generally, the expression cassette will comprise a selectable marker gene for the selection of transformed cells or tissues. Selectable marker genes include genes encoding antibiotic resistance, such as those encoding neomycin phosphotransferase II (NEO), neomycin phosphotransferase III and hygromycin phosphotransferase (HPT), as well as genes conferring resistance to herbicidal compounds. Herbicide resistance genes generally code for a modified target protein insensitive to the herbicide or for an enzyme that degrades or detoxifies the herbicide in the plant before it can act. See DeBlock et al. (1987) EMBO J. 6:2513; DeBlock et al. (1989) Plant Physiol. 91:691; Fromm et al. (1990) BioTechnology 8:833; Gordon-Kamm et al. (1990) Plant Cell 2:603; and Frisch et al. (1995) Plant Mol. Biol. 27:405-9. For example, resistance to glyphosphate or sulfonylurea herbicides has been obtained using genes coding for the mutant target enzymes, 5-enolpyruvylshikimate-3-phosphate synthase (EPSPS) and acetolactate synthase (ALS). Resistance to glufosinate ammonium, boromoxynil, and 2,4-dichlorophenoxyacetate (2,4-D) have been obtained by using bacterial genes encoding phosphinothricin acetyltransferase, a nitrilase, or a 2,4-dichlorophenoxyacetate monooxygenase, which detoxify the respective herbicides.

For purposes of the present invention, selectable marker genes include, but are not limited to, genes encoding neomycin phosphotransferase II (Fraley et al. (1986) CRC Critical Reviews in Plant Science 4:1), neomycin phosphotransferase III (Frisch et al. (1995) Plant Mol. Biol. 27:405-9), cyanamide hydratase (Maier-Greiner et al. (1991) Proc. Natl. Acad. Sci. USA 88:4250); aspartate kinase; dihydrodipicolinate synthase (Perl et al. (1993) Bio Technology 11:715); bar gene (Toki et al. (1992) Plant Physiol. 100:1503; Meagher et al. (1996) Crop Sci. 36:1367); tryptophan decarboxylase (Goddijn et al. (1993) Plant Mol. Biol. 22:907); neomycin phosphotransferase (NEO; Southern et al. (1982) J. Mol. Appl. Gen. 1:327); hygromycin phosphotransferase (HPT or HYG; Shimizu et al. (1986) Mol. Cell. Biol. 6:1074); dihydrofolate reductase (DHFR; Kwok et al. (1986) Proc. Natl. Acad. Sci. USA 83:4552); phosphinothricin acetyltransferase (DeBlock et al. (1987) EMBO J. 6:2513); 2,2-dichloropropionic acid dehalogenase (Buchanan-Wollatron et al. (1989) J. Cell. Biochem. 13D:330); acetohydroxyacid synthase (U.S. Pat. No. 4,761,373 to Anderson et al.; Haughn et al. (1988) Mol. Gen. Genet. 221:266); 5-enolpyruvyl-shikimate-phosphate synthase (aroA; Comai et al. (1985) Nature 317:741); haloarylnitrilase (WO 87/04181 to Stalker et al.); acetyl-coenzyme A carboxylase (Parker et al. (1990) Plant Physiol. 92:1220); dihydropteroate synthase (sulI; Guerineau et al. (1990) Plant Mol. Biol. 15:127); and 32 kDa photosystem II polypeptide (psbA; Hirschberg et al. (1983) Science 222:1346 (1983).

Also included are genes encoding resistance to: gentamycin (e.g., aacC1, Wohlleben et al. (1989) Mol. Gen. Genet. 217:202-208); chloramphenicol (Herrera-Estrella et al. (1983) EMBO J. 2:987); methotrexate (Herrera-Estrella et al. (1983) Nature 303:209; Meijer et al. (1991) Plant Mol. Biol. 16:807); hygromycin (Waldron et al. (1985) Plant Mol. Biol. 5:103; Zhijian et al. (1995) Plant Science 108:219; Meijer et al. (1991) Plant Mol. Bio. 16:807); streptomycin (Jones et al. (1987) Mol. Gen. Genet. 210:86); spectinomycin (Bretagne-Sagnard et al. (1996) Transgenic Res. 5:131); bleomycin (Hille et al. (1986) Plant Mol. Biol. 7:171); sulfonamide (Guerineau et al. (1990) Plant Mol. Bio. 15:127); bromoxynil (Stalker et al. (1988) Science 242:419); 2,4-D (Streber et al. (1989) BioTechnology 7:811); phosphinothricin (DeBlock et al. (1987) EMBO J. 6:2513); spectinomycin (Bretagne-Sagnard and Chupeau, Transgenic Research 5:131).

The bar gene confers herbicide resistance to glufosinate-type herbicides, such as phosphinothricin (PPT) or bialaphos, and the like. As noted above, other selectable markers that could be used in the vector constructs include, but are not limited to, the pat gene, also for bialaphos and phosphinothricin resistance, the ALS gene for imidazolinone resistance, the HPH or HYG gene for hygromycin resistance, the EPSP synthase gene for glyphosate resistance, the Hml gene for resistance to the Hc-toxin, and other selective agents used routinely and known to one of ordinary skill in the art. See, Yarranton (1992) Curr. Opin. Biotech. 3:506; Chistopherson et al. (1992) Proc. Natl. Acad. Sci. USA 89:6314; Yao et al. (1992) Cell 71:63; Reznikoff (1992) Mol. Microbiol. 6:2419; Barkley et al. (1980) The Operon 177-220; Hu et al. (1987) Cell 48:555; Brown et al. (1987) Cell 49:603; Figge et al. (1988) Cell 52:713; Deuschle et al. (1989) Proc. Natl. Acad. Sci. USA 86:5400; Fuerst et al. (1989) Proc. Natl. Acad. Sci. USA 86:2549; Deuschle et al. (1990) Science 248:480; Labow et al. (1990) Mol. Cell. Biol. 10:3343; Zambretti et al. (1992) Proc. Natl. Acad. Sci. USA 89:3952; Baim et al. (1991) Proc. Natl. Acad. Sci. USA 88:5072; Wyborski et al. (1991) Nucleic Acids Res. 19:4647; Hillenand-Wissman (1989) Topics in Mol. And Struc. Biol. 10:143; Degenkolb et al. (1991) Antimicrob. Agents Chemother. 35:1591; Kleinschnidt et al. (1988) Biochemistry 27:1094; Gatz et al. (1992) Plant J. 2:397; Gossen et al. (1992) Proc. Natl. Acad. Sci. USA 89:5547; Oliva et al. (1992) Antimicrob. Agents Chemother. 36:913; Hlavka et al. (1985) Handbook of Experimental Pharmacology 78; and Gill et al. (1988) Nature 334:721; each of which is incorporated herein by reference as if set forth in its entirety. The above list of selectable marker genes is not meant to be limiting. Any lethal or non-lethal selectable marker gene also can be used in the present invention.

Signal Peptides

Because PLG is a secreted protein, it is usually translated from precursor polypeptides that include a “signal peptide” that interacts with a receptor protein on the membrane of the endoplasmic reticulum (ER) to direct the translocation of the growing polypeptide chain across the membrane and into the endoplasmic reticulum for secretion from the cell. This signal peptide is often cleaved from the precursor polypeptide to produce a “mature” polypeptide lacking the signal peptide. Thus, in an embodiment of the present invention, an aglycosylated PLG polypeptide of the invention is expressed in the plant host of interest from a PLG-encoding nucleotide sequence that is operably linked with a nucleotide sequence encoding a signal peptide that directs secretion of the aglycosylated PLG polypeptide.

“Secretion” as used herein refers to translocation of a polypeptide across the plasma membrane of a plant host cell. In some embodiments of the present invention, the aglycosylated PLG polypeptide is retained within the apoplast, the region between the plasma membrane and the cell wall. In other embodiments, the aglycosylated PLG polypeptide is translocated across the cell wall of the plant host cell. Thus, in some embodiments, the aglycosylated PLG polypeptide is secreted into the plant culture medium.

One of ordinary skill in the art is familiar with plant signal peptides that target protein translocation to the endoplasmic reticulum (for secretion outside of the cell). See, for example, U.S. Pat. No. 6,020,169 to Lee et al. Any plant signal peptide can be used to target polypeptide expression to the endoplasmic reticulum. For example, the signal peptide sequence can be the Arabidopsis thaliana basic endochitinase signal peptide, the extensin signal peptide (Stiefel et al. (1990) Plant Cell 2:785-793) or the rice α-amylase signal peptide (SEQ ID NO:8; amino acids 1-31 of NCBI Protein Accession No. AAA33885; see also, Int'l Patent Application Publication No. WO 2007/124186, incorporated herein by reference as if set forth in its entirety). Alternatively, the signal peptide can correspond to a signal peptide of a secreted protein from the plant host of interest, for example, a duckweed plant signal peptide. See, for example, the Lemnaceae chitinase signal peptide disclosed in copending and commonly owned U.S. Patent Application Publication No. 20070180583 (set forth in SEQ ID NO:16, and encoded by SEQ ID NO:15, of that published application).

Alternatively still, a mammalian signal peptide can be used to target recombinant polypeptides expressed in genetically engineered duckweed for secretion. It has been demonstrated that plant cells recognize mammalian signal peptides that target the endoplasmic reticulum, and that these signal peptides can direct the secretion of polypeptides not only through the plasma membrane but also through the plant cell wall. See, U.S. Pat. Nos. 5,202,422 and 5,639,947 to Hiatt et al.

As noted above, the nucleic acid sequence encoding the signal peptide can be modified for enhanced expression in the plant host of interest, for example, duckweed, utilizing any modification or combination of modifications disclosed above for the PLG-encoding nucleic acid molecules described above. For example, a duckweed optimized sequence encoding the signal peptide from rice α-amylase is shown in SEQ ID NO:7. This sequence contains approximately 93% duckweed-preferred codons.

The aglycosylated PLG polypeptide or fragment or variant thereof can be harvested from the plant host material, the culture medium, or the plant host material and the culture medium by any conventional means known in the art and purified by chromatography, electrophoresis, dialysis, solvent-solvent extraction, and the like. See also the purification methods disclosed in the Experimental section herein below.

Plant-Based Expression Systems and Transgenic Plants

The present invention further includes plant-based expression systems and transgenic plants expressing an aglycosylated PLG polypeptide of the invention. Any plant host of interest can be used to express the aglycosylated PLG polypeptides of the invention, including any plant that is amenable to transformation.

As used herein, “plant” means whole plants, plant organs (e.g., leaves, stems, roots, etc.), seeds, plant cells, and progeny of same. Parts of transgenic plants are to be understood within the scope of the invention to comprise, e.g., plant cells, plant protoplasts, plant cell tissue cultures from which plants can be regenerated, tissues, plant calli, embryos as well as flowers, ovules, stems, fruits, leaves, roots, root tips, nodules, and the like originating in transgenic plants or their progeny previously transformed with a DNA molecule of the invention and therefore consisting at least in part of transgenic cells. As used herein, the term “plant cell” means cells of seeds, embryos, ovules, meristematic regions, callus tissue, leaves, fronds, roots, nodules, shoots, gametophytes, sporophytes, anthers, pollen, and microspores.

The class of plants that can be used to express the aglycosylated PLG polypeptides of the invention, and aglycosylated, biologically active fragments and variants thereof, is generally as broad as the class of higher plants amenable to transformation techniques, including both monocotyledonous (monocot) and dicotyledonous (dicot) plants. Examples of dicots include, but are not limited to, legumes including soybeans and alfalfa, tobacco, potatoes, tomatoes, and the like. Examples of monocots include, but are not limited to, maize, rice, oats, barley, wheat, members of the duckweed family, grasses, and the like. In some embodiments, the plant of interest is a member of the duckweed family of plants.

As used herein, “duckweed” means members of the family Lemnaceae. Duckweeds are sole members of the monocotyledonous family Lemnaceae. The Lemnaceae family is composed of five genera and thirty-eight species and are all small, free-floating, fresh-water plants whose geographical range spans the entire globe (Landolt (1986) Biosystematic Investigation on the Family of Duckweeds: The Family of Lemnaceae—A Monograph Study (Geobatanischen Institut ETH, Stiftung Rubel, Zurich)). Although the most morphologically reduced plants known, most duckweed species have all the tissues and organs of much larger plants, including roots, stems, flowers, seeds and fronds. Duckweed species are extensively studied, and a substantial literature exists detailing their ecology, systematics, life-cycle, metabolism, disease and pest susceptibility, as well as their reproductive biology, genetic structure and cell biology (Hillman (1961) Bot. Review 27:221; Landolt (1986) Biosystematic Investigation on the Family of Duckweeds: The Family of Lemnaceae—A Monograph Study (Geobatanischen Institut ETH, Stiftung Rubel, Zurich)).

This family currently is divided into five genera and thirty-eight species of duckweed as follows: genus Lemna (L. aequinoctialis, L. disperma, L. ecuadoriensis, L. gibba, L. japonica, L. minor, L. miniscula, L. obscura, L. perpusilla, L. tenera, L. trisulca, L. turionifera, L. valdiviana); genus Spirodela (S. intermedia, S. polyrrhiza, S. punctata); genus Wolffia (Wa. angusta, Wa. arrhiza, Wa. australina, Wa. borealis, Wa. brasiliensis, Wa. columbiana, Wa. elongata, Wa. globosa, Wa. microscopica, Wa. neglecta); genus Wolfiella (Wl. caudata, Wl. denticulata, Wl. gladiata, Wl. hyalina, Wl. lingulata, Wl. repunda, Wl. rotunda, and Wl. neotropica) and genus Landoltia (L. punctata). Any other genera or species of Lemnaceae, if they exist, are also aspects of the present invention. Lemna species can be classified using the taxonomic scheme described by Landolt, supra.

As used herein, “duckweed nodule” means duckweed tissue comprising duckweed cells where at least about 50%, 55%, 60%, 65%, 70%, 75%, 80%, 85%, 90%, 95% or 100% of the cells are differentiated cells. As used herein, “differentiated cell,” means a cell with at least one phenotypic characteristic (e.g., a distinctive cell morphology or the expression of a marker nucleic acid or protein) that distinguishes it from undifferentiated cells or from cells found in other tissue types. The differentiated cells of the duckweed nodule culture described herein form a tiled smooth surface of interconnected cells fused at their adjacent cell walls, with nodules that have begun to organize into frond primordium scattered throughout the tissue. The surface of the tissue of the nodule culture has epidermal cells connected to each other via plasmadesmata.

The growth habit of the duckweeds is ideal for microbial culturing methods. The plant rapidly proliferates through vegetative budding of new fronds, in a macroscopic manner analogous to asexual propagation in yeast. This proliferation occurs by vegetative budding from meristematic cells. The meristematic region is small and is found on the ventral surface of the frond. Meristematic cells lie in two pockets, one on each side of the frond midvein. The small midvein region is also the site from which the root originates and the stem arises that connects each frond to its mother frond. The meristematic pocket is protected by a tissue flap. Fronds bud alternately from these pockets. Doubling times vary by species and are as short as 20-24 hours (Landolt (1957) Ber. Schweiz. Bot. Ges. 67:271; Chang et al. (1977) Bull. Inst. Chem. Acad. Sin. 24:19; Datko and Mudd (1970) Plant Physiol. 65:16; Venkataraman et al. (1970) Z. Pflanzenphysiol. 62: 316). Intensive culture of duckweed results in the highest rates of biomass accumulation per unit time (Landolt and Kandeler (1987) The Family of Lemnaceae—A Monographic Study Vol. 2: Phytochemistry, Physiology, Application, Bibliography (Veroffentlichungen des Geobotanischen Institutes ETH, Stiftung Rubel, Zurich)), with dry weight accumulation ranging from 6-15% of fresh weight (Tillberg et al. (1979) Physiol. Plant. 46:5; Landolt (1957) Ber. Schweiz. Bot. Ges. 67:271; Stomp, unpublished data). Protein content of a number of duckweed species grown under varying conditions has been reported to range from 15-45% dry weight (Chang et al. (1977) Bull. Inst. Chem. Acad. Sin. 24:19; Chang and Chui (1978) Z. Pflanzenphysiol. 89:91; Porath et al. (1979) Aquatic Botany 7:272; Appenroth et al. (1982) Biochem. Physiol. Pflanz. 177:251). Using these values, the level of protein production per liter of medium in duckweed is on the same order of magnitude as yeast gene expression systems.

The present invention thus provides transformed plants expressing an aglycosylated PLG polypeptide of the present invention, or an aglycosylated fragment or variant thereof. The transformed plants of the invention can be obtained by introducing an expression construct comprising a PLG-encoding nucleic acid molecule of the invention into the plant host of interest, for example, a dicot plant, or a monocot plant, such as duckweed.

The term “introducing” in the context of a polynucleotide, for example, an expression construct comprising a PLG-encoding nucleic acid molecule of the invention, is intended to mean presenting to the plant the polynucleotide in such a manner that the polynucleotide gains access to the interior of a cell of the plant. Where more than one polynucleotide is to be introduced, these polynucleotides can be assembled as part of a single nucleotide construct, or as separate nucleotide constructs, and can be located on the same or different transformation vectors. Accordingly, these polynucleotides can be introduced into the plant host cell of interest in a single transformation event, in separate transformation events, or, for example, as part of a breeding protocol. The compositions and methods of the invention do not depend on a particular method for introducing one or more polynucleotides into a plant, only that the polynucleotide(s) gains access to the interior of at least one cell of the plant. Methods for introducing polynucleotides into plants are known in the art including, but not limited to, transient transformation methods, stable transformation methods, and virus-mediated methods.

“Transient transformation” in the context of a polynucleotide such as the PLG-encoding nucleic acid molecules of the invention, is intended to mean that a polynucleotide is introduced into the plant and does not integrate into the genome of the plant.

By “stably introducing” or “stably introduced” in the context of a polynucleotide (such as the PLG-encoding nucleic acid molecules of the invention) introduced into a plant is intended the introduced polynucleotide is stably incorporated into the plant genome, and thus the plant is stably transformed with the polynucleotide.

“Stable transformation” or “stably transformed” is intended to mean that a polynucleotide, for example, a PLG-encoding nucleic acid molecule described herein, introduced into a plant integrates into the genome of the plant and is capable of being inherited by the progeny thereof, more particularly, by the progeny of multiple successive generations. In some embodiments, successive generations include progeny produced vegetatively (i.e., asexual reproduction), for example, with clonal propagation. In other embodiments, successive generations include progeny produced via sexual reproduction.

An expression construct comprising a PLG-encoding nucleic acid molecule of the invention can be introduced into a plant host of interest using any transformation protocol known to those of skill in art. Transformation protocols as well as protocols for introducing nucleotide sequences into plants may vary depending on the type of plant or plant cell or nodule, that is, monocot or dicot, targeted for transformation. Suitable methods of introducing nucleotide sequences into plants or plant cells or nodules include microinjection (Crossway et al. (1986) Biotechniques 4:320-334), electroporation (Riggs et al. (1986) Proc. Natl. Acad. Sci. USA 83:5602-5606), Agrobacterium-mediated transformation (U.S. Pat. Nos. 5,563,055 and 5,981,840, both of which are herein incorporated by reference), direct gene transfer (Paszkowski et al. (1984) EMBO J. 3:2717-2722), ballistic particle acceleration (see, e.g., U.S. Pat. Nos. 4,945,050; 5,879,918; 5,886,244; and 5,932,782 (each of which is herein incorporated by reference); and Tomes et al. (1995) “Direct DNA Transfer into Intact Plant Cells via Microprojectile Bombardment,” in Plant Cell, Tissue, and Organ Culture: Fundamental Methods, ed. Gamborg and Phillips (Springer-Verlag, Berlin); McCabe et al. (1988) Biotechnology 6:923-926). The cells that have been transformed may be grown into plants in accordance with conventional ways. See, for example, McCormick et al. (1986) Plant Cell Reports 5:81-84.

Stably transformed duckweed can obtained by any gene transfer method known in the art, such as one of the gene transfer methods disclosed in U.S. Pat. No. 6,040,498 or U.S. Patent Application Publication Nos. 2003/0115640, 2003/0033630 or 2002/0088027; each of which is incorporated herein by reference as if set forth in its entirety. Duckweed plant or nodule cultures can be efficiently transformed with an expression cassette containing a nucleic acid sequence as described herein by any one of a number of methods including Agrobacterium-mediated gene transfer, ballistic bombardment or electroporation. The Agrobacterium used can be Agrobacterium tumefaciens or Agrobacterium rhizogenes. Stable duckweed transformants can be isolated by transforming the duckweed cells with both the nucleic acid sequence of interest and a gene that confers resistance to a selection agent, followed by culturing the transformed cells in a medium containing the selection agent. See, for example, U.S. Pat. No. 6,040,498, the contents of which are herein incorporated by reference in their entirety.

The stably transformed plants utilized in these methods should exhibit normal morphology and be fertile by sexual reproduction. Preferably, transformed plants of the present invention contain a single copy of the transferred nucleic acid comprising a PLG-encoding sequence of the invention, and the transferred nucleic acid has no notable rearrangements therein. It is recognized that the transformed plants of the invention may contain the transferred nucleic acid present in low copy numbers (i.e., no more than twelve copies, no more than eight copies, no more than five copies, alternatively, no more than three copies, as a further alternative, fewer than three copies of the nucleic acid per transformed cell).

Transformed plants expressing the aglycosylated PLG polypeptides of the invention (or aglycosylated fragments or variants thereof) can be cultured under suitable conditions for expressing the aglycosylated PLG polypeptide or fragment or variant thereof. The aglycosylated PLG polypeptide or aglycosylated fragment or variant thereof can then be harvested from the plant, the culture medium, or the plant and the culture medium, and then purified using any conventional isolation and purification method known in the art, as described elsewhere herein. The aglycosylated PLG polypeptide or aglycosylated fragment or variant thereof can then be activated to produce a polypeptide having the desired serine protease activity.

Uses of Aglycosylated PLG Polypeptides

The compositions and methods of the present invention are useful in a variety of applications. For example, the various nucleic and amino acid sequences disclosed herein can be used to recombinantly produce stable (i.e., inactive) aglycosylated PLG polypeptides, or stable aglycosylated fragments or variants thereof. The aglycosylated PLG polypeptides (or fragments or variants thereof) can be activated in vitro or in vivo to plasmin using any suitable plasminogen activator known in the art. The plasmin obtained therefrom retains the aglycosylated feature of the aglycosylated PLG polypeptide from which it is derived. The derived plasmin then can be used as a therapeutic for mammalian subjects, including humans, in need of exogenous plasmin, such as subjects having or susceptible to having thrombotic disorders, such as acute peripheral arterial occlusion, hemodialysis graft thrombosis, myocardial infarction, occlusive stroke, deep venous thrombosis, pulmonary embolism, and peripheral arterial diseases. The plasmin also can be used as a therapeutic for subjects having or susceptible to having plasmin deficiency. Alternatively, the aglycosylated PLG (or aglycosylated fragment or variant thereof) can be cleaved to mPLG, which then can be activated in vitro or in vivo to microplasmin and used as a therapeutic. Alternatively still, the aglycosylated PLG (or aglycosylated fragment or variant thereof) or mPLG produced therefrom can be activated in vitro or in vivo and used in combination with other therapeutics, such as plasmin-activated pro-drugs (see, Devy et al. (2004) FASEB J. 18:565-567). Alternatively still, the various nucleic and amino acid sequences are useful as diagnostic tools in research and clinical laboratories, such as in assaying for activators or inhibitors of PLG, mPLG, plasmin, or microplasmin.

In this manner, the present invention also provides plasmin derived from the aglycosylated PLG polypeptides of the invention, and compositions, including pharmaceutical compositions, comprising this plasmin. The plasmin obtained from the aglycosylated PLG polypeptides of the invention is also aglycosylated, and thus lacks at least N-linked glycosylation at the amino acid residue corresponding to the asparagine (Asn) residue at position 289 of the mature human PLG sequence (for example, Asn-289 of SEQ ID NO:4, 16, or 20) from which it is derived, and also may retain the O-linked glycosylation sites occurring at the amino acid residues corresponding to Ser-249 and Thr-346, respectively, of the mature human PLG sequence from which it is derived. In some embodiments, the aglycosylated PLG polypeptides of the invention are produced in duckweed as the host expression system, and the aglycosylated plasmin derived from these aglycosylated PLG polypeptides lack an N-linked glycan at the residue position corresponding to Asn-289 of the mature human PLG sequence and also lack the O-linked glycans at the native O-linked glycosylation sites occurring at the amino acid residues corresponding to Ser-249 and Thr-346, respectively, of the mature human PLG sequence. The aglycosylated plasmin derived from activation of the aglycosylated PLG polypeptides of the invention is able to bind fibrin, even in the absence of the native N-linked glycan at the amino acid residue corresponding to Asn-289 of mature human PLG, as well as in the absence of the O-linked glycans at the native O-linked glycosylation sites occurring at the amino acid residues corresponding to Ser-249 and Thr-346 of the mature human PLG sequence. By “fibrin binding” is intended the aglycosylated plasmin has the ability to bind fibrin. Assays for measuring fibrin binding are well known in the art. See also the fibrin binding assays disclosed in the Experimental section herein below.

The aglycosylated plasmin can be derived from the aglycosylated PLG polypeptides of the invention using any suitable plasminogen activator known in the art. In this manner, the aglycosylated PLG polypeptides of the invention can be activated (i.e., cleaved) to plasmin by using a catalytic concentration of an immobilized or soluble plasminogen activator. Any suitable plasminogen activator known in the art can be used to derive plasmin from the aglycosylated PLG polypeptides. Suitable plasminogen activators include, but are not limited to, streptokinase, tissue plasminogen activator (tPA), and urokinase. Thus, for example, soluble streptokinase, tissue plasminogen activator (tPA), or urokinase will cleave the single-chain aglycosylated PLG polypeptide to produce active plasmin at the Arg560-Val561 peptide bond. The resulting two polypeptide chains of the aglycosylated plasmin are held together by two interchain disulfide bridges. The light chain of 25 kDa carries the catalytic center and is homologous to trypsin and other serine proteases. The heavy chain (60 kDa) consists of five triple-loop kringle structures with highly similar amino acid sequences. Some of these kringles contain so-called lysine-binding sites that are responsible for plasminogen and plasmin interaction with fibrin, alpha2-antiplasmin or other proteins.

Activation of the aglycosylated PLG polypeptides of the invention to obtain aglycosylated plasmin can occur at about 4° C. to about 37° C., and typically takes between about 2 to 24 hours. The aglycosylated PLG polypeptides can be activated in the presence of stabilizers, including but not limited to omega-amino acids and glycerol. Exemplary omega-amino acids can include lysine, epsilon amino caproic acid, tranexamic acid, poly lysine, arginine, and combinations or analogues thereof. Following activation, the aglycosylated plasmin solution can be filtered and further stabilized for several days at neutral pH by the addition of omega-amino acids and sodium chloride and applied to benzamidine-SEPHAROSE for further purification.

The plasminogen activator used in the activation process can be removed using any suitable purification method known to those of skill in the art. For example, aglycosylated plasmin derived from activation of the aglycosylated PLG polypeptide can then be bound to an active plasmin-specific absorbent to substantially remove the plasminogen activator. Since the protein of interest is an active serine protease with trypsin-like specificity, benzamidine may be used as a plasmin-specific absorbent that allows for the capture of the aglycosylated plasmin. Other plasmin-specific absorbents having similar properties as benzamidine may also be used. The benzamidine can be immobilized in a solid support medium. The solid support medium can be a resin or SEPHAROSE. Additionally, hydrophobic interaction may be used to further remove the plasminogen activator.

For example, the cleaved aglycosylated PLG can be contained in a solution of amino acids, sodium chloride, and glycerol, which allows for stability of the solution for several days at neutral pH before it is applied to a benzamidine-SEPHAROSE column equilibrated with about 0.05 M Tris, pH 8.5, 0.5 M NaCl. The column is typically run at 4° C. The front portion of the non-bound peak contains high-molecular weight impurities, with the rest of the nonbound peak being represented by residual non-activated aglycosylated PLG and by inactive autodegradation products of aglycosylated plasmin.

The bound aglycosylated plasmin can then be eluted with an acid buffer or with a substantially neutral omega-amino acid. The aglycosylated plasmin bound to benzamidine-SEPHAROSE can be eluted with an acidic buffer such as glycine buffer. When a substantially neutral pH omega-amino acid is used to elute the bound aglycosylated plasmin the final eluted aglycosylated plasmin solution can be substantially free of degraded aglycosylated plasmin. Typically, the substantially neutral pH amino acid has a pH of value of between about 6.5 to about 8.5. Examples of neutral omega-amino acids include lysine, epsilon amino caproic acid, tranexamic acid, poly lysine, arginine, and analogues and combinations thereof.

The aglycosylated plasmin derived from the aglycosylated PLG polypeptides of the invention can be formulated using any suitable formulation method known to those of skill in the art. In this manner, the present invention also provides pharmaceutical compositions comprising the aglycosylated plasmin derived from the aglycosylated PLG polypeptides of the invention. Such compositions typically include the aglycosylated plasmin and a pharmaceutically acceptable carrier. The phrase “pharmaceutically acceptable carrier” as used herein is a carrier that is conventionally used in the art to facilitate the storage, administration, and/or the healing effect of the therapeutic ingredients. A carrier should be stable (i.e., incapable of reacting with other ingredients in the composition), and it should not produce adverse effects in patients at the dosages and concentrations employed for treatment. Suitable carriers include large stable macromolecules such as albumin, gelatin, collagen, polysaccharide, monosaccharides, polyvinyl-pyrrolidone, polylactic acid, polyglycolic acid, polymeric amino acids, fixed oils, ethyl oleate, liposomes, glucose, sucrose, lactose, mannose, dextrose, dextran, cellulose, sorbitol, polyethylene glycol (PEG), and the like. Slow-release carriers, such as hyaluronic acid, may also be suitable. Other acceptable components in the composition include, but are not limited to, pharmaceutically acceptable agents that modify isotonicity including water, salts, sugars, for example, trehalose, polyols, amino acids, and buffers. Examples of suitable buffers include phosphate, citrate, succinate, acetate, and other organic acids or their salts and salts that modify the tonicity such as sodium chloride, sodium phosphate, sodium sulfate, potassium chloride, and can also include the buffers listed above.

The pharmaceutical composition may additionally comprise a solubilizing agent or solubility enhancer. Examples of such solubility enhancers are described, for example, in Wang et al. (1980) J. Parenteral Drug Assoc. 34:452-462; herein incorporated by reference. Non-limiting examples of solubilizing agents encompassed by the present invention include surfactants (detergents) that have a suitable hydrophobic-hydrophilic balance to solubilize the aglycosylated plasmin. Examples of other solubilizing agents that can be used in compositions of the invention include but are not limited to sodium dodecyl sulfonate, sodium decyl sulfate, sodium tetradecyl sulfate, sodium tridecyl sulfonate, sodium myristate, sodium caproylate, sodium dodecyl N-sarcosinate, and sodium tetradecyl N-sarcosinate. Classic stabilization of pharmaceuticals by surfactants or emulsifiers is described, for example, in Levine et al. (1991) J. Parenteral Sci. Technol. 45(3):160-165.

In addition to those agents disclosed above, other stabilizing agents, such as ethylenediaminetetracetic acid (EDTA) or one of its salts such as disodium EDTA, can be added to further enhance the stability of the pharmaceutical compositions. The EDTA acts as a scavenger of metal ions known to catalyze many oxidation reactions, thus providing an additional stabilizing agent.

Where the composition is used for delivery to a mammal such as a human, the isotonicity of the composition is also a consideration. Thus, in one embodiment, the composition for an injectable solution will provide an isotonicity the same as, or similar to, that of patient serum or body fluids. To achieve isotonicity, a salt, such as sodium chloride, potassium, chloride, or a phosphate buffer, can be added to the solution at an appropriate concentration.

The pH of the pharmaceutical composition comprising the aglycosylated plasmin is also a consideration and can depend upon the pharmaceutically acceptable carrier(s) and other components contained therein. Thus, depending upon the method of formulation, the pharmaceutical compositions comprising the aglycosylated plasmin of the invention can have a pH ranging from about 2.5 to about 11.0, depending upon its intended use. Suitable pH values for the pharmaceutical compositions include, but are not limited to, about 2.5, 3.0, 3.5, 4.0, 4.5, 5.0, 5.5, 6.0, 6.5, 7.0, 7.5, 8.0. 8.5, 9.0, 9.5, 10.0, 10.5, or 11.0, and other such values between about 2.5 to about 11.0.

A thorough discussion of formulation and selection of pharmaceutically acceptable carriers, stabilizers, etc. can be found in Remington's Pharmaceutical Sciences (1990) (18th ed., Mack Pub. Co., Eaton, Pa.), herein incorporated by reference.

In some embodiments, the aglycosylated plasmin of the invention is formulated in a pharmaceutical composition at pH of between about 4.0 and 11.0, including about 4.5 to about 11.0, about 5.0 to about 11.0, about 5.5 to about 11.0 about 6.0 to about 11.0, 6.5 to about 11.0, about 7.0 to about 11.0, about 7.5 to about 11.0, about 8.0 to about 11.0, about 8.5 to about 11.0, about 9.0 to about 11.0, about 10.0 to about 11.0, about 6.0 to about 10.5, about 6.0 to about 10.0, about 6.0 to about 9.5, about 6.0 to about 8.5, about 6.0 to about 8.0, about 6.0 to about 7.0, about 6.5 to about 11.0, about 7.0 to about 10.5, about 7.0 to about 10.0, about 7.0 to about 9.5, about 7.0 to about 9.0, about 7.0 to about 8.5, or about 7.0 to about 8.0. Thus, in some of these embodiments, the aglycosylated plasmin of the invention is formulated in a pharmaceutical composition at a pH of about 4.0, 4.5, 5.0, 5.5, 6.0, 6.5, 7.0, 7.5, 8.0, 8.5, 9.0, 9.5, 10.0, 10.5, or 11.0, and other such values between about pH 4.0 and about pH 11.0.

In some of these embodiments, the aglycosylated plasmin of the invention is formulated in a pharmaceutical composition having a pH of about 4.0 to about 11.0 with glycerol as a stabilizing agent. The glycerol is present in the composition at about 10% to about 50% (volume/volume or v/v). Thus, in some embodiments, the aglycosylated plasmin of the invention is formulated in a pharmaceutical composition having a pH of about 4.0 to about 11.0, including a pH of about 4.0, 4.5, 5.0, 5.5, 6.0, 6.5, 7.0, 7.5, 8.0, 8.5, 9.0, 9.5, 10.0, 10.5, or 11.0, and glycerol at a concentration of about 10%, 15%, 20%, 25%, 30%, 35%, 40%, 45%, or about 50%, and other such values between about 10% and 50% (v/v) glycerol. In some of these embodiments, the aglycosylated plasmin of the invention is formulated in a pharmaceutical composition having a pH of about 7.0 to about 11.0, and about 10-50% (v/v) glycerol, including about pH 7.0, 7.5, 8.0, 8.5, 9.0, 9.5, 10.0, 10.5, or 11.0, and about 10%, 15%, 20%, 25%, 30%, 35%, 40%, 45%, or about 50% (v/v) glycerol. In one such embodiment, the aglycosylated plasmin of the invention is formulated in a pharmaceutical composition having a pH of about 7.0 to about 9.0, and about 10% to about 30% (v/v) glycerol; in yet another embodiment, the aglycosylated plasmin of the invention is formulated in a pharmaceutical composition having a pH of about 7.0 to about 9.0, including pH 7.0, 7.5, 8.0, 8.5, or 9.0, and about 20%, 25%, or 30% (v/v) glycerol.

Formulating aglycosylated plasmin at a pH of about 4.0 to about 11.0, particularly pH 7.0 to about 11.0, with glycerol as a stabilizing agent at about 10% to about 50% (volume/volume or v/v) provides for stabilization of the aglycosylated plasmin. Thus, the present invention also provides a method for stabilizing aglycosylated plasmin by formulating it at a pH of about 4.0 to about 11.0 with glycerol at a concentration of about 10% to about 50%.

The aglycosylated plasmin of the invention finds use in any therapeutic application suitable for the glycosylated form of plasmin. Thus, compositions comprising the aglycosylated plasmin, including the pharmaceutical compositions described herein above, find use in treating thrombotic disorders, such as acute peripheral arterial occlusion, hemodialysis graft thrombosis, myocardial infarction, occlusive stroke, deep venous thrombosis, pulmonary embolism, and peripheral arterial diseases. In this manner, the present invention provides a method of treating a mammalian subject, including a human, for a thrombotic disorder by administering a therapeutically effective amount of an aglycosylated plasmin derived from the aglycosylated PLG polypeptides of the invention. By “therapeutically effective amount” is intended an amount sufficient to lyse a thrombus and restore blood flow. A therapeutically effective amount can be determined based on severity of clot, patient history, including patient age, sex, weight, etc.

In this manner, the therapeutic methods of the invention find use in treating a thrombus or thrombotic occlusion. By “thrombus” is intended a thrombus in a blood vessel or device contacting blood (e.g. catheter devices or shunts). A thrombus may comprise fibrin and may further comprise platelets, erythrocytes, lymphocytes, lipid or any combination thereof. A “thrombus” may be, but is not limited to, an annular thrombus, ball thrombus, hyaline thrombus, mural thrombus, stratified thrombus or white thrombus. By “thrombotic occlusion” is intended a partial or total blockage of a vessel due to the formation of a thrombotic clot, wherein the thrombus comprises at least fibrin. The vascular vessel occluded may be, but is not limited to, a vein, artery, venule, arteriole, capillary, vascular bed or the heart and may be within any vascularized organ or tissue of the subject in need of treatment. The thrombotic occlusion may also be of a catheter or other implant including, but not limited to, prosthetic vessels and grafts of synthetic, human or animal origin and effectively blocked by an occlusion comprising fibrin. The term “catheter device” is intended to mean any catheter or tube-like device that may enter the body, and includes but is not limited to, an arterial catheter, cardiac catheter, central catheter, central venous catheter, intravenous catheter, pheripherally inserted central catheter, pulmonary arter catheter or tunneled central venous cather and arterio-venal shunts.

In some embodiments, a composition comprising the aglycosylated plasmin of the invention, including the pharmaceutical compositions described herein above, is administered by any method that will deliver the aglycosylated plasmin as a bolus or as a prolonged infusion directly into a thrombus, or to a site a short distance proximal to the thrombus whereupon the aglycosylated plasmin composition can rapidly encounter the thrombus. By minimizing the distance from the catheter to the thrombus, the aglycosylated plasmin composition's exposure to serum inhibitors is reduced. Catheter delivery to a thrombus allows precision in placing the aglycosylated plasmin composition, especially within the thrombus.

The following examples are offered by way of illustration and not by way of limitation.

### EXPERIMENTAL

### Example 1

### Improved Recovery of PLG from Duckweed by Use of a Human, Aglycosylated PLG

Recombinant human plasminogen (PLG) has previously been produced in Lemna, a member of the duckweed family (see U.S. Patent Application Publication No. 20050262592, the contents of which are herein incorporated by reference in their entirety). The collected PLG product can be fully activated to an aglycosylated human plasmin for use in a variety of clinical indications, as noted elsewhere herein. It has been discovered that improved recovery of stable PLG from the duckweed expression system is possible when the PLG protein structure is altered such that N-linked glycosylation at Asparagine (Asn)-289 of the mature human PLG sequence is prevented. In this manner, the coding sequence for human PLG was modified to include a codon for an aspartic acid residue in place of Asn-289 to prevent attachment of the typical plant N-linked glycan at this position of the mature PLG protein (see N289D protein sequence shown in SEQ ID NO:6, encoded by codon-optimized sequence shown in SEQ ID NO:5). Expression and recovery of mature human PLG was significantly improved as a result of this modification.

Methods:

Plasmid Construction.

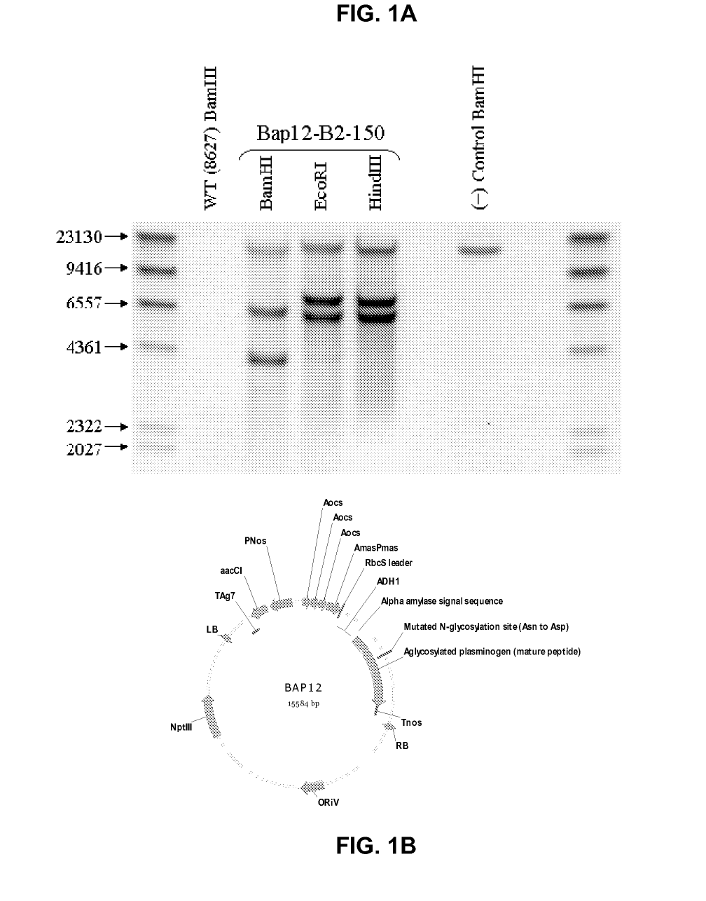

BAP12 was created by joining the rice alpha-amylase signal peptide coding sequence (see nucleotides 1-93 of GenBank Accession No. M24286) to the 5′ end of the mature human PLG coding region (see SEQ ID NO:3). Both the signal peptide coding sequence and PLG coding sequence were optimized by replacing the native sequences with frequently used Lemna codons. Additionally, the single native N-glycosylation site at amino acid position 289 of the mature human PLG protein was mutated from Asn to Asp (see SEQ ID NO:6 for N289D PLG protein sequence). The codon-optimized rice alpha-amylase signal peptide coding sequence is set forth in SEQ ID NO:7. The codon-optimized sequence encoding the N289D variant of mature human PLG is set forth in SEQ ID NO:5. The binary vector used for BAP12 was a modification of pBMSP3 (obtained from Dr. Stan Gelvin, Purdue University) in which the 5′-mas leader was replaced with the leader from the rubisco carboxylase small subunit 5B gene derived from L. gibba (sequence shown in SEQ ID NO:10). This vector includes the maize ADH1 intron (sequence shown in SEQ ID NO:9) positioned immediately upstream of the alpha-amylase signal/N289D PLG fusion polypeptide. The sequence was confirmed by DNA sequencing. See FIG. 1B for the plasmid map. The complete codon-optimized coding sequence for the alpha-amylase signal/N289D PLG fusion polypeptide is shown in SEQ ID NO:11, and the encoded fusion polypeptide is shown in SEQ ID NO:12.

Transformation and Plant Regeneration.

Approximately 20 to 50 ng of BAP12 plasmid DNA was electroporated into A. tumefaciens C58Z707 and selected on kanamycin (50 mg/L) in LB media. A. tumefaciens containing the binary vector was heavily streaked onto a LB plate (50 mg/L Kanamycin, 100 μM acetosyringone) and incubated for two days at 28° C. The A. tumefaciens was then suspended in bacterial resuspension medium (MS basal medium, 0.6 M mannitol, 100 μM acetosyringone, pH 5.6) at an approximate OD₅₉₅ of 1.0 and incubated for at least 1 hour at room temperature. Green, healthy, rapidly growing nodules from Lemna minor 8627 were then submerged in the bacterial suspension for three to five minutes and placed on co-cultivation medium (MS basal medium, 30 g/L sucrose, 1 μM 2,4-dichlrophenoxyacetic acid, 2 μM 6-benzylaminopurine, 0.4% Bacto-agar, 0.15% Gelrite, and 100 μM acetosyringone, pH 5.6) and incubated at 27° C. After two days, nodule pieces were then transferred to selection/regeneration medium containing ½ strength Shenck & Hildebrandt (SH) major and minor elements with vitamins, 0.4% Bacto-agar, 1.5% Gelrite, 200 mg/L geneticin, and 500 mg/L cefotaxime. The plates were placed in a 27° C. growth chamber in direct and constant light (approximately 40 mmol/m²/s²) and transferred weekly to fresh medium with antibiotics. The single fronds that emerged were then placed in 30 ml of liquid SH major and minor elements with vitamins, 1% sucrose, pH 5.6 containing cefotaxime (500 mg/L).

Transgenic Line Maintenance and Archiving

Once individual clonal lines were propagated in liquid SH culture with 1% sucrose, lines were maintained in duplicate agar backup plates containing ½ SH components (with 1% sucrose) containing cefotaxime at 27° C. Plates were wrapped in colored plastic and maintained under low light in a growth room. A new set of backup plates was made every six weeks. Once transgenic lines were screened for recombinant protein expression, superior lines were selected for archival. Three independent sets of archival lines were created and maintained on SH agar plates with 1% agar (with and without 4 μg/ml Genetecin) at 23° C. in a Percival growth chamber under continuous 40 μE/m²/second light and on slants of Hoagland's medium with 1% agar at 10° C. in a Percival growth chamber under continuous 10 μE/m²/second light. Archival lines maintained on SH media were transferred to fresh plates every two months while lines maintained on Hoagland's media were transferred to fresh slants every six months.

Primary Transgene Screening

Screening was accomplished in vented research vessels (known as IVs). IVs were grown under continuous light of ˜170-200 μE/m²/second at a temperature ˜24° C. IVs are 125 ml PET vessels. The open orifice is covered by Milliwrap® (Millipore). Fronds from the harvesting vessel are placed in a conditioning IV with 50 ml of Media 1.2 for one week. Media 1.2 is a photosynthetic version of SH medium with 10 mM sodium PIPES ˜pH 6.0 to 6.1 prepared by Hyclone. After one week, 6 three-frond clusters were placed in a screening IV and grown for two weeks. At two weeks, the fronds were harvested and tissue samples (250 mg) were placed in screw cap tubes and stored at −80° C. Screening was accomplished by plasmin activity analysis using the Chromogenix Coamatic Plasminogen Kit (Diapharma catalog #K822452).

Secondary Screening

Secondary screening was performed in the same manner as primary screening. Secondary screening of the lines shown to be producing PLG was accomplished by obtaining material for the lines in question from the backup plate and placed in 50 ml of Media 1.2 in a preconditioning vessel for one week, followed by picking 6 three-frond clusters into 50 ml of Media 1.2 in an IV and grown for two weeks. At two weeks, the tissue was harvested and samples (250 mg) were placed in screw cap tubes and stored at −80° C. Screening was accomplished using the Chromogenix Coamatic Plasminogen Kit.

DNA Extraction and Southern Blot Analysis

The procedure for DNA extraction was performed as described in Dellaporta et al. (1983) MNL 57:26-29, and Southern blot hybridization was performed according to manufacturer's recommendations (Vacuum Blotter Instruction Manual, Bio-Rad Laboratories, Hercules, Calif., USA). Genomic DNA from BAP12-B2-150 was digested with restriction enzymes BamHI, EcoRI and HindIII (New England Biolabs). Southern blot was probed with a DIG labeled fragment of the PLG gene.

Sequencing of BAP12-B2-150 cDNA. cDNA was synthesized using the SuperScriptIII cDNA synthesis kit (Invitrogen) according to the manufacturer's recommendations. PCR was performed on the cDNA using DNA primers (Biolex 502 and Biolex 503) specific to 5′UTR and 3′UTR of PLG gene within the BAP12 cassette. PCR products were cloned into the pCRII-Blunt-TOPO vector (Invitrogen). Five independent clones were chosen for sequence analysis. DNA sequencing was carried out at GeneWiz Inc. DNA Sequencing Facility. PLG cDNA PCR products were completely sequenced with DNA primers specific to the TOPO cloning vector and PLG gene sequence.

Phytoplasma and Bioburden Testing of the Line BAP12-B2-150

Fronds were taken from the BAP12-B2-150 Research Archive slant and used to inoculate an IV containing 50 ml of Media 1.2. The liquid culture was sub-cultured every ten to fourteen days, with 6 three-frond clusters being used to inoculate a new IV containing 50 ml of Media 1.2, establishing a continuous liquid culture. From this continuous liquid culture, the bioburden was determined prior to culture harvest, by placing some fronds and media on a tryptic soy agar plate and placing the plate at 25° C. for 7 days.

At harvest, tissue was harvested by removing the media with a filter sterilization unit, and the tissue was placed in roughly equal amounts in two 50 ml sterile conical tubes. The tissue was snap frozen in liquid nitrogen and stored at −80° C. The tissue was sent on dry ice to the California Seed and Plant Lab (Elverta, Calif.) for PCR determination of the presence of Phytoplasma DNA sequences in the frozen tissue.

BAP12-B2-150 Slant Stability for the Production of PLG in the Tissue

BAP12-B2-150 agar slants were made and then stored at 9° C. at a light level of 1-5 μmols/m²/second of light. The slants will be sampled at 3, 6, 9, and 12 months and tested for stability of the production of plasminogen protein in the tissue. At each time point, four independent IVs, containing 50 ml of Media 1.2, will be inoculated with 1 three-frond cluster from a slant. The IVs will be grown at 25° C. and at 250 μmols/m²/second of continuous light. The tissue from the IVs will be harvested after sixteen to eighteen days growth. At harvest, the tissue samples (250 mg) are placed in screw cap lysing tubes, snap frozen in liquid nitrogen, and stored at −80° C. Samples are analyzed by using the COAMATIC® Plasminogen Assay Kit (82-2452-63) (Diapharma, Inc.)

Growth of Line BAP12-B2-150 and Total PLG Yield in the Tissue from IVs Inoculated from a 9° C. Slant.

BAP12-B2-150 slants were stored at 9° C. at a low light level of 1 to 5 μmmol/m²/second. Each slant contained 18 to 20 three-frond clusters. The slants were used to inoculate 50 ml of Media 1.2 (pH 6.1) in IVs on the dates shown below in Table 4.

A control antibody line, CNTD02-200, that produces an IgG was used as a negative control. Each IV was inoculated with 3 three-frond clusters of BAP12-B2-150 from each slant as shown in Table 4 below. The IVs were grown at 25° C. at a light level of 250 μmol/m²/second. The IVs were harvested according to the dates shown in Table 4 and the fresh weight of fronds determined at harvest. From each harvested IV, tissue samples (250 mg) were placed in green capped lysing tubes to determine the amount of total PLG in the tissue sample using the COAMATIC® Plasminogen Assay Kit.

TABLE 4
Experimental design of growth and presence of plasminogen protein from liquid
cultures started from BAP12-B2-150 Q agar slants.

| | | Source of | | | | | |
|---|---|---|---|---|---|---|---|
| | Date agar | inoculum | | Date | Day | Harvest | Days of |
| Line | slant made | slant # | Replicate | started | started | date | growth |
| BAP12-B2- | Oct. 20, 2008 | 49 | 1 | Nov. 24, 2008 | 1 | Dec. 10, 2008 | 16 |
| 150 | | | | | | | |
| BAP12-B2- | Oct. 20, 2008 | 49 | 2 | Nov. 24, 2008 | 1 | Dec. 10, 2008 | 16 |
| 150 | | | | | | | |
| BAP12-B2- | Oct. 20, 2008 | 49 | 3 | Nov. 24, 2008 | 1 | Dec. 10, 2008 | 16 |
| 150 | | | | | | | |
| BAP12-B2- | Oct. 20, 2008 | 49 | | Nov. 24, 2008 | 1 | Dec. 12, 2008 | 18 |
| 150 | | | | | | | |
| BAP12-B2- | Oct. 20, 2008 | 49 | | Nov. 25, 2008 | 2 | Dec. 11, 2008 | 16 |
| 150 | | | | | | | |
| BAP12-B2- | Oct. 20, 2008 | 46 | 1 | Nov. 24, 2008 | 1 | Dec. 10, 2008 | 16 |
| 150 | | | | | | | |
| BAP12-B2- | Oct. 20, 2008 | 46 | 2 | Nov. 24, 2008 | 1 | Dec. 10, 2008 | 16 |
| 150 | | | | | | | |
| BAP12-B2- | Oct. 20, 2008 | 46 | 3 | Nov. 24, 2008 | 1 | Dec. 10, 2008 | 16 |
| 150 | | | | | | | |
| BAP12-B2- | Oct. 20, 2008 | 46 | | Nov. 24, 2008 | 1 | Dec. 12, 2008 | 18 |
| 150 | | | | | | | |
| BAP12-B2- | Oct. 20, 2008 | 46 | | Nov. 25, 2008 | 2 | Dec. 11, 2008 | 16 |
| 150 | | | | | | | |
| CNTD02-B1- | Research | | 1 | Nov. 24, 2008 | 1 | Dec. 10, 2008 | 16 |
| 200 | Archive | | | | | | |
| CNTD02-B1- | slant | | 2 | Nov. 24, 2008 | 1 | Dec. 10, 2008 | 16 |
| 200 | | | | | | | |
| CNTD02-B1- | | | 3 | Nov. 24, 2008 | 1 | Dec. 10, 2008 | 16 |
| 200 | | | | | | | |
| BAP12-B2- | Oct. 20, 2008 | 52 | 1 | Nov. 25, 2008 | 2 | Dec. 11, 2008 | 16 |
| 150 | | | | | | | |
| BAP12-B2- | Oct. 20, 2008 | 52 | 2 | Nov. 25, 2008 | 2 | Dec. 11, 2008 | 16 |
| 150 | | | | | | | |
| BAP12-B2- | Oct. 20, 2008 | 52 | 3 | Nov. 25, 2008 | 2 | Dec. 11, 2008 | 16 |
| 150 | | | | | | | |
| BAP12-B2- | Oct. 20, 2008 | 52 | | Nov. 24, 2008 | 2 | Dec. 12, 2008 | 18 |
| 150 | | | | | | | |
| BAP12-B2- | Oct. 20, 2008 | 47 | 1 | Nov. 26, 2008 | 3 | Dec. 12, 2008 | 16 |
| 150 | | | | | | | |
| BAP12-B2- | Oct. 20, 2008 | 47 | 2 | Nov. 26, 2008 | 3 | Dec. 12, 2008 | 16 |
| 150 | | | | | | | |
| BAP12-B2- | Oct. 20, 2008 | 47 | 3 | Nov. 26, 2008 | 3 | Dec. 12, 2008 | 16 |
| 150 | | | | | | | |
| BAP12-B2- | Oct. 20, 2008 | 47 | | Nov. 24, 2008 | 3 | Dec. 12, 2008 | 18 |
| 150 | | | | | | | |

Brief Description of BAP12 Plasmin Purification Using GLP Process (BAP12-B2-150).

Lemna-derived PLG was extracted from BAP12-B2-150 using a Waring Blender and chilled extraction buffer—50 mM Tris, 300 mM NaCl, 10 mM EDTA, pH 9.0—at a biomass to buffer ratio of 1:4. The extract was homogenized and acid precipitated to pH 2.5 using concentrated HCL. The precipitate was removed by centrifugation at 16,000×g, 4 C, for 30 minutes. The supernatant was retitrated to pH 9.0 with concentrated NaOH, filtered and then loaded on a lysine-Sepharose Column (GE Healthcare) equilibrated with 50 mM Tris, 300 mM NaCl, 10 mM EDTA, 10% glycerol, pH 9.0. The column was washed with equilibration buffer followed by an intermediate wash with 25 mM Tris, 10 mm NaCl, pH 9.0 to lower conductivity. The column was eluted with 50 mM Tris, 50 mM EACA, pH 9.0. Lemna-derived PLG was concentrated using the anion exchange resin Poros 50 HQ in an effort to put the eluate in a suitable buffer for streptokinase (SK) activation. PLG was activated using streptokinase (SK). Briefly, glycerol was added to the PLG solution to a final concentration of 20%. The activation was started by adding 250 IU SK/mg PLG. The mixture was incubated at room temperature and plasmin generation was monitored over time using the activity assay. After activation, the generated plasmin was purified using Benzamidine sepharose 6 FF. Briefly, the mixture was applied to the column equilibrated in 50 mM Tris, 500 mM NaCl, 10% glycerol, pH 8. Plasmin was specifically bound to the column and unretained components were washed off with equilibration buffer and an additional wash of 5 mM Tris, 10% glycerol, pH 8. Plasmin was eluted with a step-gradient to 50 mM sodium acetate, 10% glycerol, pH 4.

Benzamidine eluate material was further purified with the cation-exchange resin CM FF to remove remaining SK and any 54 kD plasmin fragment from the preparation. Briefly, the benzamidine eluate was loaded directly onto the CM FF column followed by a wash to baseline with equilibration buffer and an intermediate wash with 50 mM sodium acetate, 400 mM NaCl, 10% glycerol, pH 4. The column was eluted with a step-gradient to 50 mM sodium acetate, 550 mM NaCl, 10% glycerol, pH 4. The CM eluate was formulated in 5 mM sodium acetate-glacial acetic acid pH 3.7 containing 10% glycerol at 2.5 mg/ml.

SDS-PAGE and Western Blot Analysis.

Crude extract and partially purified PLG were analyzed by western blot and SDS-PAGE using 4%-20% Tris-glycine gels (Invitrogen) under either reducing or nonreducing conditions for initial characterization of the PLG. SDS-PAGE gels were stained with the Colloidal Blue Stain Kit (Invitrogen). Purified plasmin samples were analyzed by SDS-PAGE also using 4%-20% Tris-glycine gels, and stained with the Colloidal Blue Stain Kit.

PLG and Plasmin Activity Assay.

The PLG/plasmin concentration in solution is determined at ambient temperature using the COAMATIC® Plasminogen Assay Kit (82-2452-63) (Diapharma, Inc.) according to the manufacturer's instructions. For PLG, samples and standards were first activated by SK. No activation is required for plasmin samples prior to analysis. The concentration of PLG or plasmin in the sample is proportional to the initial rate of substrate (S-2403) hydrolysis. Hydrolysis of S-2403 produces a yellow color which is monitored by absorbance at OD₄₀₅ nm. The plasmin concentration in the sample is calculated using a standard curve that was generated from a serial dilution of standard human plasmin ranging from 15.0 μg/ml to 0.156 μg/ml.

Inhibition of Plasmin by α₂-AP (SDS PAGE).

α₂-AP is an inhibitor of the serine protease inhibitor (serpin) family Inhibition of serine proteases by serpins is based on the irreversible complex formation between the protease and the serpin. In order to demonstrate complex formation between plasmin and α₂-AP, plasmin samples, either derived from line BAP12-B2-150 or the human reference plasmin were incubated with a 1.1-fold molar excess of α₂-AP (10 minutes, TBST) and run on SDS-PAGE gels. Briefly, plasmin and α₂-AP were mixed at a molar ratio of 1:1.1 in TBS (Tris 50 mM pH 7.5 and 150 mM NaCl). The ratio was derived from the specific activity of the inhibitor provided by the manufacturer. After 30 minutes at room temperature, 1 volume of 2×SDS sample buffer was added and the samples were analyzed on SDS gels.

Inhibition of Plasmin by α₂-AP (Chromogenic Activity).

Inactivation of human reference plasmin and BAP12-B2-150 plasmin activity was measured after incubation of the respective species with increasing concentration of α₂-AP using the plasmin activity assay to measure residual plasmin activity. Equal volumes (50 μl) of α₂-AP and plasmin were incubated for 30 minutes at ambient temperature before 50 μl S-2403 was added and the plate was read at 405 nm to obtain initial rates. The samples were analyzed on 4%-20% gradient gels, under non-reducing conditions, and were stained using Coomassie blue. The protein bands were identified on the right side of the gel. 2.5 μg plasmin by activity analysis were loaded in each lane.

Fibrinolysis.

Fibrinolysis assays were performed in micro-titer plates (Microtest™ 96-well, 370 μL clear plate UV-VIS transparent film bottom; BD Falcon) containing fibrin formed in the following manner: 100 μl/well of purified fibrinogen (Haematologic Tech Inc.) was prepared in HEPES 20 mM, pH 7.2, NaCl 150 mM and 2 mM CaCl₂ at 1 mg/ml. The clot formation was monitored at 340 nm, in kinetic mode, for 2 hours and reading every 30 seconds and with the auto mix on for 5 seconds. Clot formation was initiated by adding 10 μA α-thrombin (Haematologic Tech Inc. Cat# HCT-0020) at 2 international units/ml, and the recording at 340 nm was immediately started. Clot formation was followed at 340 nm until a plateau was reached.

For analysis of fibrinolysis activity, plasmin solutions at 500, 250, 125, and 62 nM were prepared in HEPES 20 mM, pH 7.2, NaCl 150 mM, containing 10% glycerol. The template was set up as previously described for fibrin clot formation except that the recording time was extended to three hours and the auto mix was switched off. Fibrinolysis was initiated by layering 100 μA of the appropriate plasmin sample on top of the clots. Clot lysis was monitored as the decrease of turbidity at 340 nm over time.

Peptide Mapping.

The primary sequence identity of plasmin was derived from a lysylendopeptidase (LysC) digestion. Plasmin (1 mg/ml) was denatured in 3 M urea, and disulfide bonds were reduced with 8 mM dithiothreitol during a 4-hour, room temperature digestion with LysC. Digests were chromatographed for 65-minutes on a 2%-36% acetonitrile gradient on a 2.1×25 mm Supelco Discovery BIO Wide Pore C18 column. Eluting peaks were analyzed by MS (Q-T of), obtaining peptide sequence data from alternating high and low fragmentation energies.

Plasmin Analysis using RP Chromatography.

The reverse-phase assay utilizes chromatography on a C18 RP column to separate multiple forms of plasmin in the final formulated preparation. Using this assay, plasmin purified from both line BAP01-B2-230 and BAP12-B2-150 was separated into five peaks. The peaks were collected and analyzed by SDS-PAGE and plasmin activity assay.

Stability Analysis.

(1) Freeze-thaw stability: BAP12-B2-150 and human reference plasmin were tested for the effects of three freeze-thaw cycles on the stability of the samples. Solutions of human reference plasmin at 2.5 mg/ml in 10 mM citric acid and 30 mM NaCl and BAP12-B1-150 plasmin at 2.5 mg/ml in H₂O pH 3.7 and 10% glycerol were subjected to the following treatment: 1. Thaw at ambient temperature for 60 minutes; 2. Frozen for two hours at −80° C.; 3. Thaw for 60 minutes at ambient temperature; 4. Frozen for two hours at −80° C.; 5. Thaw for 60 minutes at ambient temperature; and 6. Frozen overnight at −80° C. Samples were taken at steps 1 and 6 and the plasmin concentration measured by activity analysis was determined. Samples were also analyzed by SEC to determine the presence of aggregate or degradation products. Similar analytical tests were performed after thawing the sample from step 6.

(2) Stability of BAP12-B1-150 plasmin at ambient temperature and at 4° C.: The protocol for the stability characterization of BAP12-B1-150 at ambient temperature (i.e., 25° C.) and at 4° C. is the same as used for the GLP production runs and as a plasmin release assay. As a result, these data can be directly compared to stability data of plasmin derived from the line BAP01-B2-230. Two vials of BAP12-B1-150 plasmin at 2.5 mg/ml in H₂O pH 3.7 (glacial acetic acid) containing 10% glycerol were thawed. One vial was left on the bench top at room temperature, the second at 4° C. At 0, 2, 4, and 24 hours, samples were taken for plasmin activity concentration measurement using activity analysis and for SEC analysis to determine the presence of aggregate or degradation products. For the plasmin sample stored at 4° C., an additional sample was analyzed after one week.

Results:

Southern Blot Analysis.

A Southern Blot was developed to characterize the t-DNA insertion pattern for BAP12-B2-150. A panel of restriction endonucleases was tested in order to identify the three enzymes used in this analysis. The banding pattern resulting from BamHI, EcoRI, and HindIII digestion presents the distinct migration pattern shown in FIG. 1A.

Transgene Sequence Analysis.

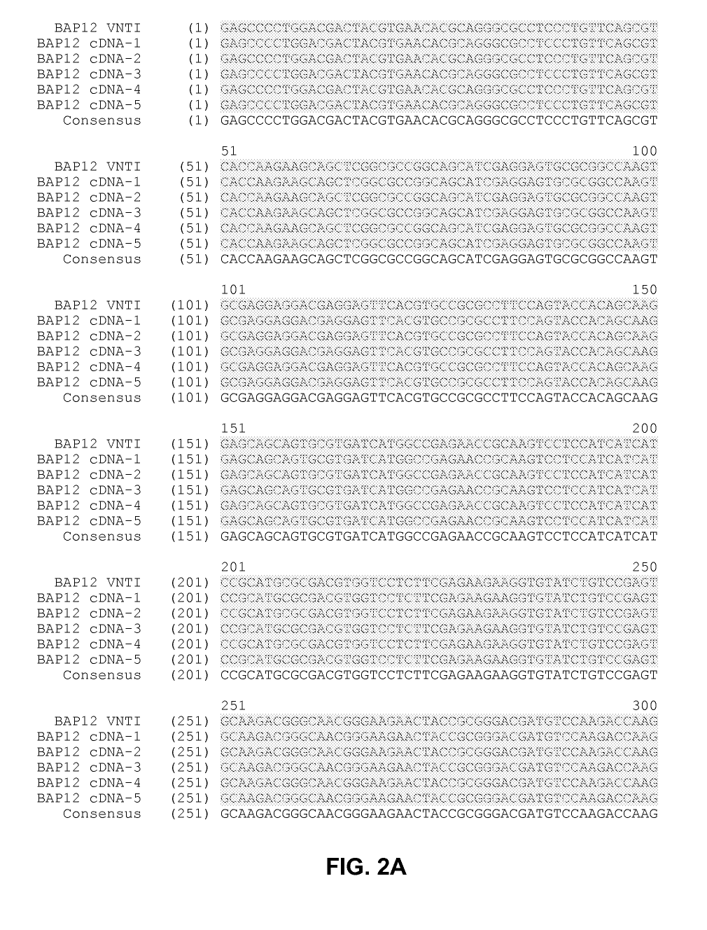

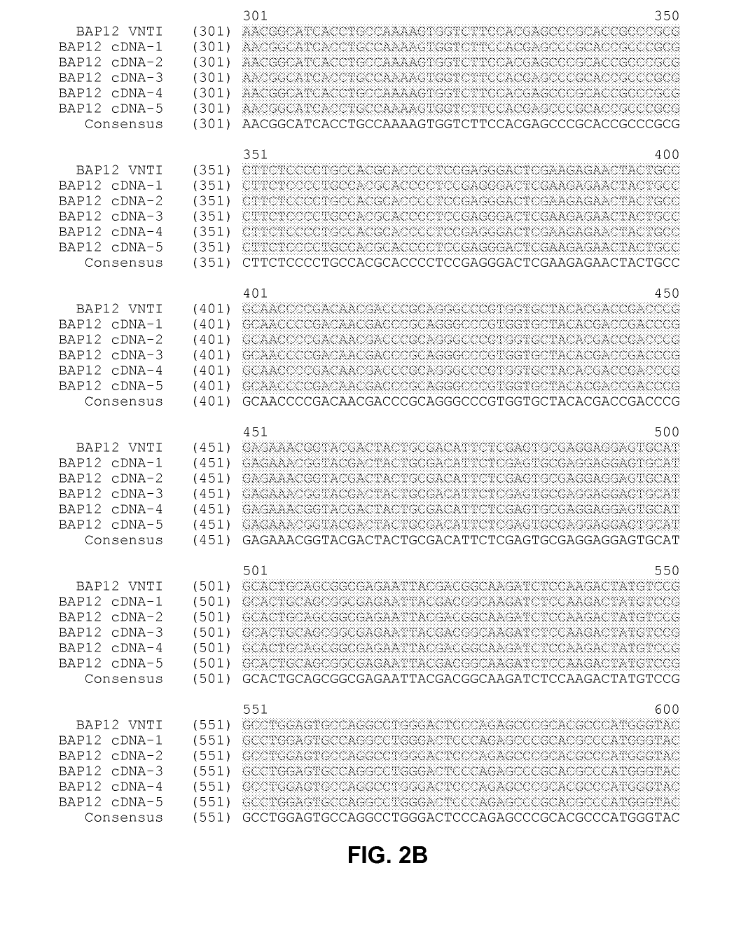

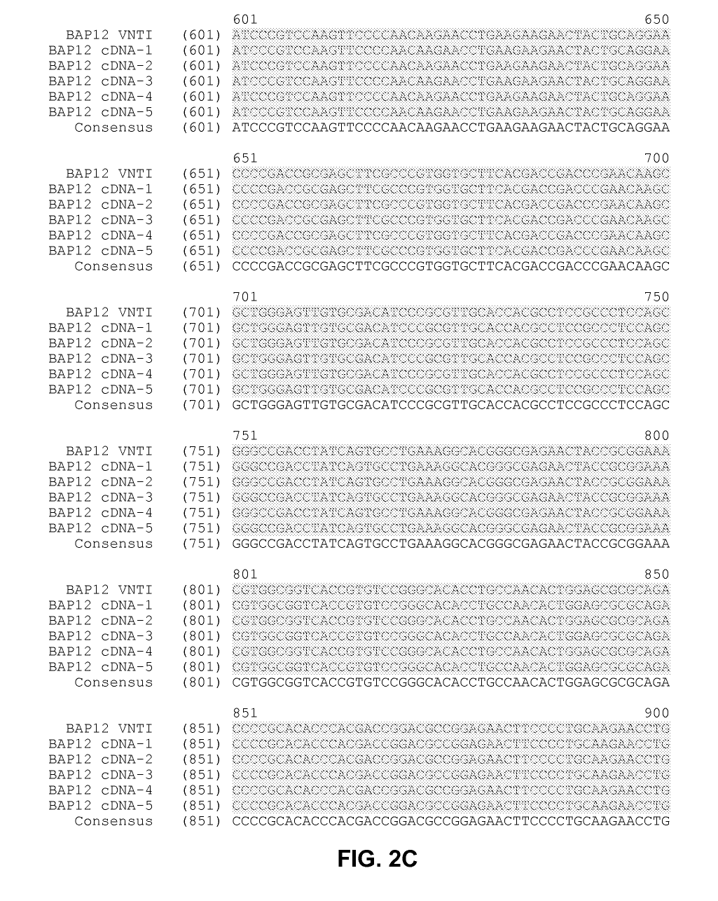

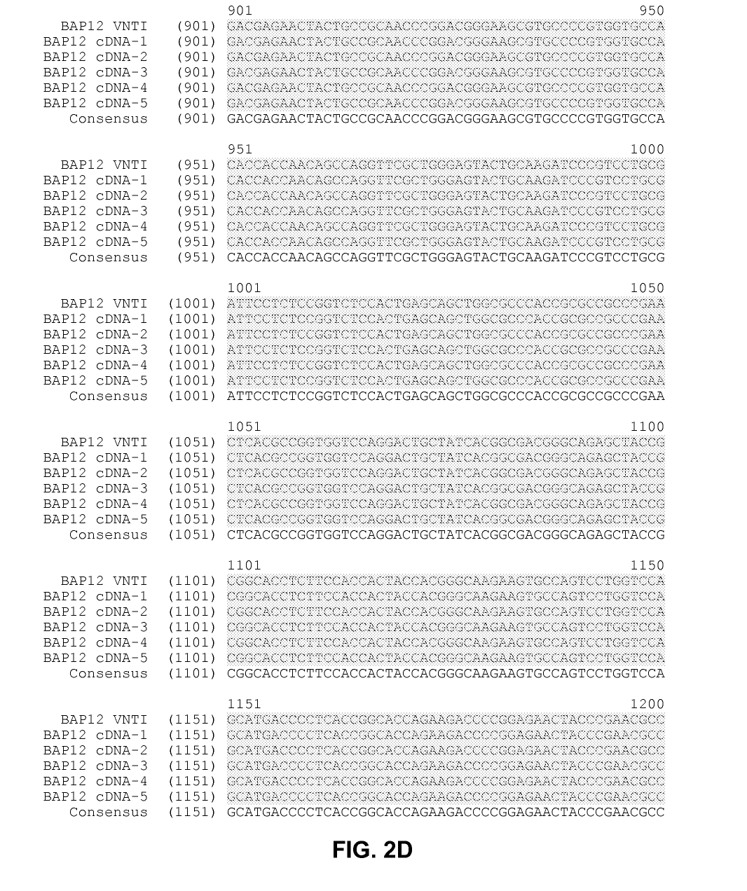

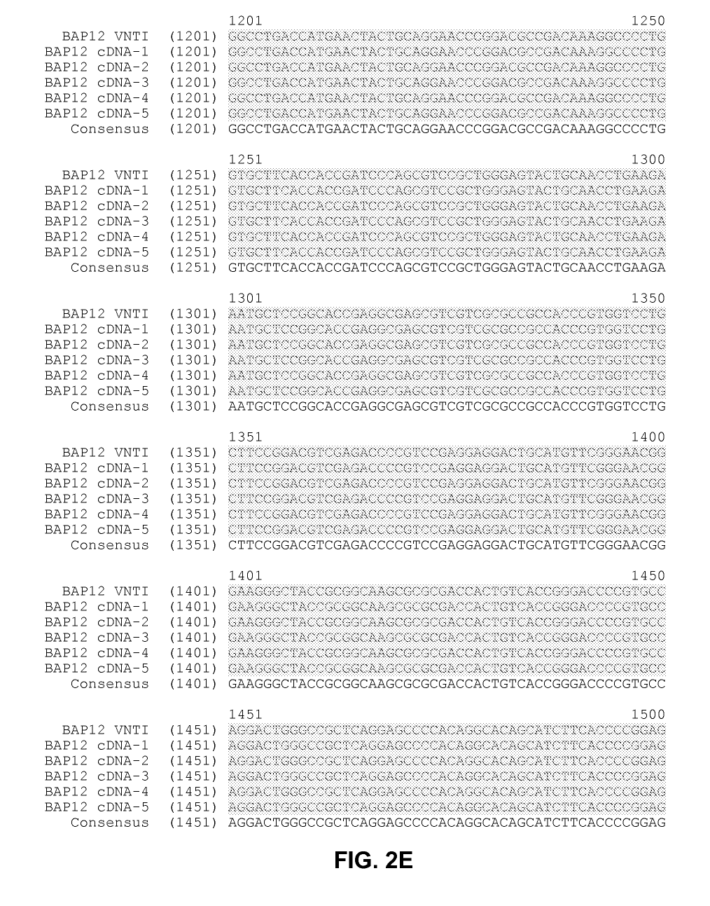

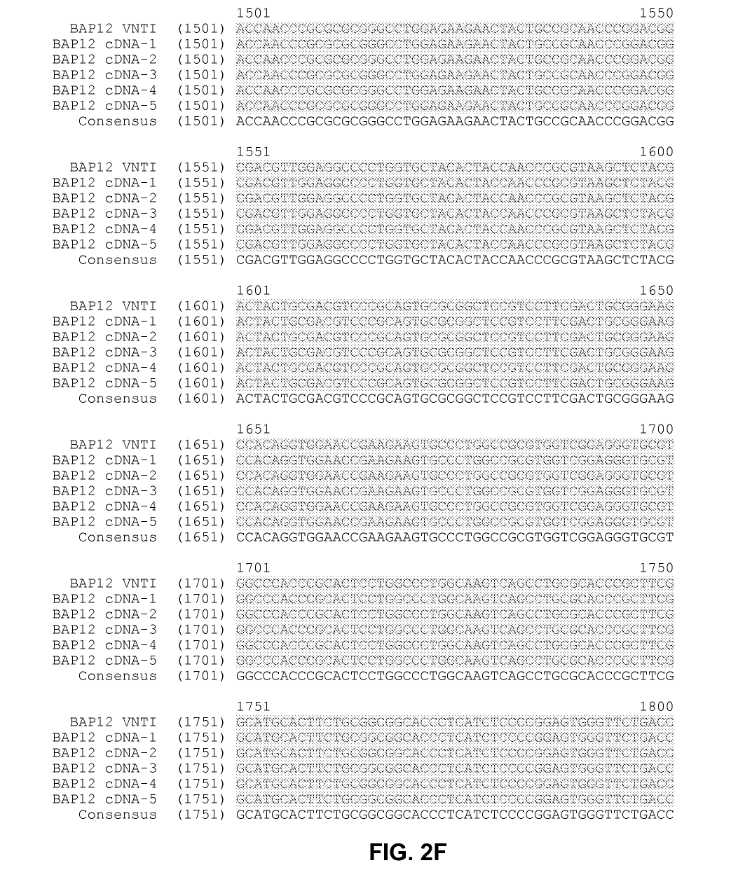

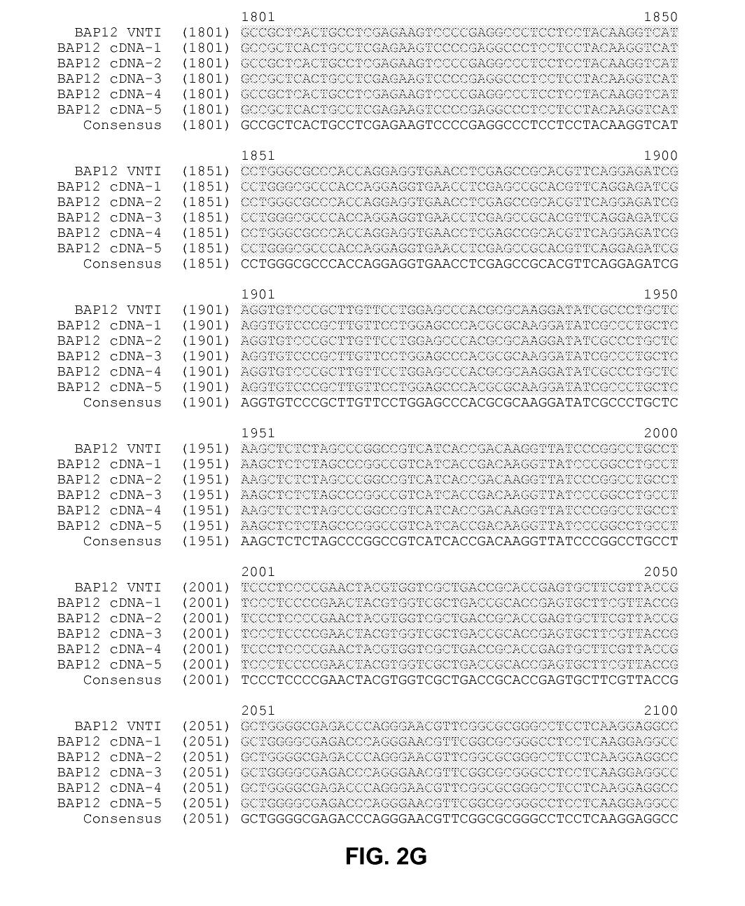

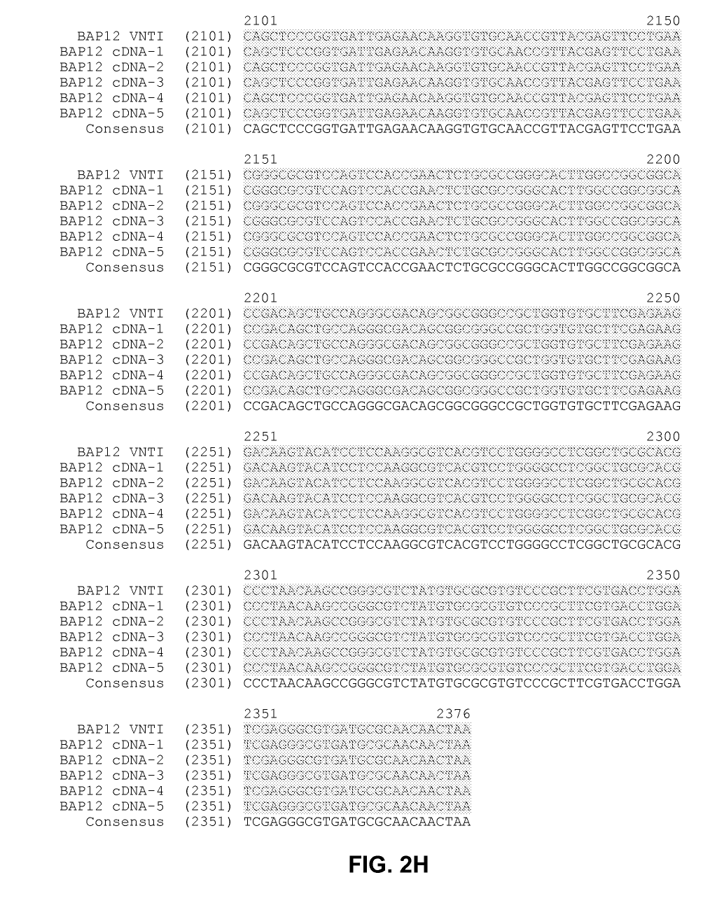

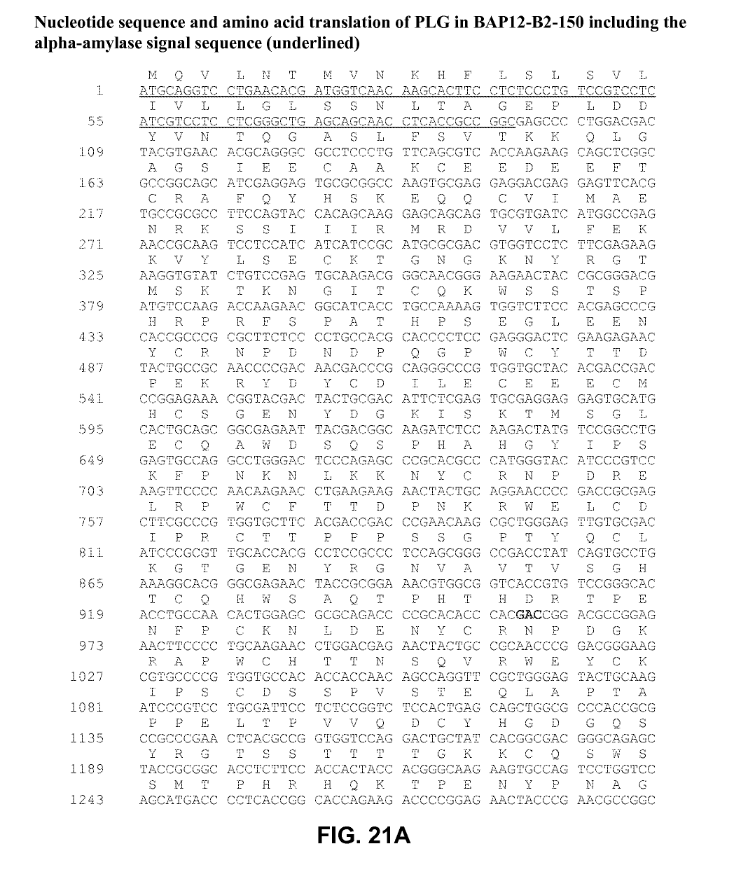

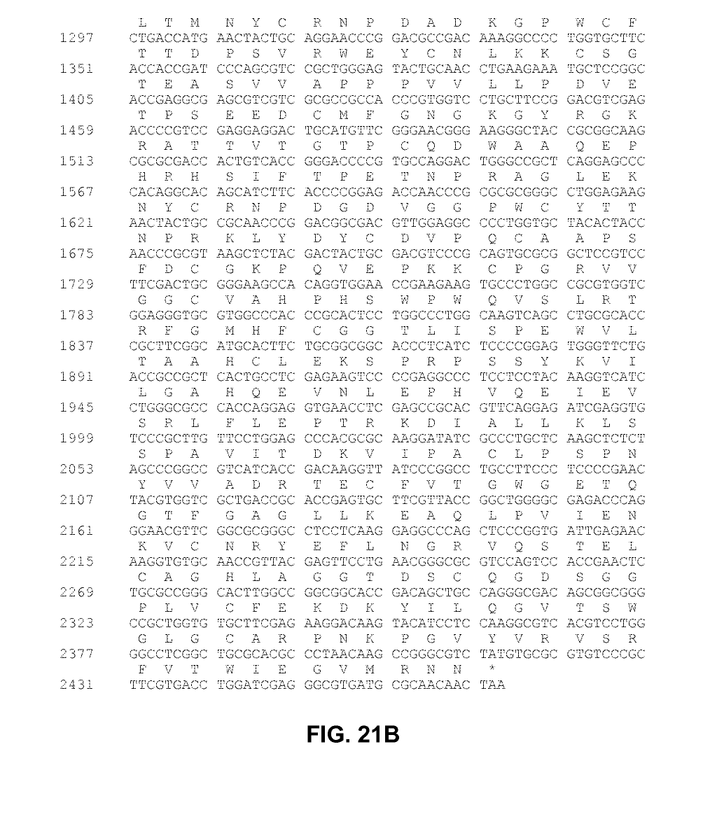

The DNA sequence alignment of the five cDNA clones from BAP12 is shown in FIGS. 2A-2H. The expected sequence of the Lemna-derived mature PLG having the N289D substitution is denoted by “BAP12 VNTI” (Vector NTI). The consensus of all of the sequences is given on the last line of the alignment. All cDNA sequences generated from BAP12-B2-150 are 100% identical to the PLG reference sequence. The nucleotide sequence and translated amino acid sequence for BAP12-B2-150 PLG (including the alpha-amylase signal peptide) are shown in FIGS. 21A and 21B.

BAP12-B2-150 Line History and Production of BAP12-B2-150 Q Agar Slants.

The line history of BAP12-B2-150 is shown below in Table 5. The BAP12-B2-150 agar Q-slants were made using the continuous liquid culture as a source of inoculum. Their health and genetic stability will be monitored every three months.

TABLE 5
Line history of BAP12-B2-150.

| Line History: BAP12-B2-150 | Date |
|---|---|
| Nodule Transformation | Aug. 1, 2007 |
| Line Regeneration | Oct. 2, 2007 |
| Initial agar backup plate | Nov. 12, 2007 |
| Transfer to Research Archives | Mar. 13, 2008 |
| Transfer to Liquid Archival Tubes | Sep. 15, 2008 |
| Start of continuous liquid culture | May 20, 2008 |
| Production of Q agar slants | Oct. 20, 2008 |
| Transfer to GLP Bioproduction | TBD |

Phytoplasma and Bioburden Testing.

BAP12-B2-150 was negative for the presence of phytoplasma using a nested PCR protocol and also negative for the presence of bioburden.

Preparation of BAP12-B2-150 9° C. Research Q-Slants.

BAP12-B2-150 obtained from the Research Archive was prepared in 50 ml of Media 1.2 and has been in continuous liquid culture since that time. IV cultures are grown at 25° C. in continuous light of ˜250 μmol/m²/second (using a 2D light meter) and are transferred every ten to fourteen days. The continuous liquid stock has been used as a source of inoculum for all Upstream Process Development work and continues to be stable for the production of PLG protein.

To grow inoculum for the BAP12-B2-150 Q-slants, three healthy and green multiple frond clusters from the continuous liquid stock were used to inoculate IVs containing 50 ml of Media 708 (Media 1.2 supplemented with 100 μM propyl gallate and 10 mM L-glutamine). To propagate Q-slant batches the same protocol listed here is followed using three-frond clusters from a Q-slant of the previous Q-slant batch as the inoculum source rather than the continuous liquid culture stock. IVs were covered with Milliwrap and incubated, in a Percival or growth chamber, for 10 days at 250 μmol/m²/second of continuous light and a temperature of 25° C.

IVs were then placed in a Percival or growth chamber in low light ˜50 μmol/m²/second and a temperature of 23° C. for a second incubation of 4 days. Following the low light incubation the IVs are used to make BAP12-B2-150 Q-slants.

Agar slants containing Media 707 (Hoagland's medium supplemented with 100 μM propyl gallate and 10 mM L-glutamine) were inoculated with 18 to 20 three-frond clusters from the prepared inoculum cultures. Slants were fitted with a Q-slant top and stored at 9° C. in a low light level of 5 to 19 μmol/m²/second, and their health and stability were monitored over time.

Growth of line BAP12-B2-150 and PLG expression from IVs inoculated from a 9° C. Q agar slant. IVs were inoculated on different days, and replicate IVs were also inoculated on the same day. IVs were also harvested after sixteen and eighteen days of growth. The growth and PLG protein levels per grams fresh weight of BAP12-B2-150 in an IV with 50 ml of Media 1.2, grown in a Percival with continuous light of 250 μmol/m2/second (measured by 2D light meter) 3 three-frond clusters as inoculum (<0.05 gram) is shown below in Table 6.

TABLE 6
Increase in biomass and the plasminogen specific activity (plasminogen in μgs per gram
fresh weight) following growth of BAP12-B2-150 IVs for sixteen or eighteen days.

| | | | | | Fresh | Total | Total |
|---|---|---|---|---|---|---|---|
| | Source of | | | | weight in | plasminogen | plasminogen |
| | inoculum | | | | grams of | activity in μgs | activity in μgs |
| | Q agar | | Start | Growth | tissue at | per 250 μgs of | per gram |
| Line | slant # | Replicate | (day) | (days) | harvest | tissue | fresh weight |
| BAP12- | 49 | 1 | 1 | 16 | 0.958 | 134.53 | 538.1 |
| B2-150 | | | | | | | |
| BAP12- | 49 | 2 | 1 | 16 | 1.023 | 130.61 | 522.42 |
| B2-150 | | | | | | | |
| BAP12- | 49 | 3 | 1 | 16 | 0.982 | 152.02 | 608.068 |
| B2-150 | | | | | | | |
| BAP12- | 49 | | 1 | 18 | 1.141 | 172.04 | 688.176 |
| B2-150 | | | | | | | |
| BAP12- | 49 | | 2 | 16 | 0.926 | 140.70 | 562.812 |
| B2-150 | | | | | | | |
| BAP12- | 46 | 1 | 1 | 16 | 1.078 | 158.22 | 632.888 |
| B2-150 | | | | | | | |
| BAP12- | 46 | 2 | 1 | 16 | 1.207 | 124.75 | 498.996 |
| B2-150 | | | | | | | |
| BAP12- | 46 | 3 | 1 | 16 | 0.901 | 151.69 | 606.748 |
| B2-150 | | | | | | | |
| BAP12- | 46 | | 1 | 18 | 1.394 | 137.87 | 551.48 |
| B2-150 | | | | | | | |
| BAP12- | 46 | | 2 | 16 | 0.966 | 130.73 | 522.912 |
| B2-150 | | | | | | | |
| CNTD02- | Research | 1 | 1 | 16 | 0.699 | 0.57 | 2.28 |
| B1-200 | Archive | | | | | | |
| CNTD02- | | 2 | 1 | 16 | 1.077 | 0.62 | 2.46 |
| B1-200 | | | | | | | |
| CNTD02- | | 3 | 1 | 16 | 0.991 | 1.18 | 4.732 |
| B1-200 | | | | | | | |
| BAP12- | 52 | 1 | 2 | 16 | 0.695 | 140.54 | 562.152 |
| B2-150 | | | | | | | |
| BAP12- | 52 | 2 | 2 | 16 | 0.824 | 99.98 | 399.928 |
| B2-150 | | | | | | | |
| BAP12- | 52 | 3 | 2 | 16 | 1.085 | 123.81 | 495.24 |
| B2-150 | | | | | | | |
| BAP12- | 52 | | 3 | 18 | 1.419 | 163.68 | 654.724 |
| B2-150 | | | | | | | |
| BAP12- | 47 | 1 | 3 | 16 | 1.005 | 138.34 | 553.376 |
| B2150 | | | | | | 135.47 | 541.884 |
| | | | | | | 130.80 | 523.184 |
| BAP12- | 47 | 2 | 3 | 16 | 1.34 | 127.54 | 510.144 |
| B2-150 | | | | | | 135.36 | 541.428 |
| | | | | | | 152.25 | 609.016 |
| BAP12- | 47 | 3 | 3 | 16 | 1.152 | 145.86 | 583.42 |
| B2-150 | | | | | | 149.76 | 599.032 |
| BAP12- | 47 | | 1 | 16 | 1.024 | 116.78 | 467.116 |
| B2-150 | | | | | | 149.63 | 598.516 |
| | | | | | | 153.20 | 612.816 |

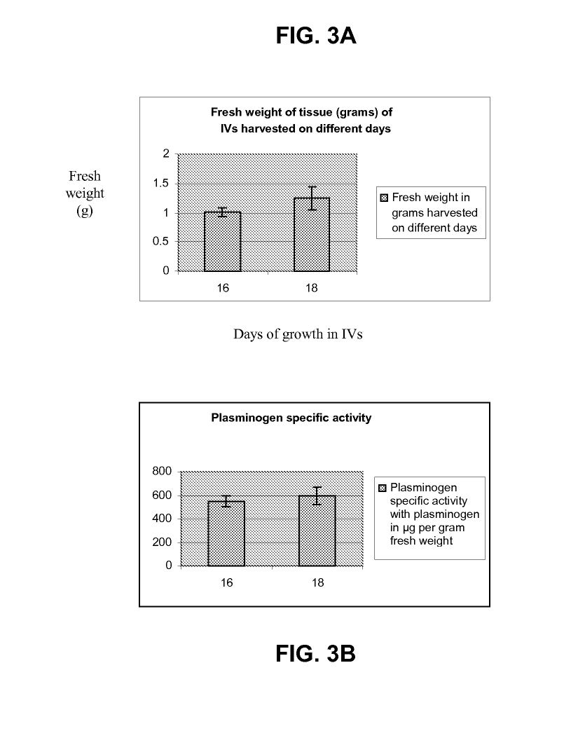

As shown in Table 6 and FIG. 3A, after 16 days growth the lowest biomass was 0.695 grams and the highest biomass was 1.34 grams. At 18 days, the lowest biomass growth was 1.024 grams and the highest biomass was 1.394 grams.

As shown in FIG. 3B, total PLG accumulation in the tissue for line BAP12-B2-150 was measured by plasmin activity assay to be between 400 and 680 μg per gram of fresh weight tissue for different tissue samples harvested on different days.

Plasminogen Characterization

All characterization of PLG was done on either crude extracts or partially purified material. Partially purified PLG was generated by Lysine-Sepharose chromatography. PLG from Lysine-Sepharose eluate co-purified with PLG fragments that contained all or parts of the kringle 1-5 domain. PLG was characterized by Western Blot, SDS-PAGE, and activity analysis as determined by the chromogenic assay described above.

Western Blot of Non-Reduced SDS PAGE

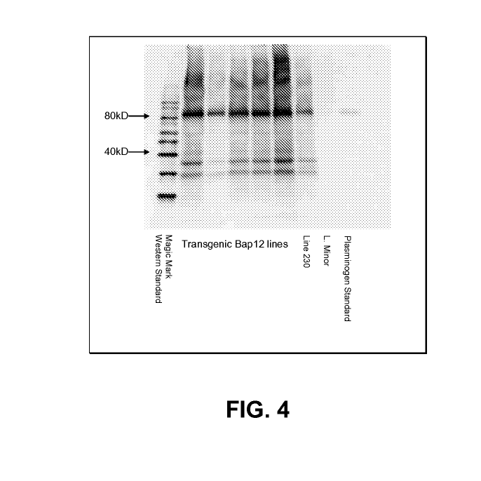

As shown in FIG. 4, full-length PLG co-migrates with the human reference PLG as a 90 kDa band by Western blot. Additional fragments containing immunoreactive PLG fragments are also present in the material.

SDS-PAGE of Non-Reduced Lysine Sepharose Purified PLG

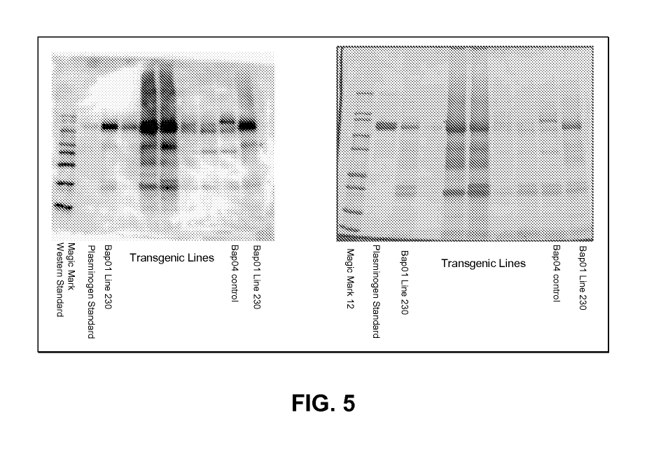

As shown in FIG. 5, all protein bands identified in the SDS PAGE also were detected in the Western blot indicating that all detectable protein was PLG related.

Plasmin Characterization

Plasmin was purified as described above and was characterized by the following assays: SDS-PAGE purity; specific activity; SEC; inhibition by α₂-AP (i.e., inhibition via complex formation and inhibition via bioactivity); fibrinolysis; peptide map analysis; plasmin analysis using RP chromatography; and stability analysis.

SDS-PAGE Purity:

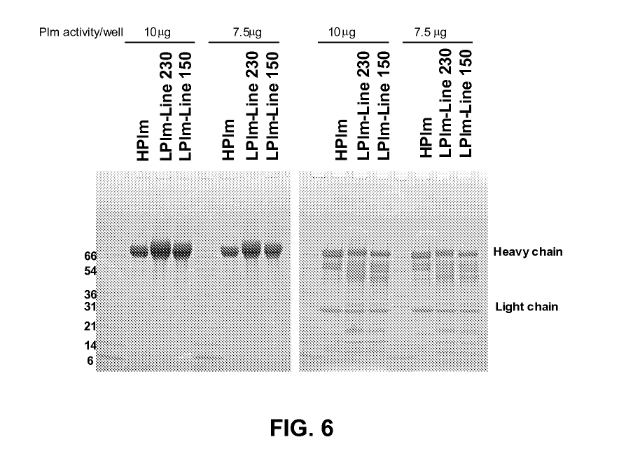

As shown in FIG. 6, non-reducing SDS-PAGE of human Lemna-derived plasmin from BAP12-B2-150 (transgenic line expressing mature human plasminogen with the N289D substitution; see SEQ ID NO:6) was analyzed by SDS-PAGE along side plasmin from line 230 (transgenic line expressing mature human plasminogen of SEQ ID NO:4, which has the N-linked glycosylation site Asn-289; GLP production) and human reference plasmin. Side by side comparison of the samples shows comparable molecular weights. The gels were 4-20% gradients and were stained with Coomassie blue.

Specific Activity:

Plasmin concentrations were determined by activity analysis and were also derived from absorbance at 280 using an extinction coefficient of 2. The ratio of active plasmin versus total protein (specific activity) was 65% for plasmin derived from BAP01-B2-230 and 68% for plasmin derived from BAP12-B2-150.

Size Exclusion Chromatography (SEC):

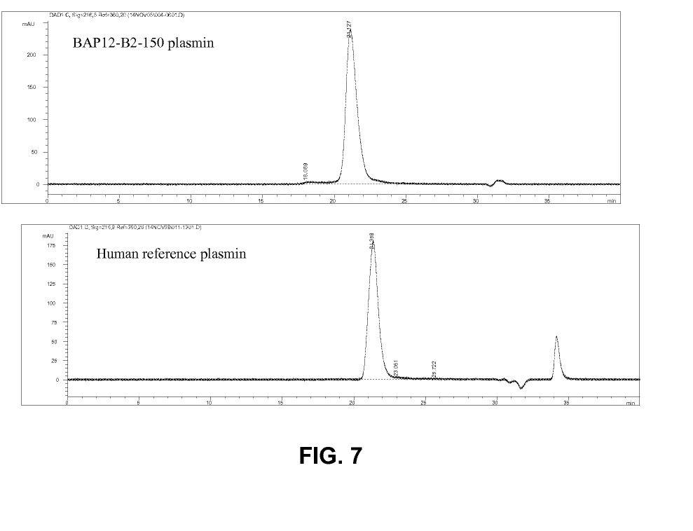

SEC was performed on plasmin derived from BAP12-B2-150 and human reference plasmin according to SOP AD-078Q. As shown in FIG. 7, SEC revealed no degradation products and no detectable aggregates in the BAP12-B2-150 purified material.

Inactivation by α₂-Antiplasmin (SDS-PAGE):

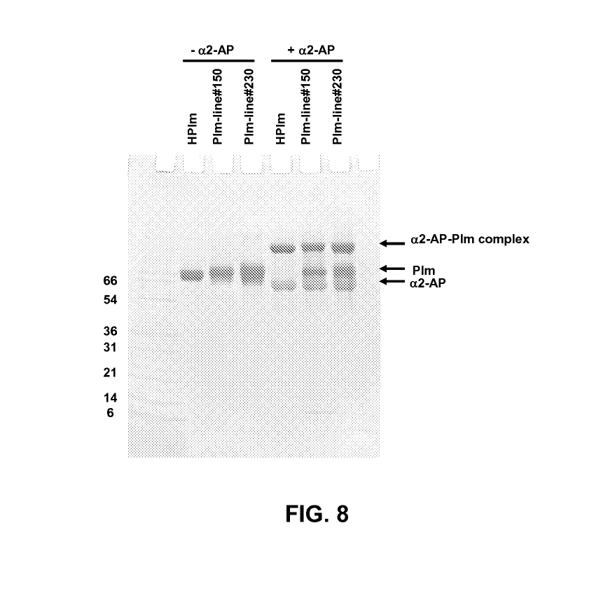

As shown in FIG. 8, human or Lemna derived plasmin alone, under non-reducing conditions, ran as a single band. Human plasmin when mixed with α2-AP displayed two bands. A species with a higher apparent molecular weight than plasmin corresponding to plasmin:α₂-AP complex, and a species corresponding to free α₂-AP. A plasmin:α₂-AP complex with the same MW was also formed with plasmin derived from both transgenic line BAP12-B2-150 and transgenic line BAP01-B2-230. In addition to the complex, a part of Lemna-derived plasmin remained and did not form complex. This particular plasmin is inactive and is thought to be the reason for the low specific activities of Lemna-derived plasmin reported earlier (65%-68%). The excess of α₂-AP visible on the gel is consistent with this conclusion.

Inactivation of Plasmin Activity by α₂-AP (Chromogenic Activity Assay):

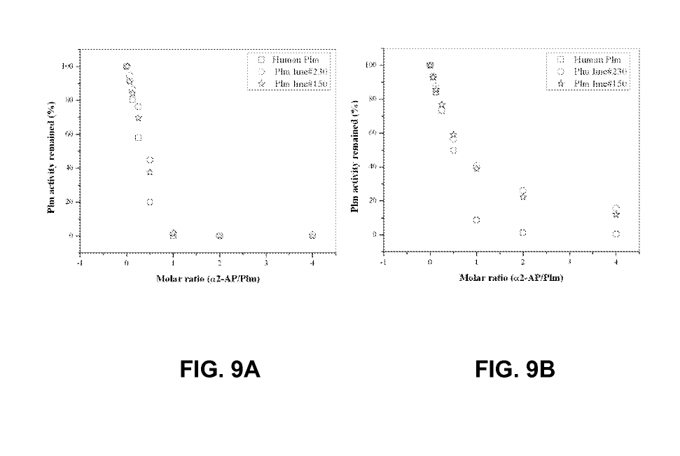

Two different experiments were performed to address inhibition of plasmin activity by α₂-AP. In the first design, the goal was to test if 100% inhibition was achieved at the expected 1:1 (plasmin:α₂-AP) molar ratio. In the second design, the goal was to test if plasmin derived from the two different plant lines was inhibited at the same rate. To answer the first question, constant plasmin concentration was incubated with different concentrations of α₂-AP. After 30 minutes at room temperature, the remaining plasmin activity was quantified by adding the substrate S-2403 and by monitoring the reaction at 405 nm. The data are presented in FIG. 9A.

To answer the question related to rates of inhibition, α₂-AP at different concentrations was mixed first with the same amount of the substrate S-2403. Then, the same amount of plasmin was added right before starting the monitoring of the reaction. The data are presented in FIG. 9B. At plasmin:α₂-AP molar ratio of 1:1, no plasmin activity was detected for human, BAP12-B2-150, or BAP01-B1-230 plasmin, indicating that the inhibition was complete (see FIG. 9A).

For the kinetics of plasmin inhibition, the assay was designed to appreciate visually the rates of inhibition. There are two conclusions to draw from FIG. 9B. First, within the time frame of setting up the assay, at the same concentrations, human plasmin required less α₂-AP to reach 100% inhibition. In other words, in the setting where plasmin was mixed quickly to human plasmin, the same amount of α₂-AP would inhibit more of the human than the Lemna-derived plasmin. When plasmin was not pre-incubated with α₂-AP, at a molar ratio of 1:1, about 90% of human plasmin was inhibited; while only 60% of Lemna derived-plasmin was inhibited. Second, plasmin derived from the two plant lines was indistinguishable. Therefore, glycosylation of plasmin at the Asn at residue 212 (Asn-212 of plasmin corresponds to Asn-289 of the mature human plasminogen sequence) does not seem to be an important factor in α₂-AP mediated inhibition.

Fibrinolysis:

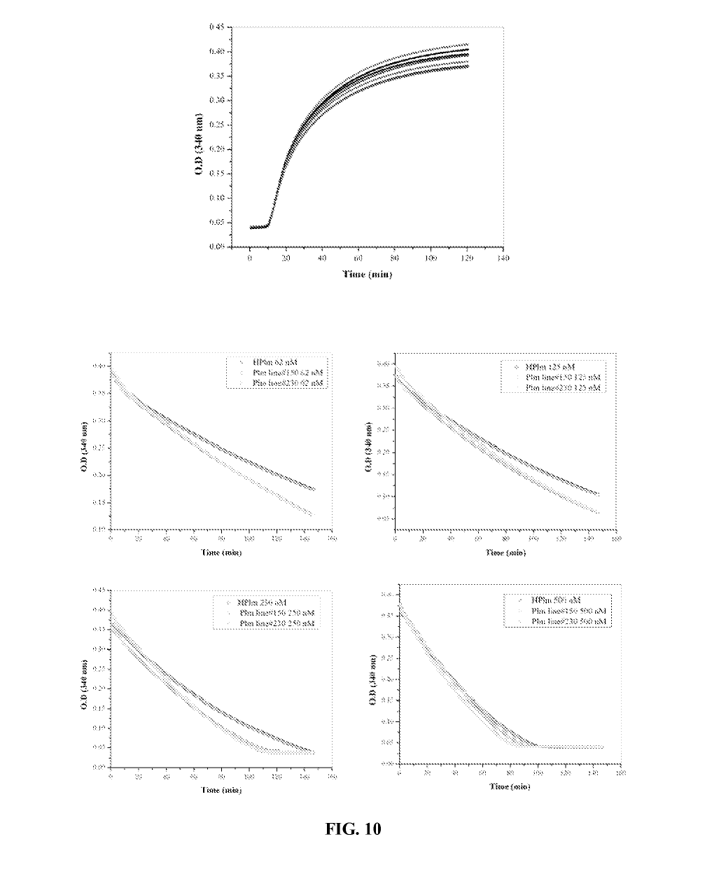

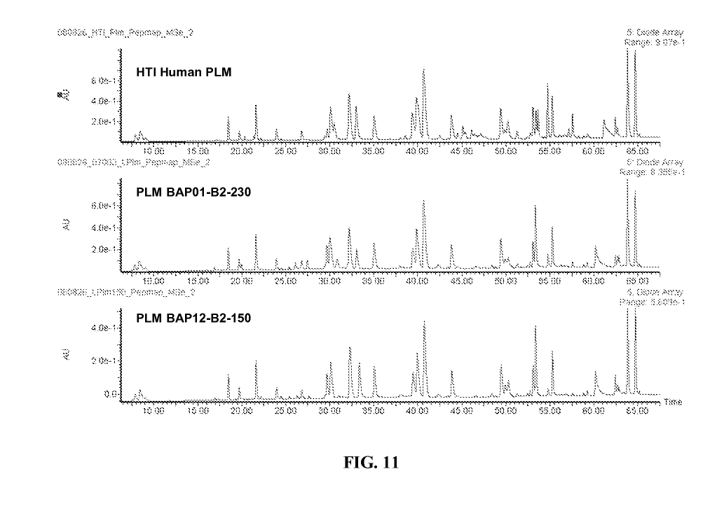

Fibrinolytic activity of human reference plasmin and Lemna-derived plasmin from transgenic lines BAP01-B2-230 and BAP12-B2-150 was measured as described above. The fibrin formation profiles and the O.D 340 nm as a function of time were very reproducible and reached comparable final absorbance values. The range of plasmin concentrations that was chosen was adequate as evidenced by the steeper slopes of the curves with increasing plasmin concentrations, as shown in FIG. 10. There was no evidence of a difference between fibrinolysis induced by plasmin derived from BAP01-B2-230 and BAP12-B2-150; their respective lysis profiles were very similar for all four plasmin concentrations.

Peptide Mapping: Representative RP-HPLC profile of LysC-digested human reference plasmin and Lemna-derived plasmin from transgenic lines BAP01-B2-230 and BAP12-B2-150, showing A216 and MS ionization traces (17) are shown in FIG. 11. Each observed peak was identified by MS as a derivative of the parent plasmin molecule. The identified peaks represented a primary sequence amino acid coverage exceeding 95%. A plasma-derived reference standard was used comparatively to confirm the presence of the main RP-HPLC peaks. The peptide map analysis shows that the glycosylated site Asn identified in peptide H16 in line BAP01-B2-230 was successfully mutated to Asp in the plant line BAP12-B2-150. In line BAP01-B2-230, this residue is partially glycosylated but not at all in line BAP12-B2-150. No other differences were observed by comparing the peptide maps.

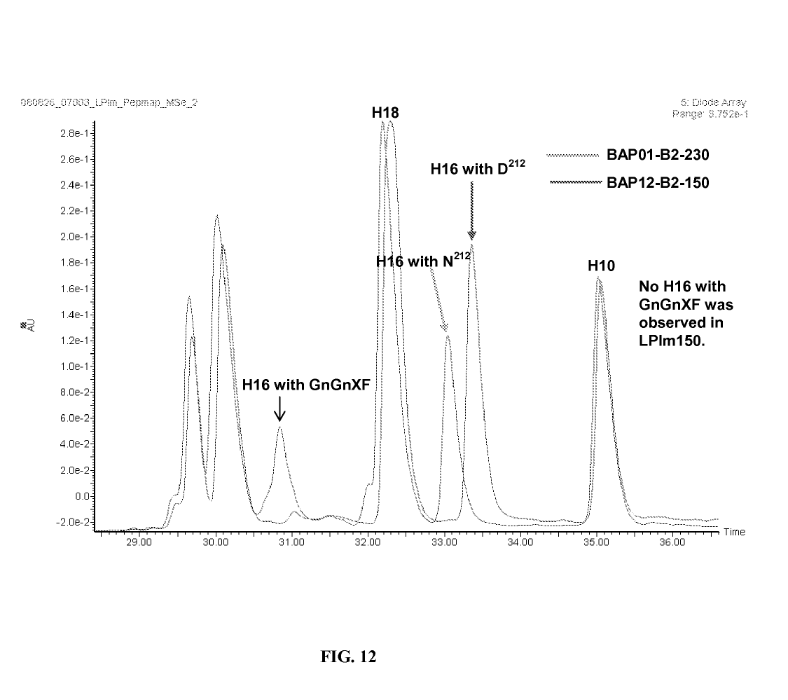

FIG. 12 is a zoom of the chromatogram shown in FIG. 11 that highlights the peaks uncommon to the plasmin derived from the two plant lines, BAP12-B2-150 and BAP01-B2-230. The three peaks were identified as peptide, H16, in which the Asn residue in BAP01-B2-230 (Asn-212 or N²¹²) was changed to Asp (Asp-212 or D²¹²) in BAP12-B2-150. In line BAP01-B2-230, the Asn-212 residue was partly glycosylated.

Plasmin Analysis Using Reverse-Phase (RP) Chromatography:

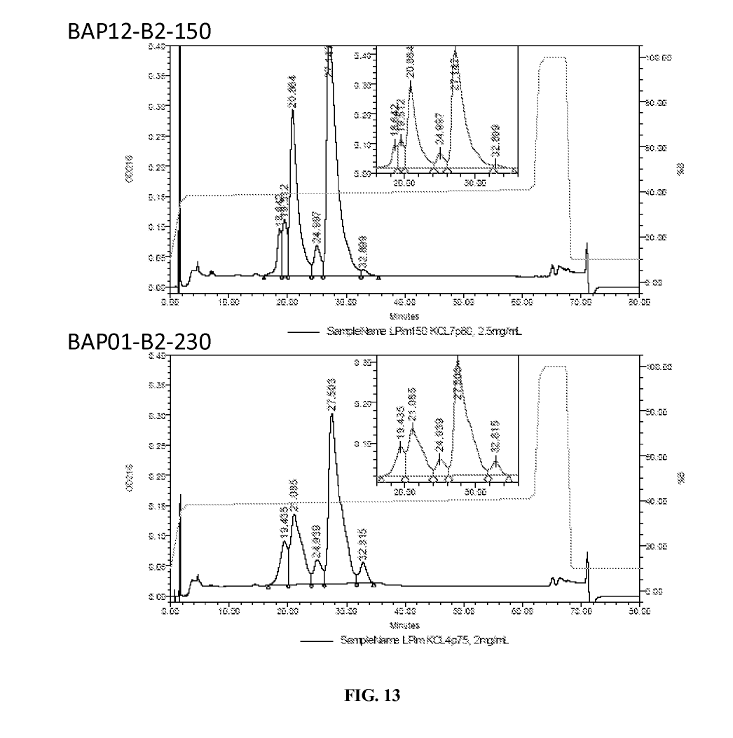

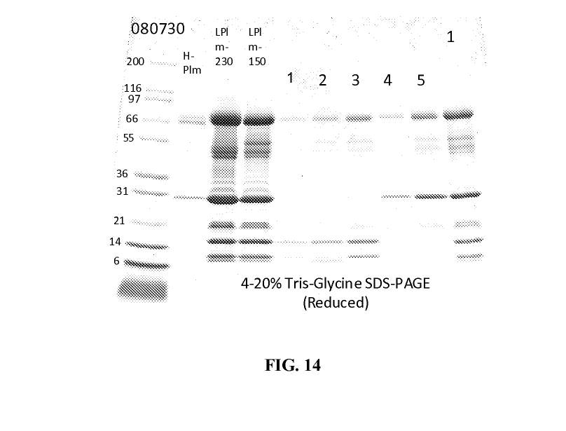

Reverse-phase assay utilized chromatography on a C18 RP column to separate the many forms of plasmin in the final formulated preparation. As shown in FIG. 13, plasmin purified from both transgenic line BAP01-B2-230 and transgenic line BAP12-B2-150 separated into five peaks. The peaks were collected and analyzed by SDS-PAGE (FIG. 14) and plasmin activity assay. The majority of the activity was observed in peaks #4 and #5 from the RP chromatogram (FIG. 13). The majority of the full-length plasmin was observed in peaks #4 and #5 as the 31 kD and 66 kD proteins from the RP chromatogram (FIG. 14). These data show similar results for both the transgenic line BAP01-B2-230 and transgenic line BAP12-B2-150 material.

Freeze-Thaw Stability:

The activity data after one freeze-thaw cycle and 3 freeze-thaw cycles is summarized below in Table 7.

TABLE 7
Freeze-thaw stability results.

| | Initial | Concentration after | |
|---|---|---|---|
| Sample | Concentration | 3 freeze-thaw cycles | % change |
| HTI PLM | 2.34 mg/ml | 2.36 mg/ml | 1.0 |
| BAP12-B2-150 | 2.13 mg/ml | 2.06 mg/ml | 3.3 |

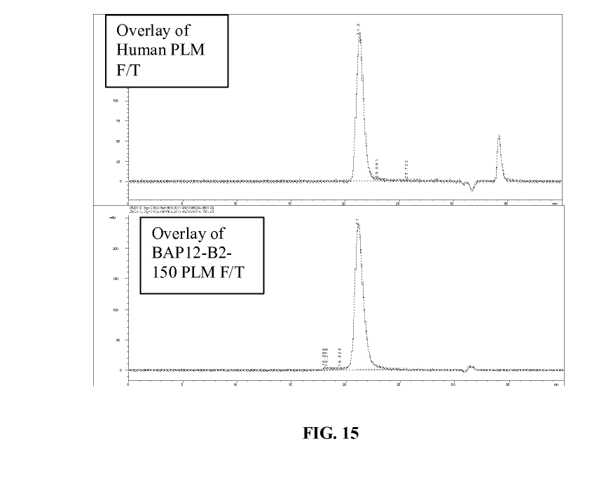

The concentrations of plasmin activity before and after the three cycles of freeze thaw are within 5%, which are not significant differences. As shown in FIG. 15, overlays of the SEC chromatograms showed no new degradation products and no aggregates were formed after the freeze-thaw treatment of the plasmin. This conclusion applies for both BAP12-B2-150 and human reference plasmin.

Stability of BAP12-B2-150 at Ambient Temperature and at 4° C.:

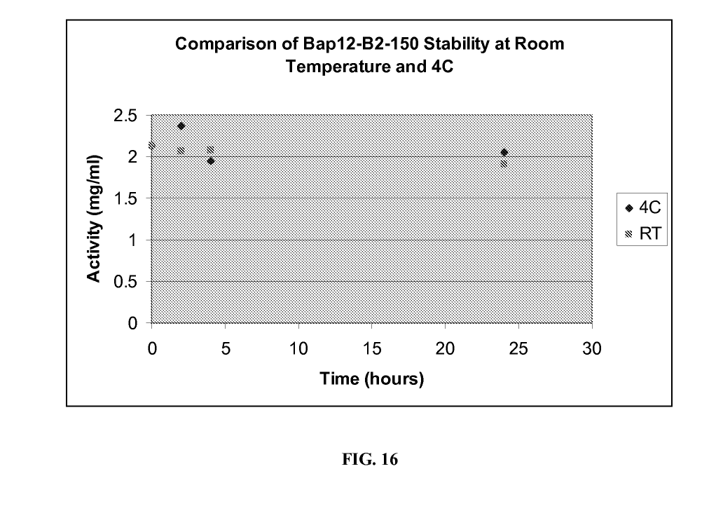

The activity data for the plasmin stability at ambient temperature and 4° C. is summarized below in Table 8 and as a graphical representation in FIG. 16.

TABLE 8
Summary of plasmin activity of plasmin derived from line BAP12-B2-
150 upon storage at ambient temperature and at 4° C.

| | Activity (mg/ml) | Activity (mg/ml) |
|---|---|---|
| Time Point (hours) | 4° C. | RT |
| 0 | 2.13 | 2.13 |
| 2 | 2.37 | 2.06 |
| 4 | 1.95 | 2.08 |
| 24 | 2.05 | 1.91 |
| 168 | 2.01 | N/A |

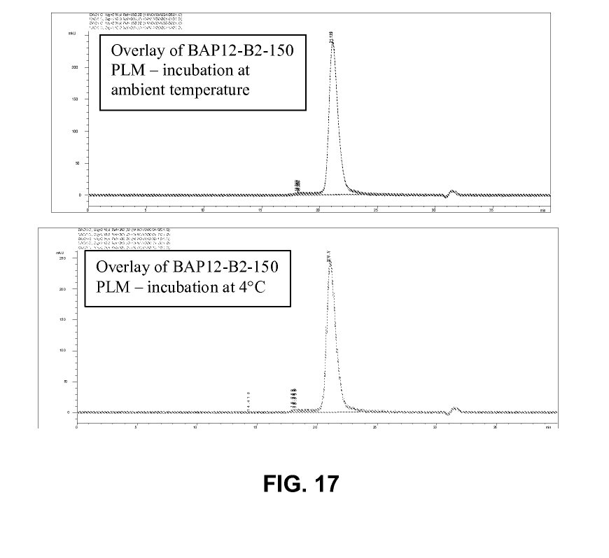

FIG. 17 shows overlays of SEC chromatograms obtained after incubation at ambient temperature and at 4° C. for plasmin derived from transgenic line BAP12-B2-150. BAP12-B2-150 plasmin activity was stable at 4° C. and ambient temperature over a period of 24 hours. The activity of the sample kept at 4° C. for 1 week was similar to the time zero analysis. The SEC data showed no degradation products and no detectable aggregates in BAP12-B2-150 plasmin stored a 4° C. for up to one week and at 24° C. for 24 hours.

Plasmin derived from transgenic line BAP12-B2-150 was therefore fully characterized in comparison to human reference plasmin, and the results have been compared to previous results obtained from transgenic line BAP01-B2-230. The main difference between the plasmin derived from BAP12-B2-150 and plasmin derived from BAP01-B2-230 or human reference plasmin is that the BAP12-B2-150 plasmin was engineered to be produced without glycosylation. This was confirmed by peptide map analysis.

All other properties of the BAP12-B2-150 plasmin are similar to those observed with BAP01-B2-230 and human reference plasmin. This includes purity, specific activity, inhibition of plasmin activity with α₂-AP, fibrinolysis, and RP-chromatography. In addition, the BAP12-B2-150 line showed similar limited stability characteristics as observed with the BAP01-B2-230 line.

### Example 2

### Comparison of Plasminogen Yield from BAP01-B2-230 and BAP12-B2-150 Transgenic Lines

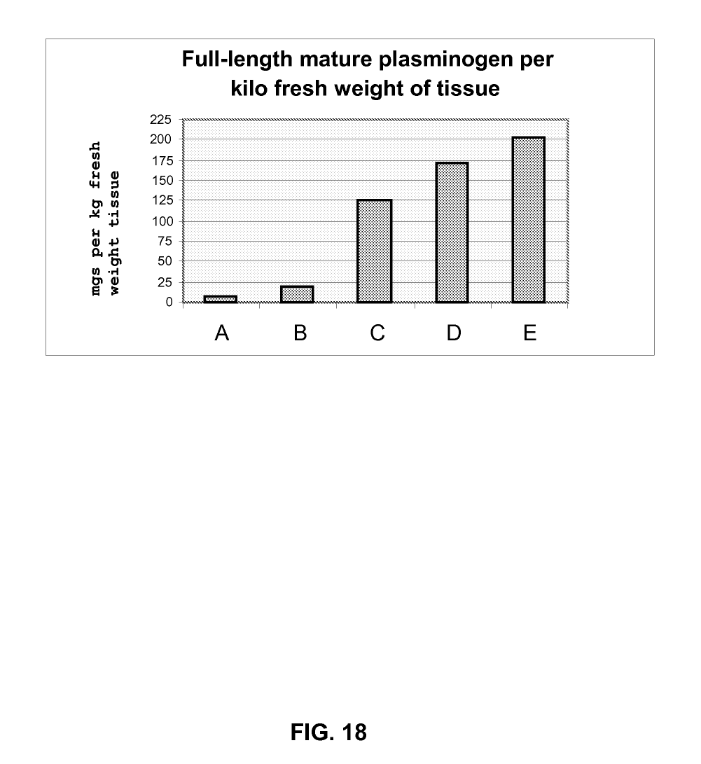

Yield of full-length (not truncated) mature plasminogen from the transgenic Lemna line expressing mature human PLG with the Asn-289 N-glycosylation site (BAP01-B2-230) and the transgenic line expressing mature human PLG with the N289D substitution was compared under varying culture conditions and culture times. The results are shown in FIGS. 18 and 19. Transgenic line BAP01-B2-230 was cultured for 21 days (bar A in FIGS. 18 and 19) or 28 days (bar B in FIGS. 18 and 19) at 24.5° C. in passive vented SV. Transgenic line BAP12-B2-150 was cultured for 28 days (bar C in FIGS. 18 and 19) at 24.5° C. in passive vented SV, or for 21 days (bar D in FIGS. 18 and 19) or 33 days (bar E in FIGS. 18 and 19) in a vented FASV at 21° C. Yield of full-length mature PLG in mg per kilogram fresh weight of tissue or yield in mg per growth vessel are shown under the various culture conditions in FIG. 18 and FIG. 19, respectively. It can be seen from these figures that significant increases in yield of full-length mature PLG were obtained from transgenic line BAP12-B2-150.

Thus, the yield of full-length mature plasminogen in mg per kg fresh weight of tissue was 125.4, 172, and 203.3 for transgenic line BAP12-B2-150 when cultured for 28 days in a passive vented SV at 24.5° C. (bar C, FIG. 18), for 21 days in a vented FASV at 21° C. (bar D, FIG. 18), and for 33 days in a vented FASV at 21° C. (bar E, FIG. 18), respectively. In contrast, the yield of full-length mature plasminogen in mg per kg fresh weight of tissue was only 8.2 and 20.2 for transgenic line BAP01-B2-230 when cultured for 21 days in passive vented SV at 24.5° C. (bar A, FIG. 18) and for 28 days in passive vented SV at 24.5° C. (bar B, FIG. 18), respectively. Under comparable growth conditions (see bar B versus bar C, FIG. 18), yield of full-length mature plasminogen in mg per kg fresh weight of tissue was increased by about 6-fold in transgenic line BAP12-B2-150.

The yield of full-length mature plasminogen in mg per growth vessel was 10.78, 12.2, and 21.2 for transgenic line BAP12-B2-150 when cultured for 28 days in a passive vented SV at 24.5° C. (bar C, FIG. 19), for 21 days in a vented FASV at 21° C. (bar D, FIG. 19), and for 33 days in a vented FASV at 21° C. (bar E, FIG. 19), respectively. In contrast, the yield of full-length mature plasminogen in mg per growth vessel was only 1.1 and 2.8 for transgenic line BAP01-B2-230 when cultured for 21 days in passive vented SV at 24.5° C. (bar A, FIG. 19) and for 28 days in passive vented SV at 24.5° C. (bar B, FIG. 19), respectively. Under comparable growth conditions (see bar B versus bar C, FIG. 19), yield of full-length mature plasminogen in mg per growth vessel was increased by about 3.8-fold in transgenic line BAP12-B2-150.

These data show that significant increases in yield of full-length mature PLG can be obtained from Lemna when the PLG is expressed in an aglycosylated form.

### Example 3

### Demonstration of Fibrin Binding of BAP12-B2-150 Plasmin

Fibrin binding of the aglycosylated plasminogen is indicated by binding to Lysine Sepharose. Lysine Sepharose is routinely used for kringle domain containing proteins. Confirmation of fibrin binding is determined in one of two ways. A direct binding affinity measurement is obtained via the use of Biacore with a fibrin-coated chip. A similar experiment is run in a microtiter plate containing fibrin. In this study, aglycosylated plasminogen from transgenic line BAP12-B2-150 is applied to the plate and unbound protein is washed away. Fibrinolysis is then measured by activation of the aglycosylated plasminogen to aglycosylated plasmin. Only plasminogen bound to the fibrin is available to lyse the fibrin clot. A comparison is made to glycosylated PLG from transgenic line Transgenic line BAP01-B2-230.

The BAP12-B2-150 plasmin shows fibrin binding similar to that of the glycosylated BAP01-B2-230 plasmin, even in the absence the native N-linked glycan at the amino acid residue corresponding to Asn-289 of mature human PLG, and the absence of the native O-linked glycans at the amino acid residues corresponding to residues Ser-249 and Thr-346 of the mature PLG polypeptide. The native N-linked and O-linked glycosylation sites occur within or between the kringle domains that are responsible for fibrin binding. It is surprising that the fibrin-binding properties of the aglycosylated BAP12-B2-150 plasmin are not affected by the lack of these N-linked and O-linked glycans.

### Example 4

### Stability Studies of Plasmin in Different Formulation Conditions

A series of studies were carried out to examine the stability of plasmin. Although the following studies were with glycosylated human plasmin (H-Plm) and glycosylated Lemna-derived plasmin (L-Plm), similar stability would be found with Lemna-derived aglycosylated plasmin. Stability was measured by plasmin activity as determined by the hydrolysis of the colorimetric substrate S-24032. This substrate has been designed to possess selectivity similar to natural substrates of plasmin.

### Stability of H-Plm in Buffers of pH 2 Through 11.

Low ionic strength buffers were used and incubation was carried at room temperature for 6 to 8 hrs. At room temperature, H-Plm appears to be most stable between pH 2.0 and 4.0, losing about 20% of its activity over 6 hr. There is a dramatic decrease in stability between pH 4 and 5. H-Plm was most stable at pH 4.0, losing less than 10% of its activity within 8 hr. It was also very stable at pH 3.8 and 4.2 where it lost about 10% of its activity over 6 hr. H-Plm lost activity at a constant rate at all of the pHs tested between 7 and 11.

pH-Dependent Activity of Human Plasmin (H-Plm).

Commercial H-Plm in buffers from pH 4.0 to 12.0 was assayed in order to determine the pH at which activity is highest. Under room temperature conditions, the pH activity profile for H-Plm resembled a typical bell-shaped curve with the apex or pH optimum at around 8.0. H-Plm retained greater than 80% of its activity between pH 7.0 and 9.0. There was no detectable activity at either pH 4.0 or 12.0.

### Stability of Lemna Plasmin at pH 3.7.

Lemna plasminogen (L-Plg) was activated and the stability of Lemna-derived plasmin (L-Plm) was evaluated after dialysis in acetic acid acidified water at pH 3.7 held on ice. In this experiment 5.2 mg (54.74 nM) of L-Plg (purified from crude tissue extracts by Lysine-Sepharose) was activated using 100 μg (1.85 nM) of human urokinase in 25 mM Tris/25 mM lysine/100 mM NaC1/50% glycerol at room temperature for 7.5 hr. The reaction was diluted to 25% glycerol with buffer and applied to a benzamidine column. Fractions were analyzed by SDS-PAGE and silver stain. Two mL of fraction 11 (about 1.2 mg) was dialyzed against 5 L of 9° C. water acidified to pH 3.7 with acetic acid for 24 hr. The volume after dialysis had increased more than 3-fold to 6.5 mL. L-Plm lost about 30% of its activity within two days, but remained stable at about 70% of its original activity for up to 16 days at which time the study ended. Without being bound by any theory or mechanism of action, the 30% loss could be an artifact of the T=0 sample that was frozen for 120 hr prior to thawing and activity measurement.

Stability of L-Plm in Water Acidified with Acetic Acid to pH 3.7.

The effect of 25% glycerol on plasmin stability at room temperature and at different pH's was determined over a 20-hour period. Using human plasmin, plasmin solutions were prepared from pH 4 to 11 and supplemented with 25% glycerol. Plasmin appeared to be most stable around pH 8.0 and least stable at pH 5.0. At 48 hours, degradation appeared to be pH selective, in that the kringle domains degraded more readily as pH increased from 8 to 11, while the catalytic domain exhibited the greatest fragmentation in acidic pH, especially at pH 5.

### Stability of Lemna Plasmin at pH 4, 5, 8 and 9 in Either 9% or 25% Glycerol.

The effect of 9% or 25% glycerol on Lemna-derived plasmin stability at different pH's was determined. Enzymatic activity was evaluated over time. Plasmin appeared to be most stable in more glycerol (25%) at pH 8 or 9 and less so at pH 5 without regard to the amount of glycerol. As in the previous study above, degradation appeared to be pH selective and the level of activity corresponded to the intensity of the catalytic domain band.

### Stability of Plasmin in 10, 20, 30, 40 and 50% Glycerol at pH 7.5.

The stability of H-Plm in 10, 20, 30, 40 and 50% glycerol at pH 7.5 was evaluated. pH 5.0 was used also to evaluate the protective effects of glycerol because plasmin degrades rapidly and produces multiple fragmentation bands at this pH.

The following table lists the recipes for the solutions used in this study containing glycerol, respectively. Glycerol was added to 15 mL plastic tubes using a positive displacement 1 mL pipette.

pH 5.0 and 7.5 buffers with glycerol:

For pH 5.0, titrate with 2.0N NaOH (about 160 uL)

| Per- | Glyc- | | Acetic | | | Final |
|---|---|---|---|---|---|---|
| cent- | erol | Glycerol | Acid | 5M NaCl | | Glycerol |
| age* | (gm) | (mL) | (uL) | (uL) | Water | % |
| 1.3 | 0.16 | 0.13 | 28.6 | 300 | to 10 mL | 10 |
| 13.5 | 1.70 | 1.35 | 28.6 | 300 | to 10 mL | 20 |
| 25.7 | 3.23 | 2.57 | 28.6 | 300 | to 10 mL | 30 |
| 37.8 | 4.75 | 3.78 | 28.6 | 300 | to 10 mL | 40 |
| 50.0 | 6.29 | 5.00 | 28.6 | 300 | to 10 mL | 50 |

| | | |
|---|---|---|
| Final | 50 mM | 150 mM |
| Concen- | | |
| tration | | |

For pH 7.5, titrate with 2.0N NaOH about 10 uL)

| | | | 500 mM | | | |
|---|---|---|---|---|---|---|
| Per- | Glyc- | | Phos- | | | Final |
| cent- | erol | Glycerol | phate | 5M NaCl | | Glycerol |
| age* | (gm) | (mL) | (uL) | (uL) | Water | % |
| 1.3 | 0.16 | 0.13 | 500 | 300 | to 10 mL | 10 |
| 13.5 | 1.70 | 1.35 | 500 | 300 | to 10 mL | 20 |
| 25.7 | 3.23 | 2.57 | 500 | 300 | to 10 mL | 30 |
| 37.8 | 4.75 | 3.78 | 500 | 300 | to 10 mL | 40 |
| 50.0 | 6.29 | 5.00 | 500 | 300 | to 10 mL | 50 |

| | | |
|---|---|---|
| Final | 50 mM | 150 mM |
| Concen- | | |
| tration | | |

*Percentage is based on adding 21.4 μL of 5.6 mg H-Plm/mL 50% glycerol to 98.6 μL of test buffer to achieve final glycerol concentrations of 20%, 30%, etc.

For stability testing, the pH test buffers were chilled initially on ice. T=0 was designated when, after the plasmin was added to the pH test buffer while on ice, it was removed from the ice and placed at room temperature. At this time, a 10 μL aliquot was taken and added immediately to 990 μL TBST (50 mM Tris/150 mM NaC1/0.05% Tween 20/pH 8.0) in 1.5 mL tubes, vortexed for 5 sec, and then immediately assayed for plasmin activity.

The plasmin assay was as follows (components added in sequence):
a. 50 μL of TBST had been added to each well first.
b. 50 μL of 100× diluted sample (e.g., 10 μL sample+990 μL TBST) was then added to the 50 uL TBST in the well.
c. 50 μL of substrate (8.4 mg S-2403+5.5 mL water) was added, bubbles were dispersed with a flame, and the plate was read at 405 nm for 2 min at 10 s intervals in order to obtain the initial rate in milliunits per minute. The mixtures were assayed at t=0, 1, 2, 4, 8, and 24 hours at room temperature (RT). Each test sample was assayed in duplicate.

For SDS-PAGE samples, 5 μL, of the 1 mg H-Plm sample was added to 10 μL of 2×SDS-PAGE buffer w/100 mM DTT. Samples were frozen at −80° C. until the gel was run. Samples were heated at 95° C., and 15 μL (5 μg of protein) were loaded per lane.

At concentrations greater than 20%, there is very little activity loss with glycerol at pH 7.5 over 8 hrs.

Many modifications and other embodiments of the inventions set forth herein will come to mind to one skilled in the art to which these inventions pertain having the benefit of the teachings presented in the foregoing descriptions and the associated drawings. Therefore, it is to be understood that the inventions are not to be limited to the specific embodiments disclosed and that modifications and other embodiments are intended to be included within the scope of the appended claims and list of embodiments disclosed herein. Although specific terms are employed herein, they are used in a generic and descriptive sense only and not for purposes of limitation.

All publications and patent applications mentioned in the specification are indicative of the level of those skilled in the art to which this invention pertains. All publications and patent applications are herein incorporated by reference to the same extent as if each individual publication or patent application was specifically and individually indicated to be incorporated by reference.

1. An isolated aglycosylated plasminogen (PLG) polypeptide comprising an amino acid sequence having at least 95% sequence identity to the sequence set forth in SEQ ID NO:4, wherein the asparagine (Asn) residue at the position corresponding to position 289 of SEQ ID NO:4 has been substituted with an amino acid residue other than Asn, and wherein said PLG polypeptide retains an O-linked glycosylation site at the residue position corresponding to threonine-346 (Thr-346) of SEQ ID NO:4, wherein said PLG polypeptide is capable of being activated to a polypeptide capable of binding fibrin and having serine protease activity.

2. The isolated aglycosylated PLG polypeptide of claim 1, wherein the amino acid residue at the position corresponding to position 346 of SEQ ID NO:4 is threonine (Thr).

3. The isolated aglycosylated PLG polypeptide of claim 1, wherein the amino acid residue at the position corresponding to position 289 of SEQ ID NO:4 is selected from the group consisting of glutamine (Gln), histidine (His), lysine (Lys), Arginine (Arg), and aspartic acid (Asp).

4. The isolated aglycosylated PLG polypeptide of claim 3, wherein said PLG comprises the amino acid sequence set forth in SEQ ID NO:4, wherein the amino acid at position 289 of SEQ ID NO:4 is substituted with an amino acid residue selected from the group consisting of Gln, His, Lys, Arg, and Asp.

5. The isolated aglycosylated PLG polypeptide of claim 4, wherein said PLG comprises the amino acid sequence set forth in SEQ ID NO:6.

6. Aglycosylated plasmin obtained from the aglycosylated PLG polypeptide of claim 1.

7. A pharmaceutical composition comprising the aglycosylated plasmin of claim 6.

8. An isolated nucleic acid molecule comprising:
a) a nucleotide sequence encoding a plasminogen (PLG) polypeptide having at least 95% amino acid sequence identity to the sequence set forth in SEQ ID NO:4, wherein said PLG polypeptide comprises an amino acid residue other than asparagine (Asn) at the residue position corresponding to Asn-289 of SEQ ID NO:4, and wherein said PLG polypeptide retains an O-linked glycosylation site at the residue position corresponding to threonine-346 (Thr-346) of SEQ ID NO:4; or
b) a codon-optimized nucleotide sequence encoding a plasminogen (PLG) polypeptide having at least 95% amino acid sequence identity to the sequence set forth in SEQ ID NO:4, wherein said PLG polypeptide comprises an amino acid residue other than asparagine (Asn) at the residue position corresponding to Asn-289 of SEQ ID NO:4, wherein said codon-optimized nucleotide sequence is codon-optimized for expression in a plant of interest;
wherein said PLG polypeptide is capable of being activated to a polypeptide having serine protease activity.

9. The isolated nucleic acid molecule of claim 8, wherein said PLG polypeptide comprises a Thr residue at the residue position corresponding to Thr-346 of SEQ ID NO:4.

10. The isolated nucleic acid molecule of claim 8, wherein the amino acid residue at the residue position corresponding to Asn-289 of SEQ ID NO:4 is selected from the group consisting of glutamine (Gln), histidine (His), lysine (Lys), Arginine (Arg), and aspartic acid (Asp).

11. The isolated nucleic acid molecule of claim 10, wherein said PLG polypeptide comprises the amino acid sequence set forth in SEQ ID NO:4 with a Gln, His, Lys, Arg, or Asp residue substituted for Asn-289 of SEQ ID NO:4.

12. The isolated nucleic acid molecule of claim 11, wherein said PLG polypeptide comprises the amino acid sequence set forth in SEQ ID NO:6.

13. The isolated nucleic acid molecule of part a) of claim 8, wherein said nucleotide sequence encoding said PLG polypeptide is codon-optimized for expression in a plant of interest.

14. The isolated nucleic acid molecule of claim 8, wherein said nucleotide sequence encoding said PLG polypeptide comprises at least 70% plant-preferred codons.

15. The isolated nucleic acid molecule of claim 14, wherein said nucleotide sequence encoding said PLG polypeptide comprises the sequence set forth in SEQ ID NO:5.

16-22. (canceled)

23. An expression construct comprising the nucleic acid molecule of claim 8.

24. The expression construct of claim 23, wherein said nucleotide sequence encoding said PLG polypeptide is operably linked to:
a) a nucleotide sequence encoding a signal peptide that directs secretion of said PLG polypeptide;
b) a nucleotide sequence comprising a plant intron that is inserted upstream of said nucleotide sequence encoding said PLG polypeptide; and/or
c) a nucleotide sequence comprising a translation leader sequence.

25-31. (canceled)

32. A transformed plant or plant cell or plant nodule comprising the expression construct of claim 23.

33-35. (canceled)

36. A method for producing an aglycosylated plasminogen (PLG) polypeptide in a plant, the method comprising the steps of:
a) culturing in a culture medium under conditions suitable for expression of said PLG polypeptide a plant, plant cell, or plant nodule comprising an expression construct of claim 23; and
b) collecting said PLG polypeptide from at least one of said culture medium, plant, plant cell, or plant nodule.

37. (canceled)

38. The method of claim 36, wherein said plant, plant cell, or plant nodule is from a genus selected from the group consisting of the genus Spirodela, genus Wolffia, genus Wolfiella, genus Landoltia and genus Lemna.

39. (canceled)
